SEQUENCE LISTING (57 sequences)

SEQ ID NO: 1 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - protein, 210 aa, Mus musculus
MEWMRSRVGTLGLWVRLLLAVFLLGVYQAYPIPDSSPLLQFGGQVRQRYLYTDDDQDTEAHLEIREDGTVVGAAHRSPESLLELKALKPGVIQILGVKASRFLCQQPDGALYGSPHFDPEACSFRELLLEDGYNVYQSEAHGLPLRLPQKDSPNQDATSWGPVRFLPMPGLLHEPQDQAGFLPPEPPDVGSSDPLSMVEPLQGRSPSYAS

SEQ ID NO: 3 - protein, 209 aa, Canis lupus
  source 1..209: note = Canis lupus familiaris
MGWAEAGFEHLGLWVPVLAVLLLEACRAHPIPDSSPLLQFGGQVRQRYLYTDDAQETEAHLEIRADGTVVGAARQSPESLLELKALKPGVIQILGVKTSRFLCQGPDGTLYGSLHFDPVACSFRELLLEDGYNIYHSETLGLPLRLRPHNSAYRDLAPRGPARFLPLPGLLPAPPEPPGILAPEPPDVGSSDPLSMVGPSQGRSPSYAS

SEQ ID NO: 4 - DNA, 630 bp, Homo sapiens
  source 1..630: mol_type = other DNA
atggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctga

SEQ ID NO: 5 - DNA, 630 bp, synthetic construct
  misc_feature 1..630: note = Codon optimized Homo sapiens FGF21 - variant 1
  source 1..630: mol_type = other DNA
atggattctgatgagacaggcttcgagcacagcggcctgtgggtttcagttctggctggactgctgctgggagcctgtcaggcacaccctattccagatagcagccctctgctgcagttcggcggacaagtgcggcagagatacctgtacaccgacgacgcccagcagacagaagcccacctggaaatcagagaggatggcacagttggcggagccgccgatcagtctcctgaatctctgctccagctgaaggccctgaagcctggcgtgatccagatcctgggcgtgaaaaccagccggttcctgtgccaaagacctgacggcgccctgtatggcagcctgcactttgatcctgaggcctgcagcttcagagagctgctgcttgaggacggctacaacgtgtaccagtctgaggcccatggcctgcctctgcatctgcctggaaacaagagccctcacagagatcccgctcctagaggccctgccagatttctgcctcttcctggattgcctcctgctctgccagagcctcctggaattctggctcctcagcctcctgatgtgggcagctctgatcctctgagcatggtcggacctagccagggcagatctcctagctacgcctcttga

SEQ ID NO: 6 - DNA, 630 bp, synthetic construct
  misc_feature 1..630: note = Codon optimized Homo sapiens FGF21 - variant 2
  source 1..630: mol_type = other DNA
atggacagcgatgaaaccgggttcgagcacagcggtctgtgggtgtccgtgctggccggactgctcctgggagcctgtcaggcgcaccccatccctgactcctcgccgctgctgcaattcggcggacaagtccgccagagatacctgtacaccgacgacgcccagcagaccgaagcccacctggaaattcgggaggacgggactgtgggaggcgctgcagatcagtcacccgagtccctcctccaactgaaggccttgaagcccggcgtgattcagatcctgggcgtgaaaacttcccgcttcctttgccaacggccggatggagctctgtacggatccctgcacttcgaccccgaagcctgctcattccgcgagctgctccttgaggacggctataacgtgtaccagtctgaggcccatggactccccctgcatctgcccggcaacaagtcccctcaccgggatcctgccccaagaggcccagctcggtttctgcctctgccgggactgcctccagcgttgcccgaaccccctggtatcctggccccgcaaccacctgacgtcggttcgtcggacccgctgagcatggtcggtccgagccagggaaggtccccgtcctacgcatcctga

SEQ ID NO: 7 - DNA, 630 bp, synthetic construct
  misc_feature 1..630: note = Codon optimized Homo sapiens FGF21 - variant 3
  source 1..630: mol_type = other DNA
atggattccgacgaaactggatttgaacattcagggctgtgggtctctgtgctggctggactgctgctgggggcttgtcaggctcaccccatccctgacagctcccctctgctgcagttcggaggacaggtgcggcagagatacctgtataccgacgatgcccagcagacagaggcacacctggagatcagggaggacggaaccgtgggaggagcagccgatcagtctcccgagagcctgctgcagctgaaggccctgaagcctggcgtgatccagatcctgggcgtgaagacatctcggtttctgtgccagcggcccgacggcgccctgtacggctccctgcacttcgatcccgaggcctgttcttttagggagctgctgctggaggacggctacaacgtgtatcagagcgaggcacacggcctgccactgcacctgcctggcaataagtcccctcaccgcgatccagcacccaggggcccagcacgcttcctgcctctgccaggcctgccccctgccctgccagagccacccggcatcctggccccccagcctccagatgtgggctccagcgatcctctgtcaatggtggggccaagtcaggggcggagtccttcatacgcatcataa

SEQ ID NO: 8 - DNA, 633 bp, Mus musculus
  source 1..633: mol_type = other DNA
atggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctga

SEQ ID NO: 9 - DNA, 633 bp, synthetic construct
  misc_feature 1..633: note = Codon optimized Mus musculus FGF21
  source 1..633: mol_type = other DNA
atggaatggatgagaagcagagtgggcaccctgggcctgtgggtgcgactgctgctggctgtgtttctgctgggcgtgtaccaggcctaccccatccctgactctagccccctgctgcagtttggcggacaagtgcggcagagatacctgtacaccgacgacgaccaggacaccgaggcccacctggaaatccgcgaggatggcacagtcgtgggcgctgctcacagaagccctgagagcctgctggaactgaaggccctgaagcccggcgtgatccagatcctgggcgtgaaggccagcagattcctgtgccagcagcctgacggcgccctgtacggctctcctcacttcgatcctgaggcctgcagcttcagagagctgctgctggaggacggctacaacgtgtaccagtctgaggcccacggcctgcccctgagactgcctcagaaggacagccctaaccaggacgccacaagctggggacctgtgcggttcctgcctatgcctggactgctgcacgagccccaggatcaggctggctttctgcctcctgagcctccagacgtgggcagcagcgaccctctgagcatggtggaacctctgcagggcagaagccccagctacgcctcttga

SEQ ID NO: 10 - DNA, 629 bp, Canis lupus
  source 1..629: mol_type = other DNA; note = Canis lupus familiaris
atgggctgggccgaggccgggttcgagcacctgggactgtgggtccctgtgctggctgtgcttttgctggaagcctgccgggcacatccgatccctgactccagccccctcctacaatttggaggtcaagttcgacagcggtacctctacaccgacgatgcccaggagacagaggcccacctagagatcagggccgatggcacagggtgggggctgcccgccagagccctgaaagtctcctggagctgaaagccctaaagccaggggtcattcaaatcttgggagtcaaaacatccaggttcctgtgccagggcccagatgggacactatatggctcgctccatttcgaccctgtggcctgcagtttccgagaactgcttcttgaggatgggtacaacatctaccactccgagacccttggtctcccgcttcgcctgcgcccccacaactccgcataccgggacttggcaccccgcgggcctgcccgcttcctgccactgccaggcctgcttccagcacccccagagcctccagggatcctggccccggagcctcctgacgtgggctcctcggaccctctgagcatggtggggccttcacagggccggagtcccagctatgcttcctaa

SEQ ID NO: 11 - DNA, 630 bp, synthetic construct
  misc_feature 1..630: note = Codon optimized Canis lupus familiaris FGF21
  source 1..630: mol_type = other DNA
atgggatgggctgaggctggattcgaacacctgggactctgggtgcccgtcctggccgtgctgctcctggaggcttgcagggctcatcccatccctgacagctccccactcctgcagtttggaggacaggtgaggcagcggtacctgtataccgacgatgcccaggagacagaagctcacctggaaattcgggctgatggaacagtggtcggagctgcccgacagtccccagagtctctcctggaactgaaggccctcaaacccggagtgatccagattctgggcgtcaagacttctagattcctgtgccagggaccagacggcaccctgtacggcagcctgcatttcgatcctgtggcctgttcctttcgagagctcctgctcgaagacggctacaacatctatcactctgagaccctgggactcccactgcgactcagacctcataatagtgcctatcgagatctggctcccaggggcccagctaggtttctgccactccccggactgctccctgctccacctgagccacccggcattctggctccagaacctccagacgtgggctctagtgatccactgagtatggtcggcccctcacaggggaggtcacctagctacgccagctga

SEQ ID NO: 12 - DNA, 22 bp, synthetic construct
  misc_feature 1..22: note = Nucleotide sequence encoding miRT122a
  source 1..22: mol_type = other DNA
caaacaccattgtcacactcca

SEQ ID NO: 13 - DNA, 22 bp, synthetic construct
  misc_feature 1..22: note = Nucleotide sequence encoding miRT1
  source 1..22: mol_type = other DNA
ttacatacttctttacattcca

SEQ ID NO: 14 - DNA, 21 bp, synthetic construct
  misc_feature 1..21: note = Nucleotide sequence encoding miRT152
  source 1..21: mol_type = other DNA
ccaagttctgtcatgcactga

SEQ ID NO: 15 - DNA, 23 bp, synthetic construct
  misc_feature 1..23: note = Nucleotide sequence encoding miRT199a-5p
  source 1..23: mol_type = other DNA
gaacaggtagtctgaacactggg

SEQ ID NO: 16 - DNA, 22 bp, synthetic construct
  misc_feature 1..22: note = Nucleotide sequence encoding miRT199a-3p
  source 1..22: mol_type = other DNA
taaccaatgtgcagactactgt

SEQ ID NO: 17 - DNA, 21 bp, synthetic construct
  misc_feature 1..21: note = Nucleotide sequence encoding miRT215
  source 1..21: mol_type = other DNA
gtctgtcaattcataggtcat

SEQ ID NO: 18 - DNA, 21 bp, synthetic construct
  misc_feature 1..21: note = Nucleotide sequence encoding miRT192
  source 1..21: mol_type = other DNA
ggctgtcaattcataggtcag

SEQ ID NO: 19 - DNA, 22 bp, synthetic construct
  misc_feature 1..22: note = Nucleotide sequence encoding miRT148a
  source 1..22: mol_type = other DNA
acaaagttctgtagtgcactga

SEQ ID NO: 20 - DNA, 22 bp, synthetic construct
  misc_feature 1..22: note = Nucleotide sequence encoding miRT194
  source 1..22: mol_type = other DNA
tccacatggagttgctgttaca

SEQ ID NO: 21 - DNA, 20 bp, synthetic construct
  misc_feature 1..20: note = Nucleotide sequence encoding miRT124
  source 1..20: mol_type = other DNA
ggcattcaccgcgtgcctta

SEQ ID NO: 22 - DNA, 22 bp, synthetic construct
  misc_feature 1..22: note = Nucleotide sequence encoding miRT216
  source 1..22: mol_type = other DNA
tcacagttgccagctgagatta

SEQ ID NO: 23 - DNA, 24 bp, synthetic construct
  misc_feature 1..24: note = Nucleotide sequence encoding miRT125
  source 1..24: mol_type = other DNA
tcacaggttaaagggtctcaggga

SEQ ID NO: 24 - DNA, 22 bp, synthetic construct
  misc_feature 1..22: note = Nucleotide sequence encoding miRT133a
  source 1..22: mol_type = other DNA
cagctggttgaaggggaccaaa

SEQ ID NO: 25 - DNA, 22 bp, synthetic construct
  misc_feature 1..22: note = Nucleotide sequence encoding miRT206
  source 1..22: mol_type = other DNA
ccacacacttccttacattcca

SEQ ID NO: 26 - DNA, 22 bp, synthetic construct
  misc_feature 1..22: note = Nucleotide sequence encoding miRT130
  source 1..22: mol_type = other DNA
atgcccttttaacattgcactg

SEQ ID NO: 27 - DNA, 22 bp, synthetic construct
  misc_feature 1..22: note = Nucleotide sequence encoding miRT99
  source 1..22: mol_type = other DNA
cacaagatcggatctacgggtt

SEQ ID NO: 28 - DNA, 22 bp, synthetic construct
  misc_feature 1..22: note = Nucleotide sequence encoding miRT208a-5p
  source 1..22: mol_type = other DNA
gtataacccgggccaaaagctc

SEQ ID NO: 29 - DNA, 22 bp, synthetic construct
  misc_feature 1..22: note = Nucleotide sequence encoding miRT208a-3p
  source 1..22: mol_type = other DNA
acaagctttttgctcgtcttat

SEQ ID NO: 30 - DNA, 21 bp, synthetic construct
  misc_feature 1..21: note = Nucleotide sequence encoding miRT499-5p
  source 1..21: mol_type = other DNA
aaacatcactgcaagtcttaa

SEQ ID NO: 31 - DNA, 6343 bp, synthetic construct
  misc_feature 1..6343: note = pAAV-CAG-null
  source 1..6343: mol_type = other DNA
aattcgagctcggtacccgggaatcaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctactcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattggcctcgagcgcaagcttgagctagctcgatatcggcctaggctggatccgcgcggccgcaag

SEQ ID NO: 32 - DNA, 7319 bp, synthetic construct
  misc_feature 1..7319: note = pAAV-CAG-moFGF21-dmiRT
  source 1..7319: mol_type = other DNA
agtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttccactgaggcccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctactcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattggcctcgagcgcaagcttgagctagcgccaccatggaatggatgagaagcagagtgggcaccctgggcctgtgggtgcgactgctgctggctgtgtttctgctgggcgtgtaccaggcctaccccatccctgactctagccccctgctgcagtttggcggacaagtgcggcagagatacctgtacaccgacgacgaccaggacaccgaggcccacctggaaatccgcgaggatggcacagtcgtgggcgctgctcacagaagccctgagagcctgctggaactgaaggccctgaagcccggcgtgatccagatcctgggcgtgaaggccagcagattcctgtgccagcagcctgacggcgccctgtacggctctcctcacttcgatcctgaggcctgcagcttcagagagctgctgctggaggacggctacaacgtgtaccagtctgaggcccacggcctgcccctgagactgcctcagaaggacagccctaaccaggacgccacaagctggggacctgtgcggttcctgcctatgcctggactgctgcacgagccccaggatcaggctggctttctgcctcctgagcctccagacgtgggcagcagcgaccctctgagcatggtggaacctctgcagggcagaagccccagctacgcctcttgagaatgcgggcccggtacccccgacgcggccgctaattctagatcgcgaacaaacaccattgtcacactccagtatacacaaacaccattgtcacactccagatatcacaaacaccattgtcacactccaaggcgaacaaacaccattgtcacactccaaggctattctagatcgcgaattacatacttctttacattccagtatacattacatacttctttacattccagatatcattacatacttctttacattccaaggcgaattacatacttctttacattccaaggctacctgaggcccgggggtacctcttaattaactggcctcatgggccttccgctcactgcccgctttccagtcgggaaacctgtcgtgccagtcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaa

SEQ ID NO: 33 - DNA, 5678 bp, synthetic construct
  misc_feature 1..5678: note = pGG2-hAAT-null
  source 1..5678: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgacagatctgatatcatcgatgaattcgagctcggtacccggccgcagatttaggtgacactatagaatatgcatcactagtaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagctttgctctagactggaattcgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtccccctgatctgcggccgtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacctcgagacgcgtgatatcggatcccggccggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 34 - DNA, 6363 bp, synthetic construct
  misc_feature 1..6363: note = pGG2-hAAT-moFGF21
  source 1..6363: mol_type = other DNA
cgcgtgatatcggatcccggccggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgacagatctgatatcatcgatgaattcgagctcggtacccggccgcagatttaggtgacactatagaatatgcatcactagtaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagctttgctctagactggaattcgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtccccctgatctgcggccgtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacctcgagcgcaagcttgagctagcgccaccatggaatggatgagaagcagagtgggcaccctgggcctgtgggtgcgactgctgctggctgtgtttctgctgggcgtgtaccaggcctaccccatccctgactctagccccctgctgcagtttggcggacaagtgcggcagagatacctgtacaccgacgacgaccaggacaccgaggcccacctggaaatccgcgaggatggcacagtcgtgggcgctgctcacagaagccctgagagcctgctggaactgaaggccctgaagcccggcgtgatccagatcctgggcgtgaaggccagcagattcctgtgccagcagcctgacggcgccctgtacggctctcctcacttcgatcctgaggcctgcagcttcagagagctgctgctggaggacggctacaacgtgtaccagtctgaggcccacggcctgcccctgagactgcctcagaaggacagccctaaccaggacgccacaagctggggacctgtgcggttcctgcctatgcctggactgctgcacgagccccaggatcaggctggctttctgcctcctgagcctccagacgtgggcagcagcgaccctctgagcatggtggaacctctgcagggcagaagccccagctacgcctcttgagaatgcgggcccggtacccccgacgcggcc

SEQ ID NO: 35 - DNA, 4403 bp, synthetic construct
  misc_feature 1..4403: note = pAAV-CMV-null
  source 1..4403: mol_type = other DNA
cgacggtaccagcgctgtcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctacctagacatggctcgacagatcagcgctcatgctctggaagatctcgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatctgtagttaatgattaacccgccatgctacttatctacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactaggctagctattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactatagaatacgactcactatagggagacgctagcgtcga

SEQ ID NO: 36 - DNA, 5073 bp, synthetic construct
  misc_feature 1..5073: note = pAAV-CMV-moFGF21
  source 1..5073: mol_type = other DNA
ggggctagcgccaccatggaatggatgagaagcagagtgggcaccctgggcctgtgggtgcgactgctgctggctgtgtttctgctgggcgtgtaccaggcctaccccatccctgactctagccccctgctgcagtttggcggacaagtgcggcagagatacctgtacaccgacgacgaccaggacaccgaggcccacctggaaatccgcgaggatggcacagtcgtgggcgctgctcacagaagccctgagagcctgctggaactgaaggccctgaagcccggcgtgatccagatcctgggcgtgaaggccagcagattcctgtgccagcagcctgacggcgccctgtacggctctcctcacttcgatcctgaggcctgcagcttcagagagctgctgctggaggacggctacaacgtgtaccagtctgaggcccacggcctgcccctgagactgcctcagaaggacagccctaaccaggacgccacaagctggggacctgtgcggttcctgcctatgcctggactgctgcacgagccccaggatcaggctggctttctgcctcctgagcctccagacgtgggcagcagcgaccctctgagcatggtggaacctctgcagggcagaagccccagctacgcctcttgagaatgcgggcccggtaccccctcgacggtaccagcgctgtcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctacctagacatggctcgacagatcagcgctcatgctctggaagatctcgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatctgtagttaatgattaacccgccatgctacttatctacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactaggctagctattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactatagaatacgactcactatagggagacgctagcgtcga

SEQ ID NO: 37 - DNA, 7292 bp, synthetic construct
  misc_feature 1..7292: note = pAAV-CAG-cloFGF21-dmiRT
  source 1..7292: mol_type = other DNA
ggccgctaattctagatcgcgaacaaacaccattgtcacactccagtatacacaaacaccattgtcacactccagatatcacaaacaccattgtcacactccaaggcgaacaaacaccattgtcacactccaaggctattctagatcgcgaattacatacttctttacattccagtatacattacatacttctttacattccagatatcattacatacttctttacattccaaggcgaattacatacttctttacattccaaggctacctgaggcccgggggtacctcttaattaactggcctcatgggccttccgctcactgcccgctttccagtcgggaaacctgtcgtgccagtcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaaagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttccactgaggcccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctactcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattggcctcgagcgcaagcttgagctagcgccaccatgggatgggctgaggctggattcgaacacctgggactctgggtgcccgtcctggccgtgctgctcctggaggcttgcagggctcatcccatccctgacagctccccactcctgcagtttggaggacaggtgaggcagcggtacctgtataccgacgatgcccaggagacagaagctcacctggaaattcgggctgatggaacagtggtcggagctgcccgacagtccccagagtctctcctggaactgaaggccctcaaacccggagtgatccagattctgggcgtcaagacttctagattcctgtgccagggaccagacggcaccctgtacggcagcctgcatttcgatcctgtggcctgttcctttcgagagctcctgctcgaagacggctacaacatctatcactctgagaccctgggactcccactgcgactcagacctcataatagtgcctatcgagatctggctcccaggggcccagctaggtttctgccactccccggactgctccctgctccacctgagccacccggcattctggctccagaacctccagacgtgggctctagtgatccactgagtatggtcggcccctcacaggggaggtcacctagctacgccagctgagc

SEQ ID NO: 38 - DNA, 6278 bp, synthetic construct
  misc_feature 1..6278: note = pGG2-hAAT-cloFGF21
  source 1..6278: mol_type = other DNA
ggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgacagatctgatatcatcgatgaattcgagctcggtacccggccgcagatttaggtgacactatagaatatgcatcactagtaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagctttgctctagactggaattcgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtccccctgatctgcggccgtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatgggatgggctgaggctggattcgaacacctgggactctgggtgcccgtcctggccgtgctgctcctggaggcttgcagggctcatcccatccctgacagctccccactcctgcagtttggaggacaggtgaggcagcggtacctgtataccgacgatgcccaggagacagaagctcacctggaaattcgggctgatggaacagtggtcggagctgcccgacagtccccagagtctctcctggaactgaaggccctcaaacccggagtgatccagattctgggcgtcaagacttctagattcctgtgccagggaccagacggcaccctgtacggcagcctgcatttcgatcctgtggcctgttcctttcgagagctcctgctcgaagacggctacaacatctatcactctgagaccctgggactcccactgcgactcagacctcataatagtgcctatcgagatctggctcccaggggcccagctaggtttctgccactccccggactgctccctgctccacctgagccacccggcattctggctccagaacctccagacgtgggctctagtgatccactgagtatggtcggcccctcacaggggaggtcacctagctacgccagctgagc

SEQ ID NO: 39 - DNA, 6278 bp, synthetic construct
  misc_feature 1..6278: note = pGG2-hAAT-hsFGF21
  source 1..6278: mol_type = other DNA
gatctgatatcatcgatgaattcgagctcggtacccggccgcagatttaggtgacactatagaatatgcatcactagtaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagctttgctctagactggaattcgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtccccctgatctgcggccgtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctgagcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgaca

SEQ ID NO: 40 - DNA, 6278 bp, synthetic construct
  misc_feature 1..6278: note = pGG2-hAAT-hsoFGF21 variant 1
  source 1..6278: mol_type = other DNA
gatctgatatcatcgatgaattcgagctcggtacccggccgcagatttaggtgacactatagaatatgcatcactagtaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagctttgctctagactggaattcgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtccccctgatctgcggccgtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatggattctgatgagacaggcttcgagcacagcggcctgtgggtttcagttctggctggactgctgctgggagcctgtcaggcacaccctattccagatagcagccctctgctgcagttcggcggacaagtgcggcagagatacctgtacaccgacgacgcccagcagacagaagcccacctggaaatcagagaggatggcacagttggcggagccgccgatcagtctcctgaatctctgctccagctgaaggccctgaagcctggcgtgatccagatcctgggcgtgaaaaccagccggttcctgtgccaaagacctgacggcgccctgtatggcagcctgcactttgatcctgaggcctgcagcttcagagagctgctgcttgaggacggctacaacgtgtaccagtctgaggcccatggcctgcctctgcatctgcctggaaacaagagccctcacagagatcccgctcctagaggccctgccagatttctgcctcttcctggattgcctcctgctctgccagagcctcctggaattctggctcctcagcctcctgatgtgggcagctctgatcctctgagcatggtcggacctagccagggcagatctcctagctacgcctcttgagcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgaca

SEQ ID NO: 41 - DNA, 6278 bp, synthetic construct
  misc_feature 1..6278: note = pGG2-hAAT-hsoFGF21 variant 2
  source 1..6278: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgacagatctgatatcatcgatgaattcgagctcggtacccggccgcagatttaggtgacactatagaatatgcatcactagtaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagctttgctctagactggaattcgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtccccctgatctgcggccgtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatggacagcgatgaaaccgggttcgagcacagcggtctgtgggtgtccgtgctggccggactgctcctgggagcctgtcaggcgcaccccatccctgactcctcgccgctgctgcaattcggcggacaagtccgccagagatacctgtacaccgacgacgcccagcagaccgaagcccacctggaaattcgggaggacgggactgtgggaggcgctgcagatcagtcacccgagtccctcctccaactgaaggccttgaagcccggcgtgattcagatcctgggcgtgaaaacttcccgcttcctttgccaacggccggatggagctctgtacggatccctgcacttcgaccccgaagcctgctcattccgcgagctgctccttgaggacggctataacgtgtaccagtctgaggcccatggactccccctgcatctgcccggcaacaagtcccctcaccgggatcctgccccaagaggcccagctcggtttctgcctctgccgggactgcctccagcgttgcccgaaccccctggtatcctggccccgcaaccacctgacgtcggttcgtcggacccgctgagcatggtcggtccgagccagggaaggtccccgtcctacgcatcctgagcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 42 - DNA, 6278 bp, synthetic construct
  misc_feature 1..6278: note = pGG2-hAAT-hsoFGF21 variant 3
  source 1..6278: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgacagatctgatatcatcgatgaattcgagctcggtacccggccgcagatttaggtgacactatagaatatgcatcactagtaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagcttgcgaattccagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagctttgctctagactggaattcgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtccccctgatctgcggccgtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatggattccgacgaaactggatttgaacattcagggctgtgggtctctgtgctggctggactgctgctgggggcttgtcaggctcaccccatccctgacagctcccctctgctgcagttcggaggacaggtgcggcagagatacctgtataccgacgatgcccagcagacagaggcacacctggagatcagggaggacggaaccgtgggaggagcagccgatcagtctcccgagagcctgctgcagctgaaggccctgaagcctggcgtgatccagatcctgggcgtgaagacatctcggtttctgtgccagcggcccgacggcgccctgtacggctccctgcacttcgatcccgaggcctgttcttttagggagctgctgctggaggacggctacaacgtgtatcagagcgaggcacacggcctgccactgcacctgcctggcaataagtcccctcaccgcgatccagcacccaggggcccagcacgcttcctgcctctgccaggcctgccccctgccctgccagagccacccggcatcctggccccccagcctccagatgtgggctccagcgatcctctgtcaatggtggggccaagtcaggggcggagtccttcatacgcatcataagcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 43 - DNA, 133 bp, synthetic construct
  misc_feature 1..133: note = Chimeric intron composed of introns from human beta-globin and immunoglobulin heavy chain genes
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 44 - DNA, 1671 bp, synthetic construct
  misc_feature 1..1671: note = CAG promoter
  source 1..1671: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 45 - DNA, 212 bp, synthetic construct
  misc_feature 1..212: note = CMV promoter
  source 1..212: mol_type = other DNA
gtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagct

SEQ ID NO: 46 - DNA, 380 bp, synthetic construct
  misc_feature 1..380: note = CMV enhancer
  source 1..380: mol_type = other DNA
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 47 - DNA, 397 bp, synthetic construct
  misc_feature 1..397: note = hAAT promoter
  source 1..397: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 48 - DNA, 128 bp, synthetic construct
  misc_feature 1..128: note = Truncated AAV2 5'ITR
  source 1..128: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 49 - DNA, 128 bp, synthetic construct
  misc_feature 1..128: note = Truncated AAV2 3'ITR
  source 1..128: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 50 - DNA, 122 bp, synthetic construct
  misc_feature 1..122: note = SV40 polyadenylation signal
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 51 - DNA, 449 bp, synthetic construct
  misc_feature 1..449: note = Rabbit beta-globin polyadenylation signal
  source 1..449: mol_type = other DNA
gatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 52 - DNA, 592 bp, synthetic construct
  misc_feature 1..592: note = CMV promoter and CMV enhancer
  source 1..592: mol_type = other DNA
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagct

SEQ ID NO: 53 - DNA, 154 bp, synthetic construct
  misc_feature 1..154: note = Hepatocyte control region (HCR) enhancer from apolipoprotein E
  source 1..154: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagc

SEQ ID NO: 54 - DNA, 651 bp, synthetic construct
  misc_feature 1..651: note = mini/aP2 promoter
  source 1..651: mol_type = other DNA
gattaacccgccatgctacttatctactcgacattgattattgactaggggaattccagcaggaatcaggtagctggagaatcgcacagagccatgcgattcttggcaagccatgcgacaaaggcagaaatgcacatttcacccagagagaagggattgatgtcagcaggaagtcaccacccagagagcaaatggagttcccagatgcctgacatttgccttcttactggatcagagttcactagtggaagtgtcacagcccaaacactcccccaaagctcagcccttccttgccttgtaacaatcaagccgctcctggatgaactgctccgccctctgtctctttggcagggttggagcccactgtggcctgagcgacttctatggctcccttttctgtgattttcatggtttctgagctcttttcccccgctttatgattttctctttttgtctctctcttgctaaacctccttcgtatatatgccctctcaggtttcatttctgaatcatctactgtgaactattcccattgtttgccagaagccccctggttcttccttctagacaccaggcaaggggcaggaggtaagaggcaggagtccataaaacagccctgagagcctgctgggtcagtgcctgctgtcagaa

SEQ ID NO: 55 - DNA, 722 bp, synthetic construct
  misc_feature 1..722: note = mini/UCP1 promoter
  source 1..722: mol_type = other DNA
gacgtcacagtgggtcagtcacccttgatcacactgcaccagtcttcacctttccacgcttcctgccagagcatgaatcaggctctctggggataccggcctcacccctactgaggcaaactttctcccacttctcagaggctctgagggcagcaaggtcagccctttctttggaatctagaaccactccctgtcttgagctgacatcacagggcaggcagatgcagcagggaagggcctgggactgggacgttcatcctacaagaaagctgtggaacttttcagcaacatctcagaaatcagatcgcacttattcaaaggagccaggccctgctctgcgccctggtggaggctcctcatgtgaagagtgacaaaaggcaccatgttgtggatacggggcgaagcccctccggtgtgtcctccaggcatcatcaggaactagtgccaaagcagaggtgctggccagggctttgggagtgacgcgcgtctgggaggcttgtgcgcccagggcacgcccctgccgattcccactagcaggtcttgggggacctgggccggctctgcccctcctccagcaatcgggctataaagctcttccaagtcagggcgcagaagtgccgggcgatccgggcttaaagagcgagaggaagggacgctcacctttgagctcctccacaaatagccctggtggctgccacagaagttcgaagttgagagttcgg

SEQ ID NO: 56 - DNA, 326 bp, synthetic construct
  misc_feature 1..326: note = C5-12 promoter
  source 1..326: mol_type = other DNA
cggccgtccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgcca

SEQ ID NO: 57 - DNA, 4930 bp, synthetic construct
  misc_feature 1..4930: note = pAAV-EF1a-mmFGF21-pA
  source 1..4930: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgactgctagcacgcgtgatatcaatggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctgagatatcaaagaattctaagcttgtcgacgaatgcaattgttgttaattaattgttaacttgtttattgcagcttataatggttacaaataaagcaatagcatcacaaatttcacaaataaagcatttttttcactgcattctagttgtggtttgtccaaactcatcaatgtatcttagtcgagttaattaacggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2026/0139021 A1
(43) Pub. Date: May 21, 2026

(54) VIRAL EXPRESSION CONSTRUCT COMPRISING A FIBROBLAST GROWTH FACTOR (FGF21) CODING SEQUENCE

(71) Applicant: Universitat Autònoma de Barcelona, Cerdanyola del Valles (ES)

(72) Inventors: Fàtima BOSCH TUBERT, Cerdanyola del Valles (ES); Verónica JIMÉNEZ CENZANO, Cerdanyola del Valles (ES); Claudia JAMBRINA PALLARES, Cerdanyola del Valles (ES)

(21) Appl. No.: 19/418,207

(22) Filed: Dec. 12, 2025

Related U.S. Application Data
(62) Division of application No. 18/484,279, filed on Oct. 10, 2023, now Pat. No. 12,559,533.
Application No. 18/484,279 is a continuation of application No. 16/615,854, now abandoned.

(30) Foreign Application Priority Data
May 24, 2017 (EP) 17172818.1

(51) Int. Cl. C07K 14/50; A01K 67/0275; A61K 35/761; C12N 15/113; C12N 15/85; C12N 15/86

(52) U.S. Cl. CPC C07K 14/50; A01K 67/0275; A61K 35/761; C12N 15/113; C12N 15/86; A01K 2207/25; A01K 2227/105; A01K 2267/0362; C12N 2015/8518; C12N 2310/141; C12N 2750/14143; C12N 2799/02

(57) ABSTRACT

The invention relates to a viral expression construct and related viral vector and nucleic acid molecule and composition and to their use wherein said construct and vector are suitable for expression in a mammal and comprise a nucleotide sequence encoding a Fibroblast growth factor 21 (FGF21) to be expressed in liver, adipose tissue and/or skeletal muscle.

### REFERENCE TO SEQUENCE LISTING SUBMITTED ELECTRONICALLY

The content of the electronically submitted sequence listing (Name: 5344 0040004 Seqlisting_ST26.xml; Size: 161,788 bytes; Date of Creation: Dec. 9, 2025) filed with the application is incorporated herein by reference in its entirety.

### FIELD OF THE INVENTION

The invention pertains to the medical field, comprising gene therapy compositions for use in the treatment of a metabolic disorder, use in the treatment of liver inflammation and/or fibrosis, use in the treatment of cancer and/or use in extending healthy lifespan in mammals, particularly in human beings.

### BACKGROUND OF THE INVENTION

The prevalence of diabetes is growing at an alarming rate and is a major health problem worldwide. Obesity is strongly associated with insulin resistance and type 2 diabetes (T2D) (Moller, D. E., and Flier, J. S., 1991. N. Engl. J. Med. 325:938-948). Moreover, obesity increases the risk of mortality (Peeters, A. et al., 2003. Ann. Intern. Med. 138:24-32) and is also a significant risk factor for heart disease, immune dysfunction, hypertension, arthritis, neurodegenerative diseases, and certain types of cancer (Roberst, D. L. et al., 2010. Annu. Rev. Med. 61:301-316: Spiegelman. B. M. et al., 1993. J. Biol. Chem. 268:6823-6826: Whitmer, R. A., 2007. Curr. Alzheimer Res. 4:117-122). Despite the clinical significance of T2D and obesity, no effective treatments are available. Hence, there is an urgent need for novel and safe approaches to prevent and combat the current T2D-obesity epidemic. Recently, it has become widely accepted that obesity is an important risk factor for cancer (Roberst, D. L. et al., 2010. Annu. Rev. Med. 61:301-316). Given the current obesity epidemic, obesity-related cancer risks are a clinically important concern for which novel and safe approaches are urgently needed. An increase in weight and insulin resistance is also associated with aging. Hence, novel and safe approaches to prevent and reverse obesity and diabetes are needed to extend healthy lifespan. Liver fibrosis is an excessive accumulation of extracellular matrix proteins, e.g. collagen, resulting predominantly from chronic liver inflammation. Advanced liver fibrosis will lead to liver cirrhosis, portal hypertension and liver failure. Thus, novel and safe antifibrotic therapeutics are required. Fibroblast growth factor 21 (FGF21), a growth factor predominantly secreted by the liver, but also by adipose tissue and pancreas (Muise, E. S. et al., 2008. Mol. Pharmacol. 74:403-412), has been shown to increase brown adipose tissue (BAT) growth and expression of thermogenic genes in BAT and white adipose tissue (WAT), stimulating energy expenditure (Coskun, T. et al., 2008. Endocrinology 149:6018-6027; Fisher, F. M. et al., 2012. Genes Dev. 26:271-281; Kharitonenkov, A. et al., 2005. J. Clin. Invest 115:1627-1635; Konishi, M. et al., 2000. J. Biol. Chem. 275:12119-12122; Tomlinson, E. et al., 2002. Endocrinology 143:1741-1747; Xu, J. et al., 2009. Diabetes 58:250-259). Overexpression of FGF21 in transgenic mice protected them from diet-induced obesity (Kharitonenkov, A. et al., 2005. J. Clin. Invest 115:1627-1635) and the administration of FGF21 to ob/ob, db/db or high fat diet (HFD)-fed mice or to obese ZDF rats promoted a robust reduction in adiposity, significantly lowered blood glucose and triglycerides, decreased fasting insuling levels and improved insulin sensitivity (Coskun, T. et al., 2008. Endocrinology 149:6018-6027; Kharitonenkov, A. et al., 2005. J. Clin. Invest 115:1627-1635; Xu, J. et al., 2009. Diabetes 58:250-259; Adams, A. C. et al., 2012. PLOS. One. 7: e38438.; Berglund, E. D. et al., 2009. Endocrinology 150:4084-4093). Moreover, the administration of FGF21 to obese diabetic rhesus monkeys dramatically reduced fasting plasma glucose, fructosamine, triglyceride, insulin and glucagon levels and induced a small but significant weight loss (Kharitonenkov, A. et al., 2007. Endocrinology 148:774-781).

Native FGF21 protein exhibits poor pharmacokinetic characteristics. It has a short half-life, and it is susceptible to in vivo proteolytic degradation and in vitro aggregation (Huang, J. et al., 2013. J Pharmacol Exp Ther. 346 (2): 270-80; So, W. Y. and Leung, P. S. 2016. Med Res Rev. 36 (4): 672-704; Zhang, J. and Li, Y. 2015. Front Endocrinol (Lausanne). 6:168). Various engineering approaches have been developed to extend the half-life and to improve the stability and solubility of FGF21. Currently, two engineered FGF21 mimetics (LY2405319 and PF-05231023) are being tested in humans. Nevertheless, those FGF21 mimetics require multiple administrations, which poses a significant burden to the patients. Moreover, engineered FGF21 mimetics/analogs may exhibit a higher risk of immunogenicity than native FGF21, e.g. patients treated with LY2405319 developed injection site reactions, anti-drug antibodies and a serious hypersensitivity reaction (Gaich, G. et al., 2013. Cell Metab. 18 (3): 333-40).

Therefore there is still a need for new treatments for diabetes and/or obesity and/or liver inflammation and/or fibrosis and/or cancer and/or extending healthy lifespan which do not have all the drawbacks of existing treatments.

### DESCRIPTION OF THE INVENTION

The inventors designed improved gene therapy strategies based on adeno-associated viral (AAV) vector-mediated FGF21 gene transfer to liver, adipose tissue and/or skeletal muscle to counteract metabolic disorders, preferably diabetes and/or obesity. The gene therapy of the invention may also be used to counteract liver inflammation and/or fibrosis. Additionally, the gene therapy of the invention may also be used for the extension of healthy lifespan by counteracting metabolic disorders associated with aging, preferably diabetes and/or obesity. Additionally, the gene therapy of the invention may also be used to counteract cancer, preferably liver cancer.

Generation of single-vector gene constructs allows the in vivo production of native FGF21, which should result in reduced risk of immunogenicity or other toxicities. However, the skilled person knows that native FGF21 may be susceptible to in vivo proteolytic degradation and/or have a fast in vivo clearance rate. All vectors tested in the experimental part were found to be able to enable long-lasting secretion of stable native FGF21 into the bloodstream. Efficacy is maintained even with a single administration of gene transfer vectors.

Therefore the generation of such AAV vectors for in vivo production of native FGF21 is not routine for a person skilled in the art, as demonstrated in the experimental part.

### Viral Expression Construct

In a first aspect there is provided a viral expression construct suitable for expression in a mammal and comprising a nucleotide sequence encoding a Fibroblast growth factor 21 (FGF21) to be expressed in liver, adipose tissue and/or skeletal muscle.

The definition of “viral expression construct”, “suitable for expression in a mammal” “liver”, “adipose tissue” and “skeletal muscle” has been provided in the part of the description entitled “general definitions”.

A preferred nucleotide sequence encoding a FGF21 present in the viral expression construct of the invention has at least 60%, 65%, 70%, 75%, 80%, 85%, 90%, 95%, 97%, 98%, 99%, 100% identity with SEQ ID NO: 4, 5, 6, 7, 8, 9, 10 or 11. Identity may be assessed over the whole SEQ ID NO or over part thereof as explained in the part of the description entitled “general definitions”.

A more preferred nucleotide sequence encoding a human FGF21 present in the viral expression construct of the invention has at least 60%, 65%, 70%, 75%, 80%, 85%, 90%, 95%, 97%, 98%, 99%, 100% identity with SEQ ID NO: 4, 5, 6 or 7. Identity may be assessed over the whole SEQ ID NO or over part thereof as explained in the part of the description entitled “general definitions”. SEQ ID NO: 4 is a nucleotide sequence encoding human FGF21. SEQ ID NO: 5 is a codon optimized nucleotide sequence encoding human FGF21, variant 1. SEQ ID NO: 6 is a codon optimized nucleotide sequence encoding human FGF21, variant 2. SEQ ID NO: 7 is a codon optimized nucleotide sequence encoding human FGF21, variant 3. Variant 1, variant 2 and variant 3 encode for the same human FGF21 protein and were obtained by different algorithms of codon optimization. Another preferred nucleotide sequence encoding mouse FGF21 present in the viral expression construct of the invention has at least 60%, 65%, 70%, 75%, 80%, 85%, 90%, 95%, 97%, 98%, 99%, 100% identity with SEQ ID NO: 8 or 9. Identity may be assessed over the whole SEQ ID NO or over part thereof as explained in the part of the description entitled “general definitions”. SEQ ID NO: 8 is a nucleotide sequence encoding mouse FGF21. SEQ ID NO: 9 is a codon optimized nucleotide sequence encoding mouse FGF21. Another preferred nucleotide sequence encoding canine FGF21 present in the viral expression construct of the invention has at least 60%, 65%, 70%, 75%, 80%, 85%, 90%, 95%, 97%, 98%, 99%, 100% identity with SEQ ID NO: 10 or 11. Identity may be assessed over the whole SEQ ID NO or over part thereof as explained in the part of the description entitled “general definitions”. SEQ ID NO: 10 is a nucleotide sequence encoding canine FGF21. SEQ ID NO: 11 is a codon optimized nucleotide sequence encoding canine FGF21. The nucleotide sequence encoding a FGF21 may be derived from any FGF21 gene or FGF21 coding sequence, preferably from human, mouse or dog; or a mutated FGF21 gene or FGF21 coding sequence, or a codon optimized FGF21 gene or FGF21 coding sequence, preferably from human, mouse or dog.

A FGF21 as used herein exerts at least a detectable level of an activity of a FGF21 as known to the skilled person. An activity of a FGF21 is to increase insulin sensitivity. This activity could be assessed using the insulin tolerance test, as described in the experimental part, preferably as in example 8 or 9.

In an embodiment there is provided a viral expression construct as described above, wherein the nucleotide sequence encoding a FGF21 suitable for expression in a mammal is selected from the group consisting of:
(a) a nucleotide sequence encoding a polypeptide comprising an amino acid sequence that has at least 60% sequence identity with the amino acid sequence of SEQ ID NO: 1, 2 or 3.
(b) a nucleotide sequence that has at least 60% sequence identity with the nucleotide sequence of SEQ ID NO: 4, 5, 6, 7, 8, 9, 10 or 11.
(c) a nucleotide sequence the sequence of which differs from the sequence of a nucleotide sequence of (b) due to the degeneracy of the genetic code.

A preferred nucleotide sequence encoding a FGF21 suitable for expression in a mammal encodes a polypeptide comprising an amino acid sequence that has at least 60%, 65%, 70%, 75%, 80%, 85%, 90%, 95%, 97%, 98%, 99%, 100% identity with SEQ ID NO: 1, 2 or 3. Identity may be assessed over the whole SEQ ID NO or over part thereof as explained in the part of the description entitled “general definitions”. SEQ ID NO: 1 is an amino acid sequence of human FGF21. SEQ ID NO: 2 is an amino acid sequence of murine FGF21. SEQ ID NO: 3 is an amino acid sequence of canine FGF21.

In an embodiment there is provided a viral expression construct as described above comprising a nucleotide sequence encoding a FGF21 suitable for expression in a mammal and at least one of elements a), b), c), d) and e):
(a) a liver-specific promoter
(b) an adipose tissue-specific promoter
(c) a combination of an ubiquitous promoter and at least one nucleotide sequence encoding a target sequence of a microRNA expressed in the liver and at least one nucleotide sequence encoding a target sequence of a microRNA expressed in the heart, wherein said combination enables specific expression in adipose tissue
(d) a skeletal muscle promoter and
(e) a combination of an ubiquitous promoter and an adeno-associated virus (AAV) vector sequence, wherein said combination enables specific expression in skeletal muscle.

A “target sequence of a microRNA expressed in the liver” or “target sequence of a miRNA expressed in the liver” or “binding site of a microRNA expressed in the liver” refers to a nucleotide sequence which is complementary or partially complementary to at least a portion of a microRNA expressed in the liver. Similarly, a “target sequence of a microRNA expressed in the heart” or “target sequence of a miRNA expressed in the heart” or “binding site of a microRNA expressed in the heart” refers to a nucleotide sequence which is complementary or partially complementary to at least a portion of a microRNA expressed in the heart. A portion of a microRNA expressed in the liver or a portion of a microRNA expressed in the heart, as defined herein, means a nucleotide sequence of at least five or at least six consecutive nucleotides of said microRNA. The binding site sequence can have perfect complementarity to at least a portion of an expressed microRNA, meaning that the sequences can be a perfect match, no mismatch may occur. Alternatively, the binding site sequence can be partially complementary to at least a portion of an expressed microRNA, meaning that one mismatch/five, six consecutive nucleotides may occur. Partially complementary binding sites preferably contain perfect or near perfect complementarity to the seed region of the microRNA, meaning that no mismatch (perfect complementarity) or one mismatch/five, six consecutive nucleotides (near perfect complementarity) may occur between the seed region of the microRNA and its binding site. The seed region of the microRNA consists of the 5′ region of the microRNA from about nucleotide 2 to about nucleotide 8 of the microRNA (i.e. 6 nucleotides). The portion as defined herein is preferably the seed region of said microRNA. Degradation of the messenger RNA (mRNA) containing the target sequence for a microRNA expressed in the liver or a microRNA expressed in the heart may be through the RNA interference pathway or via direct translational control (inhibition) of the mRNA. The invention is not limited by the pathway ultimately utilized by the miRNA in inhibiting expression of the transgene or encoded protein. In the context of the invention, a nucleotide sequence encoding a target sequence of a microRNA expressed in the liver may be replaced by a nucleotide sequence comprising a nucleotide sequence that has at least 60% sequence identity or similarity with SEQ ID NO: 12 or 14-23. A more preferred nucleotide sequence has at least 60%, 65%, 70%, 75%, 80%, 85%, 90%, 95%, 97%, 98%, 99%, 100% identity with SEQ ID NO: 12 or 14-23. Identity may be assessed over the whole SEQ ID NO or over part thereof as explained in the part of the description entitled “general definitions”. In one embodiment, the nucleotide sequence encoding a target sequence of a microRNA expressed in the liver may be replaced by a nucleotide sequence comprising a nucleotide sequence that has at least 60% sequence identity or similarity with SEQ ID NO: 12. A more preferred nucleotide sequence has at least 60%, 65%, 70%, 75%, 80%, 85%, 90%, 95%, 97%, 98%, 99%, 100% identity with SEQ ID NO: 12. In a further embodiment, at least one copy of a nucleotide sequence encoding a target sequence of a microRNA expressed in the liver, as defined in SEQ ID NO: 12 or 14-23, is present in the viral expression construct of the invention. In a further embodiment, two, three, four, five, six, seven or eight copies of a nucleotide sequence encoding a target sequence of a liver-specific microRNA, as defined in SEQ ID NO: 12 or 14-23, are present in the viral expression construct of the invention. In a preferred embodiment one, two, three, four, five, six, seven or eight copies of a nucleotide sequence encoding miRT122a (SEQ ID NO: 12) are present in the viral expression construct of the invention.

A target sequence of a microRNA expressed in the liver as used herein exerts at least a detectable level of activity of a target sequence of a microRNA expressed in the liver as known to the skilled person. An activity of a target sequence of a microRNA expressed in the liver is to bind to its cognate, microRNA expressed in the liver and, when operatively linked to a transgene, to mediate detargeting of transgene expression in the liver. This activity may be assessed by measuring the levels of transgene expression in the liver by qPCR, as described in the experimental part.

In the context of the invention, a nucleotide sequence encoding a target sequence of a microRNA expressed in the heart may be replaced by a nucleotide sequence comprising a nucleotide sequence that has a least 60% sequence identity or similarity with SEQ ID NO: 13 or 23-30. A preferred nucleotide sequence has at least 60%, 65%, 70%, 75%, 80%, 85%, 90%, 95%, 97%, 98%, 99%, 100% identity with SEQ ID NO: 13 or 23-30. Identity may be assessed over the whole SEQ ID NO or over part thereof as explained in the part of the description entitled “general definitions”. In one embodiment, the nucleotide sequence encoding a target sequence of a microRNA expressed in the heart may be replaced by a nucleotide sequence comprising a nucleotide sequence that has at least 60% sequence identity or similarity with SEQ ID NO: 13. A preferred nucleotide sequence has at least 60%, 65%, 70%, 75%, 80%, 85%, 90%, 95%, 97%, 98%, 99%, 100% identity with SEQ ID NO: 13. In a further embodiment, at least one copy of a nucleotide sequence encoding a target sequence of a microRNA expressed in the heart, as defined in SEQ ID NO: 13 or 23-30, is present in the viral expression construct of the invention. In a further embodiment, two, three, four, five, six, seven or eight copies of a nucleotide sequence encoding a target sequence of a heart-specific microRNA, as defined in SEQ ID NO: 13 or 23-30, are present in the viral expression construct of the invention. In a preferred embodiment one, two, three, four, five, six, seven or eight copies of a nucleotide sequence encoding miRT1 (SEQ ID NO: 13), are present in the viral expression construct of the invention.

An activity of a target sequence of a microRNA expressed in the heart is to bind to its cognate, microRNA expressed in the heart and, when operatively linked to a transgene, to mediate detargeting of transgene expression in the heart. This activity may be assessed by measuring the levels of transgene expression in the heart by qPCR, as described in the experimental part.

In an embodiment there is provided a viral expression construct as described above, wherein the nucleotide sequence encoding a target sequence of a microRNA expressed in the liver and the nucleotide sequence encoding a target sequence of a microRNA expressed in the heart is selected from a group consisting of sequences SEQ ID NO: 12 to 30 and/or combinations thereof.

In one embodiment, at least one copy of a nucleotide sequence encoding a target sequence of a microRNA expressed in the liver, as defined in SEQ ID NO: 12 or 14-23, and at least one copy of a nucleotide sequence encoding a target sequence of a microRNA expressed in the heart, as defined in SEQ ID NO: 13 or 23-30, are present in the viral expression construct of the invention. In a further embodiment, two, three, four, five, six, seven or eight copies of a nucleotide sequence encoding a target sequence of a microRNA expressed in the liver, as defined in SEQ ID NO: 12 or 14-23, and two, three, four, five, six, seven or eight copies of a nucleotide sequence encoding a target sequence of a microRNA expressed in the heart, as defined in SEQ ID NO: 13 or 23-30, are present in the viral expression construct of the invention. In a further embodiment one, two, three, four, five, six, seven or eight copies of a nucleotide sequence encoding miRT122a (SEQ ID NO: 12) and one, two, three, four, five, six, seven or eight copies nucleotide sequence encoding miRT1 (SEQ ID NO: 13) are combined in the viral expression construct of the invention. In a further embodiment, four copies of a nucleotide sequence encoding miRT122a (SEQ ID NO: 12) and four copies of nucleotide sequence encoding miRT1 (SEQ ID NO: 13) are combined in the viral expression construct of the invention.

The definition “promoter”, “liver-specific promoter”, “adipose tissue-specific promoter”, “ubiquitous promoter”, “skeletal muscle promoter” has been provided in the part of the description entitled “general definitions”.

A preferred ubiquitous promoter is a CAG promoter.

In the context of the invention, a nucleotide sequence of a CAG promoter may be replaced by a nucleotide sequence comprising a nucleotide sequence that has at least 60% sequence identity or similarity with SEQ ID NO: 44. A preferred nucleotide sequence has at least 60%, 65%, 70%, 75%, 80%, 85%, 90%, 95%, 97%, 98%, 99%, 100% identity with SEQ ID NO: 44. Identity may be assessed over the whole SEQ ID NO or over part thereof as explained in the part of the description entitled “general definitions”.

Another preferred ubiquitous promoter is a cytomegalovirus (CMV) promoter.

In the context of the invention, a nucleotide sequence of a CMV promoter may be replaced by a nucleotide sequence comprising a nucleotide sequence that has at least 60% sequence identity or similarity with SEQ ID NO: 45. A preferred nucleotide sequence has at least 60%, 65%, 70%, 75%, 80%, 85%, 90%, 95%, 97%, 98%, 99%, 100% identity with SEQ ID NO: 45. Identity may be assessed over the whole SEQ ID NO or over part thereof as explained in the part of the description entitled “general definitions”.

Preferably said CMV promoter is used together with an intronic sequence. In this context an intronic sequence may be replaced by a nucleotide sequence comprising a nucleotide sequence that has at least 60% sequence identity or similarity with SEQ ID NO: 43. A preferred nucleotide sequence has at least 60%, 65%, 70%, 75%, 80%, 85%, 90%, 95%, 97%, 98%, 99%, 100% identity with SEQ ID NO: 43. Identity may be assessed over the whole SEQ ID NO or over part thereof as explained in the part of the description entitled “general definitions”.

A preferred liver-specific promoter is a human al-antitrypsin (hAAT) promoter.

In the context of the invention, a nucleotide sequence of a hAAT promoter may be replaced by a nucleotide sequence comprising a nucleotide sequence that has at least 60% sequence identity or similarity with SEQ ID NO: 47. A preferred nucleotide sequence has at least 60%, 65%, 70%, 75%, 80%, 85%, 90%, 95%, 97%, 98%, 99%, 100% identity with SEQ ID NO: 47. Identity may be assessed over the whole SEQ ID NO or over part thereof as explained in the part of the description entitled “general definitions”.

Preferably said hAAT promoter is used together with an intronic sequence. A preferred intronic sequence is a hepatocyte control region (HCR) enhancer from apolipoprotein E. A most preferred intronic sequence is the HCR enhancer from apolipoprotein E as defined in SEQ ID NO: 53. In this context an intronic sequence may be replaced by a nucleotide sequence comprising a nucleotide sequence that has at least 60% sequence identity or similarity with SEQ ID NO: 53. A preferred nucleotide sequence has at least 60%, 65%, 70%, 75%, 80%, 85%, 90%, 95%, 97%, 98%, 99%, 100% identity with SEQ ID NO: 53. Identity may be assessed over the whole SEQ ID NO or over part thereof as explained in the part of the description entitled “general definitions”. In an embodiment, said hAAT promoter is used together with one, two, three, four or five copies of an intronic sequence. In a preferred embodiment, said hAAT promoter is used together with one, two, three, four or five copies of the HCR enhancer from apolipoprotein E as defined in SEQ ID NO: 53.

Other liver-specific promoters are the albumin promoter, the major urinary protein promoter, the phosphoenolpyruvate carboxykinase (PEPCK) promoter, the liver-enriched protein activator promoter, the transthyretin promoter, the thyroxine binding globulin promoter, the apolipoprotein Al promoter, the liver fatty acid binding protein promoter and the phenylalanine hydroxylase promoter.

Adipose tissue-specific promoters are the adipocyte protein 2 (aP2, also known as fatty acid binding protein 4 (FABP4)) promoter, the PPARy promoter, the adiponectin promoter, the phosphoenolpyruvate carboxykinase (PEPCK) promoter, the promoter derived from human aromatase cytochrome p450 (p450arom) the mini/aP2 promoter (composed of the adipose-specific aP2 enhancer and the basal aP2 promoter), the uncoupling protein 1 (UCP1) promoter, the mini/UCPI promoter (composed of the adipose-specific UCP1 enhancer and the basal UCP1 promoter), the adipsin promoter, the leptin promoter, or the Foxa-2 promoter. Preferred adipose tissue-specific promoters are the mini/aP2 promoter (SEQ ID NO: 54) and the mini/UCP1 promoter (SEQ ID NO 55). In this context an adipose tissue-specific promoter sequence may be replaced by a nucleotide sequence comprising a nucleotide sequence that has at least 60% sequence identity or similarity with SEQ ID NO: 53 or SEQ ID NO: 54. A preferred nucleotide sequence has at least 60%, 65%, 70%, 75%, 80%, 85%, 90%, 95%, 97%, 98%, 99%, 100% identity with SEQ ID NO: 53 or SEQ ID NO: 54. Identity may be assessed over the whole SEQ ID NO or over part thereof as explained in the part of the description entitled “general definitions”.

Preferred skeletal muscle promoters are the myosin light-chain promoter, the myosin heavy-chain promoter, the desmin promoter, the muscle creatine kinase (MCK) promoter, the smooth muscle alpha-actin promoter, the CK6 promoter, the Unc-45 Myosin Chaperone B promoter, the basal MCK promoter in combination with copies of the MCK enhancer, the Enh358MCK promoter (combination of the MCK enhancer with the 358 bp proximal promoter of the MCK gene). A most preferred skeletal muscle promoter is the C5-12 promoter as defined in SEQ ID NO: 56. In this context a skeletal muscle promoter sequence may be replaced by a nucleotide sequence comprising a nucleotide sequence that has at least 60% sequence identity or similarity with SEQ ID NO: 56. A preferred nucleotide sequence has at least 60%, 65%, 70%, 75%, 80%, 85%, 90%, 95%, 97%, 98%, 99%, 100% identity with SEQ ID NO: 56. Identity may be assessed over the whole SEQ ID NO or over part thereof as explained in the part of the description entitled “general definitions”.

A promoter as used herein (especially when the promoter sequence is defined as having a minimal identity percentage with a given SEQ ID NO) should exert at least an activity of a promoter as known to the skilled person. Please be referred to the part of the description entitled “general definitions” for a definition of such activity. Preferably a promoter defined as having a minimal identity percentage with a given SEQ ID NO should control transcription of the nucleotide sequence it is operably linked thereto (i.e. a nucleotide sequence encoding a FGF21) as assessed in an assay known to the skilled person. In the context of the invention, said promoter is operatively linked to the FGF21 nucleotide sequence defined above. In one embodiment, the promoter is cell-specific and/or tissue-specific, preferably specific for liver, adipose tissue and/or skeletal muscle.

Several viral expression constructs are therefore encompassed by the present invention:
A viral expression construct comprising a nucleotide sequence encoding a FGF21 suitable for expression in a mammal and comprising element a),
A viral expression construct comprising a nucleotide sequence encoding a FGF21 suitable for expression in a mammal and comprising element b),
A viral expression construct comprising a nucleotide sequence encoding a FGF21 suitable for expression in a mammal and comprising element c),
A viral expression construct comprising a nucleotide sequence encoding a FGF21 suitable for expression in a mammal and comprising element d),
A viral expression construct comprising a nucleotide sequence encoding a FGF21 suitable for expression in a mammal and comprising element e),
A viral expression construct comprising a nucleotide sequence encoding a FGF21 suitable for expression in a mammal and comprising element b) and a nucleotide sequence of element c),
A viral expression construct comprising a nucleotide sequence encoding a FGF21 suitable for expression in a mammal and comprising element e) and a nucleotide sequence of element c).

In an embodiment there is provided a viral expression construct as described herein, wherein the liver-specific promoter is the human al-antitrypsin (hAAT) promoter and/or the adipose tissue-specific promoter is the mini/ap2 promoter and/or the mini/UCP1 promoter and/or the skeletal muscle promoter is the C5-12 promoter and/or the ubiquitous promoter is the cytomegalovirus (CMV) promoter and/or the CAG promoter.

In an embodiment, a viral expression construct is encompassed comprising a nucleotide sequence encoding FGF21 suitable for expression in a mammal and element a), wherein the liver-specific promoter is a hAAT promoter (SEQ ID NO: 47).

In a preferred embodiment, a viral expression construct is encompassed comprising a nucleotide sequence encoding FGF21 suitable for expression in a mammal and element a) wherein said construct is AAV8-hAAT-moFGF21. This construct for example contains a viral expression construct as depicted in FIG. 6A: ITR2-hAAT-moFGF21-polyA-ITR2; the sequence of this expression construct is comprised in SEQ ID NO:34. For this construct, Example 3 surprisingly reveals high and stable liver-specific expression after intravenous administration. Expression was shown to be stable for up to 1 year (Example 12). Extensive beneficial therapeutic effects for the reversion and treatment of obesity and diabetes are shown in ob/ob mice (Examples 3 and 11), high fat diet (HFD)-fed mice (Examples 4, 12-14) and old HFD-fed mice (Examples 5, 12-14). Examples 11 and 16 also reveal marked improvement of hepatic steatosis, hepatic inflammation and hepatic fibrosis. Example 15 shows improvement of the inflammation of WAT associated to obesity. Example 17 indicates the long-term safety of the therapy. Example 18 reveals a beneficial effect in preventing liver tumors. Example 19 shows therapeutic potential in a model for type I diabetes.

In an embodiment, a viral expression construct is encompassed comprising a nucleotide sequence encoding FGF21 suitable for expression in a mammal and element b), wherein the adipose tissue-specific promoter is a mini/aP2 promoter (SEQ ID NO: 54) and/or a mini/UCP1 promoter (SEQ ID NO 55).

In an embodiment, a viral expression construct is encompassed comprising a nucleotide sequence encoding FGF21 suitable for expression in a mammal and element c), wherein the ubiquitous promoter is a CAG Promoter (SEQ ID NO: 44) and wherein the at least one nucleotide sequence encoding a target sequence of a microRNA expressed in the liver is selected from the group consisting of SEQ ID NO: 12 or 14-23 and the at least one nucleotide sequence encoding a target sequence of a microRNA expressed in the heart is selected from the group consisting of SEQ ID NO: 13 or 23-30.

In a preferred embodiment, a viral expression construct is encompassed comprising a nucleotide sequence encoding FGF21 suitable for expression in a mammal and element c) wherein said construct is AAV9-CAG-moFGF21-dmiRT or AAV8-CAG-moFGF21-dmiRT. The notations dmiRT and doublemiRT are equivalent. These constructs for example contain a viral expression construct as depicted in FIG. 1A: ITR2-CAG-moFGF21-4x miRT122a-4x miRT1-polyA-ITR2; the sequence of this expression construct is comprised in SEQ ID NO:32.

For these constructs, Examples 1-2 surprisingly reveal high and stable adipose-specific expression after intra-eWAT administration. Extensive beneficial therapeutic effects for the prevention, reversion and treatment of obesity and diabetes are shown in normal mice (Example 1) and ob/ob mice (Examples 2 and 10). Example 10 also reveals improvement of hepatic steatosis.

In an embodiment, a viral expression construct is encompassed comprising a nucleotide sequence encoding FGF21 suitable for expression in a mammal and element d), wherein the skeletal muscle promoter is a C5-12 promoter (SEQ ID NO: 56).

In an embodiment, a viral expression construct is encompassed comprising a nucleotide sequence encoding FGF21 suitable for expression in a mammal and element e), wherein the ubiquitous promoter is a CMV promoter (SEQ ID NO: 45) and the AAV serotype is AAV1.

In a preferred embodiment, a viral expression construct is encompassed comprising a nucleotide sequence encoding FGF21 suitable for expression in a mammal and element e) wherein said construct is AAV1-CMV-moFGF21. This construct for example contains a viral expression construct as depicted in FIG. 11A: ITR2-CMV-moFGF21-polyA-ITR2; the sequence of this expression construct is comprised in SEQ ID NO:36. For this construct, Example 20 reveals high and stable skeletal muscle-specific expression after intramuscular administration. Extensive beneficial therapeutic effects for the prevention, reversion and treatment of obesity and diabetes are shown in HFD-fed mice (Examples 6 and 21). Example 20 reveals a beneficial effect in extending healthy lifespan by preventing obesity and diabetes.

In an embodiment, a viral expression construct is encompassed comprising a nucleotide sequence encoding FGF21 suitable for expression in a mammal and element b) and a nucleotide sequence of element c), wherein the adipose tissue-specific promoter is a mini/aP2 promoter (SEQ ID NO: 54) and/or the mini/UCP1 promoter (SEQ ID NO 55).

In an embodiment, a viral expression construct is encompassed comprising a nucleotide sequence encoding FGF21 suitable for expression in a mammal and element e) and a nucleotide sequence of element c), wherein the ubiquitous promoter is a CMV promoter (SEQ ID NO: 45) and the AAV serotype is AAV1.

All constructs of the invention are more attractive than the ones of the prior art, such as the one disclosed in Zhang et al., EBioMedicine 15 (2017) 173-183, especially the ones comprising element a) which is a liver-specific promoter, preferably hAAT, and/or element c) which is a combination of an ubiquitous promoter and at least one nucleotide sequence encoding a target sequence of a microRNA expressed in the liver, preferably miRT122a, and at least one nucleotide sequence encoding a target sequence of a microRNA expressed in the heart, preferably miRT1, wherein said combination enables specific expression in adipose tissue, and/or element c) which is a combination of an ubiquitous promoter, preferably CMV, and an adeno-associated virus (AAV) vector sequence, preferably AAV1, wherein said combination enables specific expression in skeletal muscle. Zhang et al. discloses wild type murine FGF21 coding sequence under the control of the elongation factor 1a (EF1a) promoter (EF1a-mFGF21) (Zhang et al., EBioMedicine 15 (2017) 173-183). This construct was compared with constructs of the invention in Examples 23 and 24. In all the in vitro and in vivo experiments, all expression cassettes and AAV vectors of the invention mediated higher expression of FGF21 in the target tissue or cell type and lower expression of FGF21 in off-target tissues, demonstrating higher efficiency of the expression cassettes and AAV vectors of the invention as well as higher tissue-specificity. In addition, constructs CMV-moFGF21 and CAG-moFGF21-double miRT also mediated higher protein production and secretion to the culture media in HEK293 cells in comparison to EF1a-mFGF21. Moreover, hAAT-moFGF21 and AAV8-hAAT-moFGF21 also mediated higher secretion of FGF21 to the bloodstream than EF1a-mFGF21 and AAV8-EF1a-mFGF21.

Additional sequences may be present in the viral expression construct of the invention as explained in detail in the part of the description entitled “general definitions”. Preferred additional sequences include inverted terminal repeats (ITRs), a SV40 polyadenylation signal (SEQ ID NO: 50), a rabbit β-globin polyadenylation signal (SEQ ID NO: 51), a CMV enhancer sequence (SEQ ID NO: 46) and a HCR enhancer from apolipoprotein E (SEQ ID NO: 53). Within the context of the invention, “ITRs” is intended to encompass one 5′ITR and one 3′ITR, each being derived from the genome of an AAV. Preferred ITRs are from AAV2 and are represented by SEQ ID NO: 48 (5′ ITR) and SEQ ID NO: 49 (3′ ITR). Within the context of the invention, it is encompassed to use the CMV enhancer sequence (SEQ ID NO: 46) and the CMV promoter sequence (SEQ ID NO: 45) as two separate sequences or as a single sequence (SEQ ID NO: 52).

Each of these additional sequences may be present in the viral expression construct of the invention (see for example as depicted in FIGS. 1, 2, 3, 4, 5, 6, 7, 8 and 9 and also as depicted in FIGS. 11, 31 and 32).

In an embodiment, the viral expression construct comprising a nucleotide sequence encoding FGF21 suitable for expression in a mammal and at least one of elements a) and/or b) and/or c) and/or d) and/or e) as earlier defined further comprises:
ITRs that flank the expression cassette of said construct,
SV40 or rabbit β-globin polyadenylation signals that are located at the 3′ of the nucleotide sequence encoding the FGF21 and/or
a CMV enhancer sequence or a HCR enhancer sequence that is located at the 5′ of the nucleotide sequence encoding the FGF21.

In a preferred embodiment, the viral expression construct comprising a nucleotide sequence encoding FGF21 suitable for expression in a mammal and at least one of elements a) and/or b) and/or c) and/or d) and/or e) as earlier defined further comprises ITRs that flank the expression cassette of said construct and optionally
SV40 or rabbit β-globin polyadenylation signals that are located at the 3′ of the nucleotide sequence encoding the FGF21 and/or
a CMV enhancer sequence or a HCR enhancer sequence that is located at the 5′ of the nucleotide sequence encoding the FGF21.

These sequences were used in the experimental part in some of the constructs identified herein.

Therefore, in one embodiment, for each of these preferred viral expression constructs defined above an additional sequence may be present selected from the group consisting of: ITRs, SV40 polyadenylation signal, rabbit β-globin polyadenylation signal, CMV enhancer sequence, HCR enhancer sequence from apolipoprotein E.

In a preferred embodiment, the viral expression construct comprises a nucleotide sequence encoding FGF21 suitable for expression in a mammal and at least one of elements a) and/or b) and/or c) and/or d) and/or e), wherein an additional sequence is present which is selected from the group consisting of: ITRs, SV40 polyadenylation signal, rabbit β-globin polyadenylation signal, CMV enhancer sequence, HCR enhancer sequence. Preferred ITRs are those of AAV2 which are represented by SEQ ID NO: 48 (5′ ITR) and SEQ ID NO: 49 (3′ ITR).

Preferred viral expression constructs comprise elements a) and/or b) and/or c) and/or d) and/or e) and are such that the expression cassette is flanked by a 5′ITR and a 3′ITR. Other preferred viral expression constructs comprise elements a) and/or b) and/or c) and/or d) and/or e) and are such that the expression cassette is flanked by a 5′ITR and a 3′ITR. In addition, SV40 polyadenylation signals are present.

Other preferred viral expression constructs comprise elements a) and/or b) and/or c) and/or d) and/or e) and are such that the expression cassette is flanked by a 5′ITR and a 3′ITR. In addition, rabbit β-globin polyadenylation signals are present.

Other preferred viral expression constructs comprise elements a) and/or b) and/or c) and/or d) and/or e) and are such that the expression cassette is flanked by a 5′ITR and a 3′ITR. In addition, CMV enhancer sequence is present.

Other preferred viral expression constructs comprise elements a) and/or b) and/or c) and/or d) and/or e) and are such that the expression cassette is flanked by a 5′ITR and a 3′ITR. In addition, HCR enhancer sequence from apolipoprotein E is present.

Most preferred designed viral expression constructs include:
Construct B (represented by a nucleotide sequence comprising SEQ ID NO: 32),
Construct D (represented by a nucleotide sequence comprising SEQ ID NO: 34),
Construct F (represented by a nucleotide sequence comprising SEQ ID NO: 36),
Construct G (represented by a nucleotide sequence comprising SEQ ID NO: 37),
Construct H (represented by a nucleotide sequence comprising SEQ ID NO: 38),
Construct I (represented by a nucleotide sequence comprising SEQ ID NO: 39),
Construct J (represented by a nucleotide sequence comprising SEQ ID NO: 40),
Construct K (represented by a nucleotide sequence comprising SEQ ID NO: 41).
Construct L (represented by a nucleotide sequence comprising SEQ ID NO: 42).

As the skilled person will understand, each of these viral expression constructs already comprise two ITRs from AAV2 (i.e. SEQ ID NO: 48 (5′ ITR) and SEQ ID NO: 49 (3′ ITR)).

Constructs B and G comprise a rabbit β-globin polyadenylation signal. Construct F comprises a SV40 polyadenylation signal, a CMV enhancer sequence and a nucleotide sequence of a chimeric intron (composed of introns from human β-globin and immunoglobulin heavy chain genes). Constructs D, H-L comprise a SV40 polyadenylation signal, a HCR enhancer sequence and a nucleotide sequence of chimeric intron (composed of introns from human β-globin and immunoglobulin heavy chain genes).

As explained in the general part entitled “general definitions”, throughout this application, each time one refers to a specific nucleotide sequence SEQ ID NO (take SEQ ID NO: A, B or C) representing the preferred constructs designed herein, one may replace it by:
i. a nucleotide sequence comprising a nucleotide sequence that has at least 60% sequence identity or similarity with SEQ ID NO: A, B or C;
ii. a nucleotide sequence the sequence of which differs from the sequence of a nucleic acid molecule of (i) due to the degeneracy of the genetic code.

Each nucleotide sequence described herein by virtue of its identity percentage (at least 60%) with a given nucleotide sequence respectively has in a further preferred embodiment an identity of at least 65%, 70%, 75%, 80%, 85%, 90%, 95%, 97%, 98%, 99% or more identity with the given nucleotide respectively. In a preferred embodiment, sequence identity is determined by comparing the whole length of the sequences as identified herein. Unless otherwise indicated herein, identity with a given SEQ ID NO means identity or similarity based on the full length of said sequence (i.e. over its whole length or as a whole).

A construct defined by its minimum identity (i.e. at least 60%) to a given SEQ ID NO as identified above is encompassed within the scope of the invention when this construct or a viral expression construct or a viral vector comprising this construct or a composition comprising this construct or vector is able to induce the expression of FGF21 in a cell, preferably in a liver cell, cell of adipose tissue or in a cell of skeletal muscle. The expression of FGF21 could be assessed using techniques known to the skilled person. In a preferred embodiment, said expression is assessed as carried out in the experimental part.

In a preferred embodiment, a viral expression construct is such that the construct is represented by a nucleotide sequence comprising SEQ ID NO: 4, 5, 6, 7, 8, 9, 10 or 11 or a sequence having at least 60% identity with SEQ ID NO: 4, 5, 6, 7, 8, 9, 10 or 11 or a sequence having at least 60%, 65%, 70%, 75%, 80%, 85%, 90%, 95%, 97%, 98%, 99%, 100% identity with SEQ ID NO: 4, 5, 6, 7, 8, 9, 10 or 11.

### Viral Vector

In a further aspect, there is provided a viral vector comprising a viral expression construct as defined above, wherein said viral vector is an adenovirus vector, an adeno-associated virus vector, a retrovirus vector or a lentivirus vector, preferably an adeno-associated virus vector selected from the group consisting of an adeno-associated virus 1 (AAV1) vector, an adeno-associated virus 8 (AAV8) vector, and an adeno-associated virus 9 (AAV9) vector.

A “viral vector” and an “adeno-associated virus vector (AAV vector)” are further defined in the part of the description entitled “general definitions”.

In an embodiment, an AAV vector is used comprising each of the elements defined earlier herein and a recombinant AAV (rAAV) based genome comprising a ITR or a part thereof. Preferred ITRs are those of AAV2 which are represented by SEQ ID NO: 48 (5′ ITR) and SEQ ID NO: 49 (3′ ITR).

Preferably, said AAV vector is an AAV1 vector, an AAV8 vector or an AAV9 vector.

A viral expression construct and a viral vector of the invention are preferably for use as a medicament. The medicament is preferably for preventing, delaying, curing, reverting and/or treating a metabolic disorder, preferably a diabetes and/or obesity. Diabetes may be Type 1 Diabetes, Type 2 Diabetes or Monogenic Diabetes. In another preferred embodiment, the medicament is for preventing, delaying, curing, reverting and/or treating liver inflammation and/or fibrosis. In yet another preferred embodiment, the medicament is for extending healthy lifespan, preferably by preventing, delaying, curing, reverting and/or treating a metabolic disorder associated with aging, preferably a diabetes and/or obesity. In yet another preferred embodiment, the medicament is for preventing, delaying, curing, reverting and/or treating cancer, preferably liver cancer.

The subject treated may be a higher mammal, e.g. cats, rodents, (preferably mice, rats, gerbils and guinea pigs, and more preferably mice and rats), or dogs, or human beings.

### Nucleic Acid Molecule

In a further aspect, there is provided a nucleic acid molecule suitable for expression in a mammal and represented by a mammalian codon optimized nucleotide sequence encoding a FGF21 to be expressed in liver, adipose tissue and/or skeletal muscle.

The definition of “codon optimization” has been provided in the part of the description entitled “general definitions”.

In an embodiment, a nucleic acid molecule is encompassed as described above, wherein the nucleotide sequence has at least 60% sequence identity with the nucleotide sequence of SEQ ID NO: 4, 5, 6, 7, 8, 9, 10 or 11. A preferred nucleotide sequence has at least 65%, 70%, 75%, 80%, 85%, 90%, 95%, 97%, 98%, 99%, 100% identity with SEQ ID NO: 4, 5, 6, 7, 8, 9, 10 or 11.

### Composition

In a further aspect there is provided a composition comprising a viral expression construct as defined above and/or a viral vector as defined above and/or a nucleic acid molecule as defined above, together with one or more pharmaceutically acceptable excipients or vehicles.

This composition is preferably called a gene therapy composition. Preferably, the composition is a pharmaceutical composition said pharmaceutical composition comprising a pharmaceutically acceptable carrier, adjuvant, diluents, solubilizer, filler, preservative and/or excipient.

Such pharmaceutically acceptable carrier, filler, preservative, solubilizer, diluent and/or excipient may for instance be found in Remington: The Science and Practice of Pharmacy, 20th Edition. Baltimore, MD: Lippincott Williams & Wilkins, 2000.

In a preferred embodiment, said composition is for use as a medicament, preferably for preventing, delaying, curing, reverting and/or treating a metabolic disorder, preferably a diabetes and/or obesity. Diabetes may be Type 1 Diabetes, Type 2 Diabetes or

Monogenic Diabetes. In another preferred embodiment, the medicament is for preventing, delaying, curing, reverting and/or treating liver inflammation and/or fibrosis. In yet another preferred embodiment, the medicament is for extending healthy lifespan, preferably by preventing, delaying, curing, reverting and/or treating a metabolic disorder associated with aging, preferably a diabetes and/or obesity. In yet another preferred embodiment, the medicament is for preventing, delaying, curing, reverting and/or treating cancer, preferably liver cancer. The subject treated may be a higher mammal, e.g. cats, rodent, (preferably mice, rats, gerbils and guinea pigs, and more preferably mice and rats), or dogs, or a human being.

Said viral expression construct, viral vector and/or nucleic acid molecule and/or composition are preferably said to be able to be used for preventing, delaying, reverting, curing and/or treating a metabolic disorder, preferably a diabetes and/or obesity, when said viral expression construct, viral vector and/or nucleic acid molecule and/or composition are able to exhibit an anti-diabetes effect and/or an anti-obesity effect.

Said viral expression construct, viral vector and/or nucleic acid molecule and/or composition are preferably said to be able to be used for preventing, delaying, curing, reverting and/or treating liver inflammation and/or fibrosis, when said viral expression construct, viral vector and/or nucleic acid molecule and/or composition are able to exhibit an anti-fibrotic effect.

Said viral expression construct, viral vector and/or nucleic acid molecule and/or composition are preferably said to be able to be used for extending healthy lifespan, preferably by preventing, delaying, curing, reverting and/or treating a metabolic disorder associated with aging, preferably a diabetes and/or obesity, when said viral expression construct, viral vector and/or nucleic acid molecule and/or composition are able to exhibit an anti-diabetes effect and/or an anti-obesity effect during aging.

Said viral expression construct, viral vector and/or nucleic acid molecule and/or composition are preferably said to be able to be used for preventing, delaying, curing, reverting and/or treating cancer, preferably liver cancer, when said viral expression construct, viral vector and/or nucleic acid molecule and/or composition are able to exhibit an anti-cancer effect.

An anti-diabetes effect may be reached when glucose disposal in blood is increased and/or when glucose tolerance is improved and/or when insulin sensitivity is increased. This could be assessed using techniques known to the skilled person or as done in the experimental part, preferably as assessed in example 8 or 9. In this context, “increase” (respectively “improvement”) means at least a detectable increase (respectively a detectable improvement) using an assay known to the skilled person or using assays as carried out in the experimental part, such as measurement of glycaemia, insulinemia and/or performance of an insulin tolerance test and/or of a glucose tolerance test. The increase may be an increase of at least 5%, at least 10%, at least 20%, at least 30%, at least 40%, at least 50%, at least 60%, at least 70%, at least 80%, at least 90% or at least 100% using assays such as the measurement of glycaemia, insulinemia and/or performance of an insulin tolerance test and/or of a glucose tolerance test.

An anti-obesity effect may be reached when body weight, body weight gain and/or body fat percentage is decreased. An anti-obesity effect may also be reached when body mass index (BMI), waist circumference, waist-to-hip ratio (WHR) and/or waist-to-height ratio (WHtR) is decreased. This could be assessed using techniques known to the skilled person or as done in the experimental part. In this context, “decrease” (respectively “improvement”) means at least a detectable decrease (respectively a detectable improvement) using an assay known to the skilled person or using assays as carried out in the experimental part. Anti-obesity effects include both prevention of obesity and reversion of obesity, as evaluated by measurement of body weight of the individual, the BMI and/or weight of the tissues.

An anti-inflammatory effect in the liver may be reached by a decrease in macrophage infiltration, decreased pro-inflammatory cytokines. This could be assessed using techniques known to the skilled person or as done in the experimental part. In this context, “decrease” (respectively “improvement”) means at least a detectable decrease (respectively a detectable improvement) using an assay known to the skilled person or using assays as carried out in the experimental part.

An anti-fibrotic effect in the liver may be reached by a decrease in deposited extracellular matrix proteins, blood markers (e.g. including N-terminal propeptide of type III collagen, hyaluronic acid, tissue inhibitor of metalloproteinase type 1 (TIMP-1), YKL-40, serum glutamate oxaloacetate transaminase (SGOT), serum glutamate pyruvic transaminase (SGPT) levels in the plasma). An anti-fibrotic effect may also be reached by an improvement in a fibrosis scoring system such as Metavir or Ishak. This could be assessed using techniques known to the skilled person or as done in the experimental part. In this context, “decrease” (respectively “improvement”) means at least a detectable decrease (respectively a detectable improvement) using an assay known to the skilled person or using assays as carried out in the experimental part.

A healthy lifespan-extending effect may be reached when an anti-diabetes and/or anti-obesity effect as defined earlier herein is used to prevent, delay, cure, reverse or treat the onset or progression of a metabolic disorder associated with aging, preferably of a diabetes and/or obesity. A healthy lifespan-extending effect may also be reached by an increase in the healthy lifespan, wherein symptoms associated with metabolic disorders, preferably of a diabetes and/or obesity, are absent or reduced. A healthy-lifespan extending effect may also be reached by improved coordination and balance (assessed by Rota-Rod test), memory (assessed by Object Recognition Test), and/or neuromuscular coordination (assessed by Tightrope Test), decreased mitochondrial and metabolic deterioration (assessed by measurement of expression levels of genes involved in metabolism and mitochondrial function such as PGC-1alpha, ATP synthase and ERRalpha). This could be assessed using techniques known to the skilled person or as done in the experimental part. In this context, “increase” (respectively “improvement”) means at least a detectable increase (respectively a detectable improvement) using an assay known to the skilled person or using assays as carried out in the experimental part. An anti-cancer effect may be reached by a decrease in the cumulative incidence of cancer over the lifetime. This could be assessed using techniques known to the skilled person or as done in the experimental part. In this context, “decrease” (respectively “improvement”) means at least a detectable decrease (respectively a detectable improvement) using an assay known to the skilled person or using assays as carried out in the experimental part.

An anti-diabetes effect and/or anti-obesity effect may also be observed when the progression of a typical symptom (e.g. insulitis, beta cell loss, increase of body weight) has been slowed down as assessed by a physician. A decrease of a typical symptom may mean a slow down in progression of symptom development or a complete disappearance of symptoms. Symptoms, and thus also a decrease in symptoms, can be assessed using a variety of methods, to a large extent the same methods as used in diagnosis of diabetes and/or obesity, including clinical examination and routine laboratory tests. Such methods include both macroscopic and microscopic methods, as well as molecular methods, X-rays, biochemical, immunohistochemical and others.

An anti-inflammatory effect in the liver may also be observed when the progression of a typical symptom (e.g. fatigue, flu-like symptoms, dark urine, pale stool, abdominal pain, loss of appetite, unexplained weight loss, jaundice) has been slowed down as assessed by a physician. A decrease of a typical symptom may mean a slow down in progression of symptom development or a complete disappearance of symptoms. Symptoms, and thus also a decrease in symptoms, can be assessed using a variety of methods, to a large extent the same methods as used in diagnosis of liver fibrosis, including clinical examination and routine laboratory tests. Such methods include both macroscopic and microscopic methods, as well as molecular methods, imaging methods (elastography, X-rays, MRI, CT, ultrasonography, angiography), biochemical, immunohistochemical and others.

An anti-fibrotic effect in the liver may also be observed when the progression of a typical symptom (e.g. liver stiffness, jaundice, appetite loss, difficulty thinking clearly, fluid buildup in the legs or stomach, nausea, unexplained weight loss, weakness) has been slowed down as assessed by a physician. A decrease of a typical symptom may mean a slow down in progression of symptom development or a complete disappearance of symptoms. Symptoms, and thus also a decrease in symptoms, can be assessed using a variety of methods, to a large extent the same methods as used in diagnosis of liver fibrosis, including clinical examination and routine laboratory tests. Such methods include both macroscopic and microscopic methods, as well as molecular methods, imaging methods (elastography, X-rays, MRI, CT, ultrasonography, angiography), biochemical, immunohistochemical and others.

An healthy lifespan-extending effect may also be observed when the progression of a typical symptom of metabolic disorders associated with aging (e.g. insulin resistance, glucose intolerance, increase of body weight) has been slowed down as assessed by a physician. A decrease of a typical symptom may mean a slow down in progression of symptom development or a complete disappearance of symptoms. Symptoms, and thus also a decrease in symptoms, can be assessed using a variety of methods, to a large extent the same methods as used in diagnosis of diabetes and/or obesity, including clinical examination and routine laboratory tests. Such methods include both macroscopic and microscopic methods, as well as molecular methods, X-rays, biochemical, immunohistochemical and others.

An anti-cancer effect may also be observed when the progression of a typical symptom (e.g. tumor size, unexplained weight loss, loss of appetite, feeling very full after a small meal, nausea or vomiting, enlarged liver, enlarged spleen, pain in the abdomen or near the right shoulder blade, swelling or fluid build-up in the abdomen, itching, jaundice) has been slowed down as assessed by a physician. A decrease of a typical symptom may mean a slow down in progression of symptom development or a complete disappearance of symptoms. Symptoms, and thus also a decrease in symptoms, can be assessed using a variety of methods, to a large extent the same methods as used in diagnosis of cancer, including clinical examination and routine laboratory tests. Such methods include both macroscopic and microscopic methods, as well as molecular methods, imaging methods (X-rays, MRI, CT, ultrasonography, angiography), biochemical, immunohistochemical and others.

A medicament as defined herein (viral expression construct, viral vector, nucleic acid molecule, composition) is preferably able to alleviate one symptom or one characteristic of a patient or of a cell, tissue or organ of said patient if after at least one week, one month, six months, one year or more of treatment using a viral expression construct and/or a viral vector and/or a nucleic acid molecule and/or a composition of the invention, said symptom or characteristic has decreased (e.g. is no longer detectable or has slowed down), as defined above.

A viral expression construct and/or a viral vector and/or a nucleic acid molecule and/or a composition as defined herein for use according to the invention may be suitable for administration to a cell, tissue and/or an organ in vivo of individuals affected by or at risk of developing a metabolic disorder, such as a diabetes and/or obesity, liver inflammation and/or fibrosis, a metabolic disorder associated with aging, and/or cancer, and may be administered in vivo, ex vivo or in vitro. Said viral expression construct and/or a viral vector and/or a nucleic acid molecule and/or composition may be directly or indirectly administrated to a cell, tissue and/or an organ in vivo of an individual affected by or at risk of developing a metabolic disorder, such as a diabetes and/or obesity, liver inflammation and/or fibrosis, a metabolic disorder associated with aging, and/or cancer, and may be administered directly or indirectly in vivo, ex vivo or in vitro. An administration mode may be intravenous, subcutaneous, intramuscular, intrathecal, intraarticular, intraventricular, intraperitoneal, intra-adipose tissue, via inhalation, oral, intranasal, intrahepatic, intrasplanchnic, intra-ocular, intra-car, topic administration and/or via retrograde intraductal pancreatic administration. A preferred administration mode is intramuscular, intravenous or intra-adipose tissue, as described in the “General procedures to the Examples” as part of this application.

A viral expression construct and/or a viral vector and/or a nucleic acid molecule and/or a composition of the invention may be directly or indirectly administered using suitable means known in the art. Improvements in means for providing an individual or a cell, tissue, organ of said individual with a viral expression construct and/or a viral vector and/or a nucleic acid molecule and/or a composition of the invention are anticipated, considering the progress that has already thus far been achieved. Such future improvements may of course be incorporated to achieve the mentioned effect of the invention. A viral expression construct and/or a viral vector and/or a nucleic acid molecule and/or a composition can be delivered as is to an individual, a cell, tissue or organ of said individual. Depending on the disease or condition, a cell, tissue or organ of said individual may be as earlier defined herein. When administering a viral expression construct and/or a viral vector and/or a nucleic acid molecule and/or a composition of the invention, it is preferred that such viral expression construct and/or vector and/or nucleic acid and/or composition is dissolved in a solution that is compatible with the delivery method.

As encompassed herein, a therapeutically effective dose of a viral expression construct, vector, nucleic acid molecule and/or composition as mentioned above is preferably administered in a single and unique dose hence avoiding repeated periodical administration. More preferably, the single dose is administered to skeletal muscle, to adipose tissue or intravenously.

A further compound may be present in a composition of the invention. Said compound may help in delivery of the composition. Below is provided a list of suitable compounds: compounds capable of forming complexes, nanoparticles, micelles and/or liposomes that deliver each constituent as defined herein, complexed or trapped in a vesicle or liposome through a cell membrane. Many of these compounds are known in the art. Suitable compounds comprise polyethylenimine (PEI), or similar cationic polymers, including polypropyleneimine or polyethylenimine copolymers (PECs) and derivatives, synthetic amphiphiles (SAINT-18), lipofectin™, DOTAP.

Depending on their identity, the skilled person will know which type of formulation is the most appropriate for the composition as defined herein.

### Method/Use

In a further aspect there is provided a viral expression construct as defined above and/or a viral vector as defined above and/or a nucleic acid molecule as defined above and/or a composition as defined above, for use as a medicament.

In an embodiment, said viral expression construct and/or viral vector and/or nucleic acid molecule and/or composition is provided for use in the treatment and/or prevention of a metabolic disorder, preferably a diabetes and/or obesity. Complications of a metabolic disorder may also be encompassed.

In another embodiment, said viral expression construct and/or viral vector and/or nucleic acid molecule and/or composition is provided for use in the treatment and/or prevention of liver inflammation and/or fibrosis. Complications of liver inflammation and/or fibrosis may also be encompassed.

In yet another embodiment, said viral expression construct and/or viral vector and/or nucleic acid molecule and/or composition is provided for use in extending healthy lifespan, preferably by preventing, delaying, curing, reverting and/or treating a metabolic disorder associated with aging, preferably a diabetes and/or obesity.

In yet another embodiment, said viral expression construct and/or viral vector and/or nucleic acid molecule and/or composition is provided for use in the treatment and/or prevention of cancer, preferably liver cancer. Complications of a cancer may also be encompassed.

In a further aspect there is provided a method for preventing, delaying, reverting, curing and/or treating a metabolic disorder, preferably a diabetes and/or obesity and their complications, comprising the use of a viral expression construct as defined above and/or a viral vector as defined above and/or a nucleic acid molecule as defined above and/or a composition as defined above.

Such a method is preferably for alleviating one or more symptom(s) of a metabolic disorder, such as a diabetes and/or obesity, in an individual, in a cell, tissue or organ of said individual or alleviate one or more characteristic(s) or symptom(s) of a cell, tissue or organ of said individual, the method comprising administering to said individual a viral expression construct and/or a viral vector and/or a nucleic acid molecule and/or a composition as defined herein.

In a further aspect there is provided a method for preventing, delaying, reverting, curing and/or treating liver inflammation and/or fibrosis and its complications, comprising the use of a viral expression construct as defined above and/or a viral vector as defined above and/or a nucleic acid molecule as defined above and/or a composition as defined above. Such a method is preferably for alleviating one or more symptom(s) of liver inflammation and/or fibrosis, in an individual, in a cell, tissue or organ of said individual or alleviate one or more characteristic(s) or symptom(s) of a cell, tissue or organ of said individual, the method comprising administering to said individual a viral expression construct and/or a viral vector and/or a nucleic acid molecule and/or a composition as defined herein.

In a further aspect there is provided a method for extending healthy lifespan, preferably by preventing, delaying, curing, reverting and/or treating a metabolic disorder associated with aging, preferably a diabetes and/or obesity, comprising the use of a viral expression construct as defined above and/or a viral vector as defined above and/or a nucleic acid molecule as defined above and/or a composition as defined above.

Such a method is preferably for alleviating one or more symptom(s) of a metabolic disorder associated with aging, such as a diabetes and/or obesity, in an individual, in a cell, tissue or organ of said individual or alleviate one or more characteristic(s) or symptom(s) of a cell, tissue or organ of said individual, the method comprising administering to said individual a viral expression construct and/or a viral vector and/or a nucleic acid molecule and/or a composition as defined herein.

In a further aspect there is provided a method for preventing, delaying, reverting, curing and/or treating cancer, preferably liver cancer and its complications, comprising the use of a viral expression construct as defined above and/or a viral vector as defined above and/or a nucleic acid molecule as defined above and/or a composition as defined above. Such a method is preferably for alleviating one or more symptom(s) of cancer, such as liver cancer, in an individual, in a cell, tissue or organ of said individual or alleviate one or more characteristic(s) or symptom(s) of a cell, tissue or organ of said individual, the method comprising administering to said individual a viral expression construct and/or a viral vector and/or a nucleic acid molecule and/or a composition as defined herein.

In the context of the invention there is provided a use of a viral expression construct and/or a viral vector and/or a nucleic acid molecule and/or a composition as defined herein for the manufacture of a medicament for preventing, delaying, reverting, curing and/or treating a metabolic disorder, preferably a diabetes and/or obesity.

In the context of the invention there is provided a use of a viral expression construct and/or a viral vector and/or a nucleic acid molecule and/or a composition as defined herein for the manufacture of a medicament for preventing, delaying, curing, reverting and/or treating liver inflammation and/or fibrosis.

In the context of the invention there is provided a use of a viral expression construct and/or a viral vector and/or a nucleic acid molecule and/or a composition as defined herein for the manufacture of a medicament for extending healthy lifespan, preferably by preventing, delaying, curing, reverting and/or treating a metabolic disorder associated with aging, preferably a diabetes and/or obesity.

In the context of the invention there is provided a use of a viral expression construct and/or a viral vector and/or a nucleic acid molecule and/or a composition as defined herein for the manufacture of a medicament for preventing, delaying, reverting, curing and/or treating cancer, preferably liver cancer.

Metabolic disorders include metabolic syndrome, diabetes, obesity, obesity-related comorbidities, diabetes-related comorbidities, hyperglycemia, insulin resistance, glucose intolerance, hepatic steatosis, alcoholic liver diseases (ALD), non-alcoholic fatty liver disease (NAFLD), non-alcoholic steatohepatitis (NASH), coronary heart disease (CHD), hyperlipidemia, atherosclerosis, endocrinophaties, osteosarcopenic obesity syndrome (OSO), diabetic nephropaty, chronic kidney disease (CKD), cardiac hypertrophy, diabetic retinopathy, diabetic nephropathy, diabetic neuropathy, arthritis, sepsis, ocular neovascularization, neurodegeneration, dementia, and may also include depression, adenoma, carcinoma.

Diabetes includes prediabetes, hyperglycemia, Type 1 diabetes, Type 2 diabetes, maturity-onset diabetes of the young (MODY), monogenic diabetes, neonatal diabetes, gestational diabetes, brittle diabetes, idiopathic diabetes, drug- or chemical-induced diabetes, Stiff-man syndrome, lipoatrophic diabetes, latent autoimmune diabetes in adults (LADA).

Obesity includes overweight, central/upper body obesity, peripheral/lower body obesity, morbid obesity, osteosarcopenic obesity syndrome (OSO), pediatric obesity, Mendelian (monogenic) syndromic obesity, Mendelian non-syndromic obesity, polygenic obesity. Metabolic disorders, diabetes, obesity and the type of subject treated have been earlier defined herein.

Liver inflammation and/or fibrosis includes autoimmune hepatitis, viral hepatitis including hepatitis A, B, C, D and E, alcoholic hepatitis, non-alcoholic steatohepatitis (NASH) and liver cirrhosis.

Cancer includes astrocytoma, glioma, leukemia, lymphoma, melanoma, myeloma, neuroblastoma, sarcoma (including chondrosarcoma, fibrosarcoma, rhabdomyosarcoma, and osteosarcoma), schwannoma, seminoma, and carcinomas of the bladder, breast, cervix, colon, endometrium, esophagus, gallbladder, kidney, liver, lung, ovary, prostate, pancreas, rectum, skin, stomach and thyroid. A preferred cancer is liver cancer, preferably hepatocellular carcinoma. In one embodiment said method or use is performed in vitro, for instance using a cell culture. Preferably, said method or use is in vivo. Each feature of these methods/uses has already been defined herein.

In a method of the invention, a viral expression construct and/or a vector and/or a nucleic acid molecule and/or a composition may be combined with an additional compound known to be used for treating metabolic disorders, preferably diabetes and/or obesity in an individual.

In another method of the invention, a viral expression construct and/or a vector and/or a nucleic acid molecule and/or a composition may be combined with an additional compound known to be used for treating liver inflammation and/or fibrosis. In yet another method of the invention, a viral expression construct and/or a vector and/or a nucleic acid molecule and/or a composition may be combined with an additional compound known to be used for extending healthy lifespan.

In yet another method of the invention, a viral expression construct and/or a vector and/or a nucleic acid molecule and/or a composition may be combined with an additional compound known to be used for treating cancer, preferably liver cancer.

In a preferred embodiment, a treatment in a use or in a method according to the invention does not have to be repeated. Alternatively in a use or a method according to the invention said administration of the viral expression construct or of said composition may be repeated each year or each 2, 3, 4, 5, 6 years.

### General Definitions

### Identity/Similarity

In the context of the invention, a protein fragment or a polypeptide or a peptide or a derived peptide as Fibroblast growth factor 21 (FGF21) is represented by an amino acid sequence.

In the context of the invention, a nucleic acid molecule as a nucleic acid molecule encoding a FGF21 is represented by a nucleic acid or nucleotide sequence which encodes a protein fragment or a polypeptide or a peptide or a derived peptide. A nucleic acid molecule may comprise a regulatory region.

It is to be understood that each nucleic acid molecule or protein fragment or polypeptide or peptide or derived peptide or construct as identified herein by a given Sequence Identity Number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide. Throughout this application, each time one refers to a specific nucleotide sequence SEQ ID NO (take SEQ ID NO: X as example) encoding a given protein fragment or polypeptide or peptide or derived peptide, one may replace it by:
i. a nucleotide sequence comprising a nucleotide sequence that has at least 60% sequence identity or similarity with SEQ ID NO: X;
ii. a nucleotide sequence the sequence of which differs from the sequence of a nucleic acid molecule of (i) due to the degeneracy of the genetic code; or,
iii. a nucleotide sequence that encodes an amino acid sequence that has at least 60% amino acid identity or similarity with an amino acid sequence encoded by a nucleotide sequence SEQ ID NO: X.

Throughout this application, each time one refers to a specific amino acid sequence SEQ ID NO (take SEQ ID NO: Y as example), one may replace it by: a polypeptide comprising an amino acid sequence that has at least 60% sequence identity or similarity with amino acid sequence SEQ ID NO: Y.

Each nucleotide sequence or amino acid sequence described herein by virtue of its identity or similarity percentage (at least 60%) with a given nucleotide sequence or amino acid sequence respectively has in a further preferred embodiment an identity or a similarity of at least 65%, 70%, 75%, 80%, 85%, 90%, 95%, 97%, 98%, 99% or more identity or similarity with the given nucleotide or amino acid sequence respectively. In a preferred embodiment, sequence identity or similarity is determined by comparing the whole length of the sequences as identified herein. Unless otherwise indicated herein, identity or similarity with a given SEQ ID NO means identity or similarity based on the full length of said sequence (i.e. over its whole length or as a whole).

Each non-coding nucleotide sequence (i.e. of a promoter or of another regulatory region) could be replaced by a nucleotide sequence comprising a nucleotide sequence that has at least 60% sequence identity or similarity with a specific nucleotide sequence SEQ ID NO (take SEQ ID NO: A as example). A preferred nucleotide sequence has at least 60%, 65%, 70%, 75%, 80%, 85%, 90%, 95%, 97%, 98%, 99%, 100% identity with SEQ ID NO: A. Identity may be assessed over the whole SEQ ID NO or over part thereof as explained herein. In a preferred embodiment, such non-coding nucleotide sequence such as a promoter exhibits or exerts at least an activity of such a non-coding nucleotide sequence such as an activity of a promoter as known to the skilled person.

“Sequence identity” is herein defined as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO or on part thereof. Part thereof preferably means at least 10%, 20%, 30%, 40%, 50%, 60%, 70%, 80%, 90%, or 100% of both SEQ ID NO. In the art, “identity” also means the degree of sequence relatedness between amino acid or nucleic acid sequences, as the case may be, as determined by the match between strings of such sequences.

“Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, MD 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, WI. The aforementioned parameters are the default parameters for amino acid comparisons (along with no penalty for end gaps). Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

### Gene or Coding Sequence

“Gene” or “coding sequence” or “nucleic acid” or “nucleotide sequence” or “nucleic” refers to a DNA or RNA region (the transcribed region) which “encodes” a particular protein such as a FGF21. A coding sequence is transcribed (DNA) and translated (RNA) into a polypeptide when placed under the control of an appropriate regulatory region, such as a promoter. A gene may comprise several operably linked fragments, such as a promoter, a 5′ leader sequence, an intron, a coding sequence and a 3′ nontranslated sequence or 3′ untranslated region (3′ UTR), comprising a polyadenylation site or a signal sequence. A chimeric or recombinant gene (such as a FGF21 gene) is a gene not normally found in nature, such as a gene in which for example the promoter is not associated in nature with part or all of the transcribed DNA region. “Expression of a gene” refers to the process wherein a gene is transcribed into an RNA and/or translated into an active protein.

### Promoter

As used herein, the term “promoter” refers to a nucleic acid fragment that functions to control the transcription of one or more genes (or coding sequence), located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “organ-specific” or “tissue-specific” promoter is a promoter that is active in a specific type of organ or tissue, respectively. Organ-specific and tissue-specific promoters regulate expression of one or more genes (or coding sequence) primarily in one organ or tissue, but can allow detectable level (“leaky”) expression in other organs or tissues as well. Leaky expression in other organs or tissues means at least one-fold, at least two-fold, at least three-fold, at least four-fold or at least five-fold lower, but still detectable expression as compared to the organ-specific or tissue-specific expression, as evaluated by standard assays known to the skilled person (e.g. PCR, Western blot analysis, ELISA). The maximum number of organs or tissues where leaky expression may be detected is five, six, seven or eight. An “adipose tissue-specific promoter” is a promoter that is capable of initiating transcription in the adipose tissue, whilst still allowing for any leaky expression in other (maximum five, six, seven or eight) organs and parts of the body. Transcription in the adipose tissue can be detected in adipose tissue and adipose cells, such as white adipocytes, brown adipocytes, beige adipocytes, preadipocytes, stromal vascular cells. A “liver-specific promoter” is a promoter that is capable of initiating transcription in the liver, whilst still allowing for any leaky expression in other (maximum five, six, seven or eight) organs and parts of the body. Transcription in the liver can be detected in liver tissue and liver cells, such as hepatocytes, Kupffer cells and/or oval cells. Similarly, a “skeletal muscle promoter” is a promoter that is capable of initiating transcription in skeletal muscle, whilst still allowing for any leaky expression in other (maximum five, six, seven or eight) organs and parts of the body. Transcription in the skeletal muscle can be detected in skeletal muscle cells, such as myocytes, myoblasts, satellite cells.

A “ubiquitous promoter” is active in substantially all tissues, organs and cells of an organism.

Suitable promoters for organ-specific and/or tissue-specific expression of a nucleotide sequence encoding a FGF21 include the human al-antitrypsin promoter, the al-antitrypsin promoter in combination with the hepatocyte control region (HCR) enhancer from the apolipoprotein E, the albumin promoter, the major urinary protein promoter, the phosphoenolpyruvate carboxykinase (PEPCK) promoter, the liver-enriched protein activator promoter, the transthyretin promoter, the thyroxine binding globulin promoter, the apolipoprotein Al promoter, the liver fatty acid binding protein promoter, the phenylalanine hydroxylase promoter, the adipocyte protein 2 (aP2, also known as fatty acid binding protein 4 (FABP4)) promoter, the PPARy promoter, the adiponectin promoter, the phosphoenolpyruvate carboxykinase (PEPCK) promoter, the promoter derived from human aromatase cytochrome p450 (p450arom) the mini/aP2 promoter (composed of the adipose-specific aP2 enhancer and the basal aP2 promoter), the uncoupling protein 1 (UCP1) promoter, the mini/UCPI promoter (composed of the adipose-specific UCP1 enhancer and the basal UCPI promoter), the adipsin promoter, the leptin promoter, the Foxa-2 promoter, the myosin light-chain promoter, the myosin heavy-chain promoter, the desmin promoter, the C5-12 promoter, the muscle creatine kinase (MCK) promoter, the smooth muscle alpha-actin promoter, the CK6 promoter, the Unc-45 Myosin Chaperone B promoter, the basal MCK promoter in combination with copies of the MCK enhancer, the Enh358MCK promoter (combination of the MCK enhancer with the 358 bp proximal promoter of the MCK gene).

### Operably Linked

“Operably linked” is defined herein as a configuration in which a control sequence such as a promoter sequence or regulating sequence is appropriately placed at a position relative to the nucleotide sequence of interest, preferably coding for a FGF21 such that the promoter or control or regulating sequence directs or affects the transcription and/or production or expression of the nucleotide sequence of interest, preferably encoding a FGF21 in a cell and/or in a subject. For instance, a promoter is operably linked to a coding sequence if the promoter is able to initiate or regulate the transcription or expression of a coding sequence, in which case the coding sequence should be understood as being “under the control of′ the promoter. When one or more nucleotide sequences and/or elements comprised within a construct are defined herein to be “configured to be operably linked to an optional nucleotide sequence of interest”, said nucleotide sequences and/or elements are understood to be configured within said construct in such a way that these nucleotide sequences and/or elements are all operably linked to said nucleotide sequence of interest once said nucleotide sequence of interest is present in said construct.

### Viral Expression Construct

An expression construct carries a genome that is able to stabilize and remain episomal in a cell. Within the context of the invention, a cell may mean to encompass a cell used to make the construct or a cell wherein the construct will be administered. Alternatively a construct is capable of integrating into a cell's genome, e.g. through homologous recombination or otherwise. A particularly preferred expression construct is one wherein a nucleotide sequence encoding a FGF21 as defined herein is operably linked to a promoter as defined herein wherein said promoter is capable of directing expression of said nucleotide sequence (i.e. coding sequence) in a cell. Preferably, said promoter directs expression of said nucleotide sequence in at least one cell of a specific organ and/or a specific tissue. Preferably, said promoter directs expression of said nucleotide sequence in at least one cell of liver, adipose tissue and/or skeletal muscle. Preferably, said promoter directs expression in at least 10%, 20%, 30%, 40%, 40%, 60%, 70%, 80%, 90%, or 100% of cells of liver, adipose tissue and/or skeletal muscle. In the context of the invention, a FGF21 to be expressed in the liver, adipose tissue or skeletal muscle refers to the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of FGF21 in the liver, adipose tissue or skeletal muscle as compared to other organs or tissues. Throughout the application, where liver-specific, or adipose-specific or skeletal muscle-specific is mentioned in the context of expression, cell-type specific expression of the cell type(s) making up the liver, the adipose tissue or skeletal muscle is also envisaged, respectively.

The viral expression constructs of the invention comprise a nucleotide sequence in a form “suitable for expression in a mammal”, which means that the viral expression constructs include one or more regulatory sequences, selected on the basis of the mammalian host cells to be used for expression, that is operatively linked to the nucleotide sequence to be expressed. Preferably, said mammalian host cells to be used for expression are human, murine or canine cells.

The viral expression constructs of the invention comprise a nucleotide sequence to be expressed in liver, adipose tissue and/or skeletal muscle.

As used herein, “adipose tissue” refers to tissue composed of mature adipocytes (i.e. fat cells) and a combination of small blood vessels, nerve tissue, lymph nodes and the stromal vascular fraction (SVF). The SVF is composed of endothelial cells, fibroblasts, adipocyte precursor cells (i.e. preadipocytes), and immune cells such as macrophages and T cells. In mammals, two different types of adipose tissues are traditionally distinguished: the white adipose tissue (WAT) and the brown adipose tissue (BAT). In mammals, the adipose tissue is contained in a multi-depot organ. Adipose depots include but are not limited to epidydymal WAT (eWAT), inguinal WAT (iWAT), retroperitoneal WAT (rWAT), mesenteric WAT (mWAT), interscapular BAT (iBAT).

As used herein, “skeletal muscle” refers to the tissue composed of muscle fibers. A muscle fiber, also known as a myofiber, is a single multinucleated or syncitial cell that results from the fusion of many hundreds of myoblasts, some of which remain in the mature muscle as undifferentiated cells known as satellite cells. Individual muscle fibers are surrounded by a connective tissue called endomysium. Around 10 to 100 muscle fibers form fascicles, or bundles, which are themselves surrounded by another connective tissue layer called the perimysium. Finally, the skeletal muscle is formed by groups of fascicles that are surrounded also by another connective tissue layer called the epimysium. In addition to muscle fibers, skeletal muscle is also composed of numerous blood vessels and nerves. The ends of muscles converge in dense connective tissue structures, the tendons and aponeuroses that mediate attachment of muscles to the periosteum of bones or to the connective tissue of other muscles.

As used herein, “liver” refers to the tissue composed of hepatocytes. Hepatocytes represent about 50-70% of the cells in the liver. In addition to hepatocytes, the liver is composed of endothelial cells, perisinusoidal cells, oval cells, Kupffer cells and stellate cells (Ito cells). When activated by Kupffer cells, the stellate cells transform into myofibroblasts. Central veins and portal tracks (portal triads) that contain preterminal branches of the hepatic artery, the hepatic portal vein, bile ductules and lymphatic vessels are also found in the liver.

Such a preferred expression construct is said to comprise an expression cassette. An expression cassette as used herein comprises or consists of a nucleotide sequence encoding a FGF21, being operably linked to a promoter wherein said promoter is capable of directing expression of said nucleotide sequence. In an embodiment, an expression cassette as used herein comprises or consists of a nucleotide sequence encoding a FGF21, a promoter and at least one nucleotide sequence encoding a target sequence of a microRNA expressed in the liver and at least one nucleotide sequence encoding a target sequence of a microRNA expressed in the heart. In one embodiment the described expression cassettes contain nucleotide sequences encoding target sequences for a microRNA expressed in the liver and/or a microRNA expressed in the heart with perfect complementarity to their cognate microRNAs. In another embodiment, the described expression cassettes contain one or more nucleotide sequence(s) encoding microRNA binding sites with imperfect complementarity (one mismatch/five consecutive nucleotides). In yet another embodiment, the expression cassettes may contain both nucleotide sequences encoding perfect and imperfect microRNA binding sites. Expression cassettes can therefore be tailored to result in varying levels of regulation by using nucleotide sequences encoding single perfect, multiple perfect, single imperfect, multiple imperfect or a combination of perfect and imperfect target sites for microRNAs. Further, nucleotide sequence encoding target sites for different microRNAs may be used, therefore permitting a gene to be regulated by multiple microRNAs. A preferred location for the nucleotide sequence encoding a target sequence of a microRNA is the 3′UTR. However, nucleotide sequences (encoding target sequences) inserted into either a coding sequence or 5′UTR sequences may also be used.

The choice of nucleotide sequence encoding a target sequence of a microRNA is determined by the desired expression pattern. The presence of an endogenous microRNA in a cell will inhibit expression of a gene or coding sequence from an expression construct which contains a nucleotide sequence encoding a target sequence for said microRNA. For expression of the gene or coding sequence of interest to be inhibited in a given cell-type, a nucleotide sequence encoding a target sequence that is recognized by a microRNA present in that cell-type is chosen.

A viral expression construct is an expression construct which is intended to be used in gene therapy. It is designed to comprise part of a viral genome as later defined herein. Expression constructs disclosed herein could be prepared using recombinant techniques in which a nucleotide sequence encoding said FGF21 is expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34:315 (describing cassette mutagenesis). Typically, a nucleic acid or nucleotide sequence encoding a FGF21 is used in an expression construct or expression vector. The phrase “expression vector” or “vector” generally refers to a nucleotide sequence that is capable of effecting expression of a gene or a coding sequence in a host compatible with such sequences. These expression vectors typically include at least suitable promoter sequences and optionally, transcription termination signals. An additional factor necessary or helpful in effecting expression can also be used as described herein. A nucleic acid or DNA or nucleotide sequence encoding a FGF21 is incorporated into a DNA construct capable of introduction into and expression in an in vitro cell culture. Specifically, a DNA construct is suitable for replication in a prokaryotic host, such as bacteria, e.g., E. coli, or can be introduced into a cultured mammalian, plant, insect, (e.g., Sf9), yeast, fungi or other eukaryotic cell lines. A DNA construct prepared for introduction into a particular host may include a replication system recognized by the host, an intended DNA segment encoding a desired polypeptide, and transcriptional and translational initiation and termination regulatory sequences operably linked to the polypeptide-encoding segment. The term “operably linked” has already been defined herein. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis.

The selection of an appropriate promoter sequence generally depends upon the host cell selected for the expression of a DNA segment. Examples of suitable promoter sequences include prokaryotic, and eukaryotic promoters well known in the art (see, e.g. Sambrook and Russell, 2001, supra). A transcriptional regulatory sequence typically includes a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). An expression vector includes the replication system and transcriptional and translational regulatory sequences together with the insertion site for the polypeptide encoding segment. In most cases, the replication system is only functional in the cell that is used to make the vector (bacterial cell as E. Coli). Most plasmids and vectors do not replicate in the cells infected with the vector. Examples of workable combinations of cell lines and expression vectors are described in Sambrook and Russell (2001, supra) and in Metzger et al. (1988) Nature 334:31-36. For example, suitable expression vectors can be expressed in, yeast, e.g. S.cerevisiae, e.g., insect cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media.

Alternatively, a host cell is a cell that is part of a multicellular organism such as a transgenic plant or animal.

### Viral Vector

A viral vector or a viral gene therapy vector is a vector that comprises a viral expression construct as defined above.

A viral vector or a viral gene therapy vector is a vector that is suitable for gene therapy. Vectors that are suitable for gene therapy are described in Anderson 1998, Nature 392:25-30; Walther and Stein, 2000, Drugs 60:249-71; Kay et al., 2001, Nat. Med. 7:33-40; Russell, 2000, J. Gen. Virol. 81:2573-604; Amado and Chen, 1999, Science 285:674-6; Federico, 1999, Curr. Opin. Biotechnol.10:448-53; Vigna and Naldini, 2000, J. Gene Med. 2:308-16; Marin et al., 1997, Mol. Med. Today 3:396-403; Peng and Russell, 1999, Curr. Opin. Biotechnol. 10:454-7; Sommerfelt, 1999, J. Gen. Virol. 80:3049-64; Reiser, 2000, Gene Ther. 7:910-3; and references cited therein.

A particularly suitable gene therapy vector includes an adenoviral and adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications, (Russell, 2000, J. Gen. Virol. 81:2573-2604; Goncalves, 2005, Virol J. 2 (1): 43) as indicated above. AAV vectors are even more preferred since they are known to result in very stable long term expression of transgene expression (up to 9 years in dog (Niemeyer et al, Blood. 2009 Jan. 22; 113 (4): 797-806) and ˜10 years in human (Buchlis, G. et al., Blood. 2012 Mar. 29; 119 (13): 3038-41). Preferred adenoviral vectors are modified to reduce the host response as reviewed by Russell (2000, supra). Method for gene therapy using AAV vectors are described by Wang et al., 2005, J Gene Med. March 9 (Epub ahead of print), Mandel et al., 2004, Curr Opin Mol Ther. 6 (5): 482-90, and Martin et al., 2004, Eye 18 (11): 1049-55, Nathwani et al, N Engl J Med. 2011 Dec. 22; 365 (25): 2357-65, Apparailly et al, Hum Gene Ther. 2005 April; 16 (4): 426-34.

Another suitable gene therapy vector includes a retroviral vector. A preferred retroviral vector for application in the present invention is a lentiviral based expression construct. Lentiviral vectors have the ability to infect and to stably integrate into the genome of dividing and non-dividing cells (Amado and Chen, 1999 Science 285:674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10:448-53) and Vigna et al. (2000, J Gene Med 2000; 2:308-16).

Other suitable gene therapy vectors include an adenovirus vector, a herpes virus vector, a polyoma virus vector or a vaccinia virus vector.

A gene therapy vector comprises a nucleotide sequence encoding a FGF21 to be expressed, whereby said nucleotide sequence is operably linked to the appropriate regulatory sequences. Such regulatory sequence will at least comprise a promoter sequence. Suitable promoters for expression of a nucleotide sequence encoding a FGF21 from a gene therapy vector include e.g. CMV promoter, viral long terminal repeat promoters (LTRs), such as those from murine moloney leukaemia virus (MMLV) rous sarcoma virus, or HTLV-1, the simian virus 40 (SV 40) early promoter, the CAG promoter, the al-antitrypsin promoter, the mini/aP2 promoter, the mini/UCP1 promoter, the C5-12 promoter and the herpes simplex virus thymidine kinase promoter. Several inducible promoter systems have been described that may be induced by the administration of small organic or inorganic compounds. Such inducible promoters include those controlled by heavy metals, such as the metallothionine promoter (Brinster et al. 1982 Nature 296:39-42; Mayo et al. 1982 Cell 29:99-108), RU-486 (a progesterone antagonist) (Wang et al. 1994 Proc. Natl. Acad. Sci. USA 91:8180-8184), steroids (Mader and White, 1993 Proc. Natl. Acad. Sci. USA 90:5603-5607), tetracycline (Gossen and Bujard 1992 Proc. Natl. Acad. Sci. USA 89:5547-5551; U.S. Pat. No. 5,464,758; Furth et al. 1994 Proc. Natl. Acad. Sci. USA 91:9302-9306; Howe et al. 1995 J. Biol. Chem. 270:14168-14174; Resnitzky et al. 1994 Mol. Cell. Biol. 14: 1669-1679; Shockett et al. 1995 Proc. Natl. Acad. Sci. USA 92:6522-6526) and the tTAER system that is based on the multi-chimeric transactivator composed of a tetR polypeptide, as activation domain of VP16, and a ligand binding domain of an estrogen receptor (Yee et al., 2002, U.S. Pat. No. 6,432,705).

A gene therapy vector may optionally comprise a further nucleotide sequence coding for a further polypeptide.

A gene therapy vector is preferably formulated in a composition or pharmaceutical composition as defined herein. In this context, a composition or pharmaceutical composition may comprise a suitable pharmaceutical carrier as earlier defined herein.

### Adeno-Associated Virus Vector (AAV Vector)

A preferred viral vector or a preferred gene therapy vector is an AAV vector. An AAV vector as used herein preferably comprises a recombinant AAV vector (rAAV vector). A “rAAV vector” as used herein refers to a recombinant vector comprising part of an AAV genome encapsidated in a protein shell of capsid protein derived from an AAV serotype as explained herein. Part of an AAV genome may contain the inverted terminal repeats (ITR) derived from an adeno-associated virus serotype, such as AAV1, AAV2, AAV3, AAV4, AAV5 and others. Preferred ITRs are those of AAV2 which are represented by sequences comprising or consisting of SEQ ID NO: 48 (5′ ITR) and SEQ ID NO: 49 (3′ ITR). The invention also preferably encompasses the use of a sequence having at least 80% (or at least 81%, 82%, 83%, 84%, 85%, 86%, 87%, 87%, 88%, 89%, 90%, 91%, 92%, 93%, 94%, 95%, 96%, 97%, 98%, 99% or 100%) identity with SEQ ID NO: 48 as 5′ITR and a sequence having at least 80% identity with SEQ ID NO: 49 as 3′ITR.

Protein shell comprised of capsid protein may be derived from an AAV serotype such as AAV1, 2, 3, 4, 5 and others. A preferred AAV capsid is an AAV1, AAV3, AAV8, AAV9 capsid. A preferred ITR is from the AAV2. A protein shell may also be named a capsid protein shell. rAAV vector may have one or preferably all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, 85%, 90%, 95%, 97%, 98%, 99% or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the present invention a capsid protein shell may be of a different serotype than the rAAV vector genome ITR.

A nucleic acid molecule represented by a nucleic acid sequence of choice is preferably inserted between the rAAV genome or ITR sequences as identified above, for example an expression construct comprising an expression regulatory element operably linked to a coding sequence and a 3′ termination sequence. Said nucleic acid molecule may also be called a transgene.

“AAV helper functions” generally refers to the corresponding AAV functions required for rAAV replication and packaging supplied to the rAAV vector in trans. AAV helper functions complement the AAV functions which are missing in the rAAV vector, but they lack AAV ITRs (which are provided by the rAAV vector genome). AAV helper functions include the two major ORFs of AAV, namely the rep coding region and the cap coding region or functional substantially identical sequences thereof. Rep and Cap regions are well known in the art, see e.g. Chiorini et al. (1999, J. of Virology, Vol 73 (2): 1309-1319) or U.S. Pat. No. 5,139,941, incorporated herein by reference. The AAV helper functions can be supplied on an AAV helper construct. Introduction of the helper construct into the host cell can occur e.g. by transformation, transfection, or transduction prior to or concurrently with the introduction of the rAAV genome present in the rAAV vector as identified herein. The AAV helper constructs of the invention may thus be chosen such that they produce the desired combination of serotypes for the rAAV vector's capsid protein shell on the one hand and for the rAAV genome present in said rAAV vector replication and packaging on the other hand.

“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via plasmids, as described in U.S. Pat. No. 6,531,456 incorporated herein by reference.

A “transgene” is herein defined as a gene or a coding sequence or a nucleic acid molecule (i.e. a molecule encoding a FGF21) that has been newly introduced into a cell, i.e. a gene that may be present but may normally not be expressed or expressed at an insufficient level in a cell. In this context, “insufficient” means that although said FGF21 is expressed in a cell, a condition and/or disease as defined herein could still be developed. In this case, the invention allows the over-expression of a FGF21. The transgene may comprise sequences that are native to the cell, sequences that naturally do not occur in the cell and it may comprise combinations of both. A transgene may contain sequences coding for a FGF21 and/or additional proteins as earlier identified herein that may be operably linked to appropriate regulatory sequences for expression of the sequences coding for a FGF21 in the cell. Preferably, the transgene is not integrated into the host cell's genome.

“Transduction” refers to the delivery of a FGF21 into a recipient host cell by a viral vector. For example, transduction of a target cell by a rAAV vector of the invention leads to transfer of the rAAV genome contained in that vector into the transduced cell. “Host cell” or “target cell” refers to the cell into which the DNA delivery takes place, such as the muscle cells of a subject. AAV vectors are able to transduce both dividing and non-dividing cells.

### Production of an AAV Vector

The production of recombinant AAV (rAAV) for vectorizing transgenes have been described previously. See Ayuso E, et al., Curr. Gene Ther. 2010; 10:423-436, Okada T, et al., Hum. Gene Ther. 2009; 20:1013-1021, Zhang H, et al., Hum. Gene Ther. 2009; 20:922-929, and Virag T, et al., Hum. Gene Ther. 2009; 20:807-817. These protocols can be used or adapted to generate the AAV of the invention. In one embodiment, the producer cell line is transfected transiently with the polynucleotide of the invention (comprising the expression cassette flanked by ITRs) and with construct(s) that encodes rep and cap proteins and provides helper functions. In another embodiment, the cell line supplies stably the helper functions and is transfected transiently with the polynucleotide of the invention (comprising the expression cassette flanked by ITRs) and with construct(s) that encodes rep and cap proteins. In another embodiment, the cell line supplies stably the rep and cap proteins and the helper functions and is transiently transfected with the polynucleotide of the invention. In another embodiment, the cell line supplies stably the rep and cap proteins and is transfected transiently with the polynucleotide of the invention and a polynucleotide encoding the helper functions. In yet another embodiment, the cell line supplies stably the polynucleotide of the invention, the rep and cap proteins and the helper functions. Methods of making and using these and other AAV production systems have been described in the art. See Muzyczka N, et al., U.S. Pat. No. 5,139,941, Zhou X, et al., U.S. Pat. No. 5,741,683, Samulski R, et al., U.S. Pat. No. 6,057,152, Samulski R, et al., U.S. Pat. No. 6,204,059, Samulski R, et al., U.S. Pat. No. 6,268,213, Rabinowitz J, et al., U.S. Pat. No. 6,491,907, Zolotukhin S, et al., U.S. Pat. No. 6,660,514, Shenk T, et al., U.S. Pat. No. 6,951,753, Snyder R, et al., U.S. Pat. No. 7,094,604, Rabinowitz J, et al., U.S. Pat. No. 7,172,893, Monahan P, et al., U.S. Pat. No. 7,201,898, Samulski R, et al., U.S. Pat. No. 7,229,823, and Ferrari F, et al., U.S. Pat. No. 7,439,065.

The rAAV genome present in a rAAV vector comprises at least the nucleotide sequences of the inverted terminal repeat regions (ITRs) of one of the AAV serotypes (preferably the ones of serotype AAV2 as disclosed earlier herein), or nucleotide sequences substantially identical thereto or nucleotide sequences having at least 60% identity thereto, and nucleotide sequence encoding a FGF21 (under control of a suitable regulatory element) inserted between the two ITRs. A vector genome requires the use of flanking 5′ and a 3′ ITR sequences to allow for efficient packaging of the vector genome into the rAAV capsid.

The complete genome of several AAV serotypes and corresponding ITR has been sequenced (Chiorini et al. 1999, J. of Virology Vol. 73, No. 2, p1309-1319). They can be either cloned or made by chemical synthesis as known in the art, using for example an oligonucleotide synthesizer as supplied e.g. by Applied Biosystems Inc. (Fosters, CA, USA) or by standard molecular biology techniques. The ITRs can be cloned from the AAV viral genome or excised from a vector comprising the AAV ITRs. The ITR nucleotide sequences can be either ligated at either end to the nucleotide sequence encoding one or more therapeutic proteins using standard molecular biology techniques, or the AAV sequence between the ITRs can be replaced with the desired nucleotide sequence.

Preferably, the rAAV genome as present in a rAAV vector does not comprise any nucleotide sequences encoding viral proteins, such as the rep (replication) or cap (capsid) genes of AAV. This rAAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

The rAAV genome as present in said rAAV vector further comprises a promoter sequence operably linked to the nucleotide sequence encoding a FGF21. Preferred promoter sequences are promoters which confer expression in skeletal muscle cells and/or skeletal muscle, in liver cells and/or liver and in adipose cells and/or adipose tissue. Examples of such promoters include a CMV, a CAG, a mini/aP2, a mini/UCP1, a C5-12 and a hAAT promoter as earlier defined herein.

A suitable 3′ untranslated sequence may also be operably linked to the nucleotide sequence encoding a FGF21. Suitable 3′ untranslated regions may be those naturally associated with the nucleotide sequence or may be derived from different genes, such as for example the SV40 polyadenylation signal (SEQ ID NO: 50) and the rabbit β-globin polyadenylation signal (SEQ ID NO: 51).

Optionally, additional nucleotide sequences may be operably linked to the nucleotide sequence(s) encoding a FGF21, such as nucleotide sequences encoding signal sequences, nuclear localization signals, expression enhancers, and the like.

### Codon Optimization

“Codon optimization”, as used herein, refers to the processes employed to modify an existing coding sequence, or to design a coding sequence, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. For example, to suit the codon preference of mammalians, preferably of murine, canine or human expression hosts. Codon optimization also eliminates elements that potentially impact negatively RNA stability and/or translation (e. g. termination sequences, TATA boxes, splice sites, ribosomal entry sites, repetitive and/or GC rich sequences and RNA secondary structures or instability motifs).

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a viral expression construct, viral vector, composition, gene therapy composition, as defined herein may comprise additional component(s) than the ones specifically identified, said additional component(s) not altering the unique characteristic of the invention.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “approximately” or “about” when used in association with a numerical value (approximately 10, about 10) preferably means that the value may be the given value of 10 more or less 1% of the value.

All patent and literature references cited in the present specification are hereby incorporated by reference in their entirety. Each embodiment as identified herein may be combined together unless otherwise indicated.

The invention is further explained in the following examples. These examples do not limit the scope of the invention, but merely serve to clarify the invention.

### FIGURE LEGENDS

FIGS. 1A-1H. Prevention of obesity by intra-eWAT administration of AAV9-CAG-moFGF21-dmiRT vectors in C57B16 mice. (FIG. 1A) Schematic representation of the AAV-CAG-moFGF21-doublemiRT vectors. The expression cassette contained the CAG promoter, a murine codon-optimized FGF21 coding sequence and four tandem repeats of the miRT122a sequence and four tandems repeats of the miRT1 sequence cloned in the 3′ untranslated region of the expression cassette. ITRs from AAV2 flanked the expression cassette. The schematic representation is not to scale. CAG: chicken β-actin promoter/CMV enhancer; pA: polyA. (FIG. 1B) Expression levels of FGF21 in metabolic tissues. The expression levels of the murine codon-optimized FGF21 coding sequence were measured by RTqPCR in eWAT, iWAT, iBAT and liver of C57B16 mice, and normalized with Rplp0 values (n=8-11 animals/group). (FIG. 1C) Circulating levels of FGF21 (n=8-11 animals/group). (FIGS. 1D-1E) Expression levels of FGF21R1 (FIG. 1D) and β-Klotho (FIG. 1E) in metabolic tissues. The expression levels of the FGF21 receptor 1 (FGF21R1) and β-Klotho were measured by RTqPCR in eWAT, iWAT, iBAT and liver of C57B16 mice, and normalized with Rplp0 values (n=7 animals/group). (FIG. 1F) Body weight evolution. Body weight was measured weekly (n=8-11 animals/group). (FIG. 1G) Representative image of animals. (FIG. 1H) Weight of tissues. Weight of eWAT, iWAT, rWAT, mWAT, iBAT and liver of chow- and HFD-fed C57B16 mice treated intra-eWAT with AAV vectors (n=8-11 animals/group). Analyses were performed 14 weeks after intra-eWAT administration of 10¹² vg of AAV9-CAG-moFGF21-doublemiRT or AAV9-CAG-null vectors. Results are expressed as the mean±SEM. ND, not detected. HFD, high fat diet. AU, arbitrary units, eWAT, epididymal white adipose tissue, iWAT, inguinal white adipose tissue, rWAT, retroperitoneal white adipose tissue, mWAT, mesenteric white adipose tissue. iBAT interscapular brown adipose tissue. * p<0.05 vs AAV9-CAG-null chow, ** p<0.01 vs AAV9-CAG-null chow, *** p<0.001 vs AAV9-CAG-null chow, $ p<0.05 vs AAV9-CAG-null HFD, $$ p<0.01 vs AAV9-CAG-null HFD, $$$ p<0.001 vs AAV9-CAG-null HFD.

FIGS. 2A-2C. Histological analysis of adipose tissue and liver of C57B16 mice treated intra-eWAT with AAV9-CAG-moFGF21-doublemiRT vectors. (FIG. 2A) Representative images of sections stained with hematoxylin and eosin of epididymal white adipose tissue (eWAT), inguinal white adipose tissue (iWAT) interscapular brown adipose tissue (iBAT) and liver of chow- and HFD-fed C57B16 mice treated intra-eWAT with AAV9-CAG-moFGF21-doublemiRT or AAV9-CAG-null vectors. Original magnification X100. (FIG. 2B) Mean area of white adipocytes of eWAT (n=4 animals/group). (FIG. 2C) Frequency distribution of area of white adipocytes of eWAT (n=4 animals/group). Analyses were performed 14 weeks after intra-eWAT administration of 10¹² vg of AAV9-CAG-moFGF21-doublemiRT or AAV9-CAG-null vectors. Results are expressed as the mean±SEM. HFD, high fat diet. ** p<0.01 vs AAV9-CAG-null chow, *** p<0.001 vs AAV9-CAG-null chow, $$ p<0.01 vs AAV9-CAG-null HFD, $$$ p<0.001 vs AAV9-CAG-null HFD.

FIGS. 3A-3H. Increased energy expenditure and insulin sensitivity in C57B16 mice treated intra-eWAT with AAV9-CAG-moFGF21-doublemiRT vectors. (FIGS. 3A-3B) Expression levels of UCP1 (FIG. 3A) and Dio2 (FIG. 3B). The expression levels of UCP1 and Dio2 were measured by RTqPCR in iWAT and normalized with Rplp0 values (n=7 animals/group). (FIG. 3C) Energy metabolism. The energy expenditure (EE) was measured with indirect open circuit calorimeter. Oxygen consumption and carbon dioxide production were monitored simultaneously. Data were taken 9 weeks post-AAV administration during the light cycle (basal state) and dark cycle (activity phase) and adjusted for body weight (n=8-11 animals/group). (FIG. 3D) Liver triglyceride content (n=8-10 animals/group). (FIGS. 3E-3F) Serum triglyceride (FIG. 3E) and cholesterol (FIG. 3F) levels (n=8-11 animals/group). (FIG. 3G) Intraperitoneal insulin tolerance test. Mice were given an intraperitoneal injection of 0.75 U insulin/kg body weight and blood glucose levels were measured at the indicated time points (n=6-11 animals/group). The test was performed 11 weeks post-AAV administration. (FIG. 3H) Fasted insulin circulating levels. Unless otherwise indicated, analyses were performed 14 weeks after intra-eWAT administration of 10¹² vg of AAV9-CAG-moFGF21-doublemiRT or AAV9-CAG-null vectors. Results are expressed as the mean±SEM. HFD, high fat diet. TG, triglycerides. Chol, cholesterol. * p<0.05 vs AAV9-CAG-null chow, ** p<0.01 vs AAV9-CAG-null chow, *** p<0.001 vs AAV9-CAG-null chow, $ p<0.05 vs AAV9-CAG-null HFD, $$ p<0.01 vs AAV9-CAG-null HFD, $$$ p<0.001 vs AAV9-CAG-null HFD.

FIGS. 4A-4E. Reversion of obesity by intra-eWAT administration of AAV8-CAG-moFGF21-dmiRT vectors in ob/ob mice. (FIG. 4A) Expression levels of FGF21 in metabolic tissues. The expression levels of the murine codon-optimized FGF21 coding sequence were measured by RTqPCR in eWAT, iWAT, iBAT and liver of ob/ob mice, and normalized with Rplp0 values (FIG. 4B) Circulating levels of FGF21. (FIGS. 4C-4D) Body weight (FIG. 4C) and body weight gain (FIG. 4D) evolution. Body weight was measured weekly. (FIG. 4E) Weight of tissues. Weight of eWAT, iWAT, rWAT, mWAT, iBAT and liver of ob/ob mice treated intra-eWAT with AAV vectors. Analyses were performed 16 weeks after intra-eWAT administration of 10¹⁰ vg, 5×10¹⁰ vg, 2×10¹¹ vg or 10¹² vg of AAV8-CAG-moFGF21-doublemiRT or 10¹² vg of AAV8-CAG-null vectors. Results are expressed as the mean±SEM. n=7-8 animals/group. ND, not detected. AU, arbitrary units. eWAT, epididymal white adipose tissue. iWAT, inguinal white adipose tissue. rWAT, retroperitoneal white adipose tissue. mWAT, mesenteric white adipose tissue. iBAT interscapular brown adipose tissue. * p<0.05 vs AAV8-CAG-null, ** p<0.01 vs AAV8-CAG-null, *** p<0.001 vs AAV8-CAG-null.

FIGS. 5A-5B. Improved insulin sensitivity in ob/ob mice treated intra-eWAT with AAV8-CAG-moFGF21-doublemiRT vectors. (FIG. 5A) Intraperitoneal insulin tolerance test. Ob/ob mice were given an intraperitoneal injection of 0.75 U insulin/kg body weight and blood glucose levels were measured at the indicated time points. The test was performed 9 weeks post-AAV administration. (FIG. 5B) Fasted insulin circulating levels 2 months post-AAV. Results are expressed as the mean±SEM, n=7-8 animals/group. * p<0.05 vs AAV8-CAG-null, ** p<0.01 vs AAV8-CAG-null, *** p<0.001 vs AAV8-CAG-null.

FIGS. 6A-61. Reversion of obesity and amelioration of glucose metabolism by intravenous administration of AAV8-hAAT-moFGF21-vectors in ob/ob mice. (FIG. 6A) Schematic representation of AAV-hAAT-moFGF21 vectors. The expression cassette contained the human al-antitrypsin (hAAT) promoter and a murine codon-optimized FGF21 coding sequence. ITRs from AAV2 flanked the expression cassette. The schematic representation is not to scale. pA: polyA. (FIG. 6B) Expression levels of FGF21. The expression levels of the murine codon-optimized FGF21 coding sequence were measured by RTqPCR in the liver of ob/ob mice, and normalized with Rplp0 values. (FIG. 6C) Circulating levels of FGF21. (FIGS. 6D-6E) Body weight (FIG. 6C) and body weight gain (FIG. 6D) evolution. Body weight was measured weekly. (FIG. 6F) Representative image of animals. (FIG. 6G) Weight of tissues. Weight of eWAT, iWAT, rWAT, mWAT, iBAT and liver of ob/ob mice treated intravenously with AAV vectors. (FIG. 6H) Intraperitoneal insulin tolerance test. Ob/ob mice were given an intraperitoneal injection of 0.75 U insulin/kg body weight and blood glucose levels were measured at the indicated time points. The test was performed 9 weeks post-AAV administration. (FIG. 6I) Fasted insulin circulating levels 3 months post-AAV. Unless otherwise indicated, analyses were performed 20 weeks after intravenous administration of 10¹¹ vg or 5×10¹¹ vg of AAV8-hAAT-moFGF21 or 5×10¹¹ vg of AAV8-hAAT-null vectors. Results are expressed as the mean±SEM. n=9-10 animals/group. ND, not detected. AU, arbitrary units. eWAT, epididymal white adipose tissue. iWAT, inguinal white adipose tissue. rWAT, retroperitoneal white adipose tissue. mWAT, mesenteric white adipose tissue. iBAT interscapular brown adipose tissue. * p<0.05 vs AAV8-hAAT-null, ** p<0.01 vs AAV8-hAAT-null, *** p<0.001 vs AAV8-hAAT-null.

FIGS. 7A-7C. Long-term reversion of obesity by intravenous administration of AAV-hAAT-moFGF21 vectors in HFD-fed C57bl6 mice. (FIG. 7A) Circulating levels of FGF21. (FIGS. 7B-7C) Body weight (FIG. 7B) and body weight gain (FIG. 7C) evolution. Body weight was measured weekly. Analyses were performed 52 weeks after intravenous administration of 10¹⁰ vg or 5×10¹⁰ vg of AAV8-hAAT-moFGF21 or 5×10¹⁰ vg of AAV8-hAAT-null vectors. Results are expressed as the mean±SEM, n=9-12 animals/group. *** p<0.001 vs AAV8-hAAT-null chow, $$ p<0.01 vs AAV8-hAAT-null HFD, $$$ p<0.001 vs AAV8-hAAT-null HFD.

FIGS. 8A-8C. Long-term increased energy expenditure and insulin sensitivity by intravenous administration of AAV-hAAT-moFGF21 vectors in HFD-fed C57B16 mice. (FIG. 8A) Energy metabolism. The energy expenditure (EE) was measured with indirect open circuit calorimeter. Oxygen consumption and carbon dioxide production were monitored simultaneously. Data were taken 4 weeks post-AAV administration during the light cycle (basal state) and dark cycle (activity phase) and adjusted for body weight. (FIG. 8B) Intraperitoneal insulin tolerance test. C57B16 mice were given an intraperitoneal injection of 0.75 U insulin/kg body weight and blood glucose levels were measured at the indicated time points. The test was performed 7 weeks post-AAV administration. (FIG. 8C) Fasted and fed insulin circulating levels. Results are expressed as the mean±SEM, n=9-12 animals/group. HFD, high fat diet. * p<0.05 vs AAV8-hAAT-null chow, ** p<0.01 vs AAV8-hAAT-null chow, *** p<0.001 vs AAV8-hAAT-null chow, $ p<0.05 vs AAV8-hAAT-null HFD, $$ p<0.01 vs AAV8-hAAT-null HFD, $$$ p<0.001 vs AAV8-hAAT-null HFD.

FIGS. 9A-9C. Reversion of obesity by intravenous administration of AAV-hAAT-moFGF21 vectors in old HFD-fed mice. (FIG. 9A) Circulating levels of FGF21. (FIGS. 9B-9C) Body weight (FIG. 9B) and body weight gain (FIG. 9C) evolution. Body weight was measured weekly. Analysis were performed 21 weeks after intravenous administration of 10¹⁰ vg, 2×10¹⁰ vg or 5×10¹⁰ vg of AAV8-hAAT-moFGF21 or 5×10¹⁰ vg of AAV8-hAAT-null vectors. Results are expressed as the mean±SEM, n=7-8 animals/group. HFD, high fat diet. *** p<0.05 vs AAV8-hAAT-null chow, $ p<0.05 vs AAV8-hAAT-null HFD, $$ p<0.01 vs AAV8-hAAT-null HFD. $$$ p<0.001 vs AAV8-hAAT-null HFD.

FIGS. 10A-10C. Increased energy expenditure and insulin sensitivity by intravenous administration of AAV-hAAT-moFGF21 vectors in old HFD-fed mice. (FIG. 10A) Energy metabolism. The energy expenditure (EE) was measured with indirect open circuit calorimeter. Oxygen consumption and carbon dioxide production were monitored simultaneously. Data were taken 6 weeks post-AAV administration during the light cycle (basal state) and dark cycle (activity phase) and adjusted for body weight. (FIG. 10B) Intraperitoneal insulin tolerance test. Old C57B16 mice were given an intraperitoneal injection of 0.75 U insulin/kg body weight and blood glucose levels were measured at the indicated time points. The test was performed 9 weeks post-AAV administration. (FIG. 10C) Fasted and fed insulin circulating levels. Results are expressed as the mean±SEM, n=7-8 animals/group. HFD, high fat diet. ** p<0.01 vs AAV8-hAAT-null chow, *** p<0.001 vs AAV8-hAAT-null chow, $ p<0.05 vs AAV8-hAAT-null HFD, $$ p<0.01 vs AAV8-hAAT-null HFD, $$$ p<0.001 vs AAV8-hAAT-null HFD.

FIGS. 11A-11D. Body weight loss by intramuscular administration of AAV-CMV-moFGF21 vectors in C57B16 mice. (FIG. 11A) Schematic representation of the AAV-CMV-moFGF21 vectors. The expression cassette contained the cytomegalovirus (CMV) promoter and a murine codon-optimized FGF21 coding sequence. ITRs from AAV2 flanked the expression cassette. The schematic representation is not to scale. pA: polyA. (FIG. 11B) Circulating FGF21 levels. (FIGS. 11C-11D) Body weight (FIG. 11C) and body weight gain (FIG. 11D) evolution. Body weight was measured weekly. Results are expressed as the mean±SEM. n=6-7 animals/group. * p<0.05 vs AAV1-CMV-null, ** p<0.01 vs AAV1-CMV-null. FGF21 labels in the figure refer to moFGF21 in accordance with this Figure legend.

FIG. 12. Increased FGF21 protein production by codon-optimization of nucleotide sequences encoding human FGF21. Levels of hFGF21 protein in the culture media of HEK293 cells transfected with wild-type hFGF21 or three different versions of codon-optimized human FGF21 sequences. Results are expressed as the mean±SEM. n=3 wells/group. ND, non detected. * p<0.05 vs Non-transfected cells.

FIGS. 13A-13D. Intra-eWAT administration of AAV8-CAG-moFGF21-dmiRT vectors in ob/ob mice.

FIGS. 13A, 13B Representative images of the hematoxylin-eosin staining of (FIG. 13A) eWAT and (FIG. 13B) liver tissue sections obtained from ob/ob animals injected intra-eWAT either null or FGF21-encoding AAV8 vectors at all doses tested. Scale bars: 100 μm for eWAT and 200 μm for liver.

FIG. 13C Glycemia in the fed state.

FIG. 13D Insulinemia in the fed state 3 months post-AAV.

FGF21 labels in the figure refer to moFGF21.

Data information: All values are expressed as mean±SEM. In (FIGS. 13A, 13B) n=6-9 animals/group. In (FIGS. 13C-13D) n=4-8 animals/group. In (I) n=6-8 animals/group. *P<0.05, ** P<0.01 and *** P<0.001 versus the Null-injected group.

FIGS. 14A-14F. Impact of FGF21 gene transfer to the eWAT of ob/ob mice.

FIG. 14A Serum adiponectin levels in 25-week-old ob/ob animals injected intra-eWAT at 11 weeks of age with either AAV8-CAG-null vectors or AAV8-CAG-moFGF21-dmiRT vectors at 4 different doses (1×10¹⁰, 5×10¹⁰, 2×10¹¹, 1×10¹² vg/mouse).

FIG. 14B Quantification by qRT-PCR of the expression of the macrophage marker F4/80 the same groups of animals as in (FIG. 14A).

FIG. 14C Representative images of the immunostaining of eWAT sections from ob/ob mice that received AAV8-CAG-moFGF21-dmiRT vectors for the macrophage-specific marker Mac2. n=4-8/group. Scale bars: 200 μm.

FIG. 14D Weight of the liver in all intra-eWAT treatment groups.

FIGS. 14E, 14F Hepatic triglyceride and cholesterol content in the fed stated in the same cohorts as in (FIG. 14A)

FGF21 labels in the figure refer to moFGF21 in accordance with this Figure legend. Data information: All values are expressed as mean±SEM. In (FIGS. 14A, 14B, 14D) n=4-8 animals/group. * P<0.05, ** P<0.01 and *** P<0.001 versus null-injected ob/ob group.

FIGS. 15A-15E. Reduced obesity and improved insulin sensitivity in ob/ob mice treated with AAV8-hAAT-moFGF21 vectors.

FIG. 15A Representative images of the hematoxylin-eosin staining of eWAT tissue sections obtained from ob/ob animals injected with either null or FGF21-encoding AAV vectors at 1×10¹¹ or 5×10¹¹ vg/mouse.

FIG. 15B Serum adiponectin levels in all groups.

FIG. 15C Representative images of the hematoxylin-eosin staining of liver tissue sections obtained from ob/ob animals injected with either null or FGF21-encoding AAV vectors at 1×10¹¹ or 5×10¹¹ vg/mouse.

FIG. 15D Fed blood glucose levels.

Fed serum Insulin levels 5 months post-AAV. FIG. 15E

FGF21 labels in the figure refer to moFGF21 in accordance with this Figure legend. Data information: All data represent the mean±SEM. In (FIGS. 15A-15C, 15E) n=9-10 animals/group. * P<0.05, ** P<0.01 and *** P<0.001 versus null-injected ob/ob group.

FIGS. 16A-16G. Effects of FGF21 liver gene transfer on ob/ob mice.

FIG. 16A Immunohistochemistry for the macrophage-specific marker Mac2 in eWAT sections from ob/ob mice that received AAV8-hAAT-moFGF21 vectors. Scale bars: 500 μm.

FIGS. 16B, 16C Quantification by qRT-PCR of the expression of the markers of inflammation F4/80 (FIG. 16B) and TNF-α (FIG. 16C) in the same cohorts of mice.

FIGS. 16D, 16E Weight (FIG. 16D) and representative images of the liver (FIG. 16E) obtained from animals belonging to the same experimental groups as in (FIG. 16A).

FIGS. 16F, 16G Hepatic triglyceride and cholesterol content in the fed state in the same cohorts as in (FIG. 16A)

FGF21 labels in the figure refer to moFGF21 in accordance with this Figure legend. Data information: All values are expressed as mean±SEM. In (FIGS. 16B, 16D-16) n=9-10 animals/group. * P<0.05, ** P<0.01 and *** P<0.001 versus null-injected ob/ob group.

FIGS. 17A-17G. AAV8-hAAT-moFGF21 treatment increases the expression of genes involved in glucose uptake and thermogenesis in adipose tissue of ob/ob mice.

FIGS. 17A, 17B Quantification by qRT-PCR of liver PEPCK and G6Pase expression in ob/ob mice injected at 2 months of age with either AAV8-hAAT-null vectors or AAV8-hAAT-moFGF21 vectors.

FIGS. 17C-17F Quantification by qRT-PCR of GLUT1 (FIG. 17C), GLUT4 (FIG. 17D), HKI (FIG. 17E) and HKII (FIG. 17F) expression in eWAT, iWAT and iBAT in the same animals as in (FIG. 17A)

FIG. 17G Relative expression of UCP1 in iBAT in the same cohorts as in (FIG. 17A).

FGF21 labels in the figure refer to moFGF21 in accordance with this Figure legend. Data information: All values are expressed as mean±SEM. In (FIGS. 17A-17G) n=9-10 animals/group. * P<0.05, ** P<0.01 and *** P<0.001 versus null-injected ob/ob group.

FIGS. 18A-18B. AAV8-mediated liver gene transfer of FGF21 counteracts HFD-induced obesity.

FIG. 18A Weight of the epididymal (eWAT), inguinal (iWAT) and retroperitoneal (rWAT) white adipose tissue depots, the liver and the quadriceps obtained from mice treated with AAV8-hAAT-moFGF21 vectors as young adults (top panel) or as adults (bottom panel).

FIG. 18B Circulating levels of FGF21 at different time-points after vector administration.

FGF21 labels in the figure refer to moFGF21 in accordance with this Figure legend. Data information: All values are expressed as mean±SEM. In (FIGS. 18A-18B) n=7-10 animals/group. * P<0.05, ** P<0.01 and *** P<0.001 versus the chow-fed Null-injected group. #P<0.05, ##P<0.01 and ###P<0.001 versus the HFD-fed Null-injected group. HFD, High-fat diet.

FIGS. 19A-19E. FGF21 gene transfer to the liver counteracts HFD-induced obesity.

FIGS. 19A, 19B Representative images of animals belonging to all experimental groups of the studies performed in young adults (FIG. 19A) or in adults (FIG. 19B).

FIG. 19C Representative images of the epididymal white adipose (eWAT) pad obtained at sacrifice from animals treated with several doses of AAV8-hAAT-moFGF21 as young adults (left) or adults (right).

Representative images of the liver obtained from animals treated as FIG. 19D young adults (left) or adults (right).

FIG. 19E AAV-derived FGF21 expression in the liver of animals treated as young adults or adults. The qPCR was performed with primers that specifically detected the codon-optimized murine FGF21 (coFGF21) coding sequence.

FGF21 labels in the figure refer to moFGF21 in accordance with this Figure legend. Data information: All values are expressed as mean±SEM. In (FIG. 19E) n=7-10 animals/group. HFD, High-fat diet. ND, non-detected.

FIGS. 20A-20E. AAV8-hAAT-moFGF21-mediated increased energy expenditure and decreased fat accumulation in iBAT and iWAT.

FIG. 20A Assessment of the locomotor activity through the Open field test in animals that had been subjected to HFD-feeding since ˜2 months of age and were treated with either null or FGF21-encoding vectors 2 months later (young adults).

FIG. 20B Hematoxylin-eosin staining of iBAT tissue sections obtained from animals treated as young adults (left) or adults (right).

FIG. 20C Western-blot analysis of UCP1 content in iBAT from the same cohort of animals as in (FIG. 20A). A representative immunoblot is shown (left). The histogram depicts the densitometric analysis of two different immunoblots (right).

FIG. 20D Hematoxylin-eosin staining of iWAT tissue sections obtained from animals treated as young adults (left) or adults (right).

FIG. 20E Quantification by qRT-PCR of the expression of Phosphol in iWAT in the groups of animals that initiated the HFD feeding and received FGF21 vectors as young adults or adults.

FGF21 labels in the figure refer to moFGF21 in accordance with this Figure legend. Data information: All values are expressed as mean±SEM. In (FIGS. 20A-20C) n=7-10 animals/group. In (FIG. 20E) n=4 animals/group. In (G) n=7-10 animals/group. * P<0.05, ** P<0.01 and *** P<0.001 versus the chow-fed Null-injected group. #P<0.05 and ###P<0.001 versus the HFD-fed Null-injected group. HFD, High-fat diet.

FIGS. 21A-21C. Energy expenditure 10 months after gene transfer to the liver.

FIG. 21A Energy expenditure was measured 10 months after AAV8-hAAT-null or AAV8-hAAT-moFGF21 vector delivery in the cohort of animals that initiated HFD-feeding at 2 months of age. Data were taken during the light and dark cycles.

FIG. 21B Western-blot analysis of UCP1 content in iWAT from the same cohort of animals. A representative immunoblot is shown (left). The graph shows the densitometric analysis of two different immunoblots (right).

FIG. 21C Relative expression of Serca2b and RyR2 in the iWAT of the groups of animals that initiated the HFD feeding and received FGF21 vectors as young adults or adults

FGF21 labels in the figure refer to moFGF21 in accordance with this Figure legend. Data information: All values are expressed as mean±SEM. In (FIG. 21A) n=7-10 animals/group. In (FIG. 21B) n=4 animals/group. In (FIG. 21C) n=7-10 animals/group. *P<0.05, ** P<0.01 and *** P<0.001 versus the chow-fed Null-injected group. ###P<0.001 versus the HFD-fed Null-injected group. HFD, High-fat diet.

FIGS. 22A-22D. AAV8-hAAT-moFGF21-mediated reversal of islet hyperplasia.

FIG. 22A Fasted glucagon levels in the group of animals that initiated the HFD feeding and received FGF21 vectors as young adults.

FIG. 22B β-cell mass in the group of animals that initiated the HFD feeding and received FGF21 vectors as adults.

FIG. 22C Representative images of the immunostaining against insulin in pancreas sections from animals that received 5×10¹⁰ vg/mouse of AAV8-hAAT-moFGF21 as adults. Scale bars: 400 μm. Inset scale bars: 100 μm.

FIG. 22D Representative images of the double immunostaining against insulin (dark grey) and glucagon (light grey) in pancreas sections from animals that received 5×10¹⁰ vg/mouse of AAV8-hAAT-moFGF21 as young adults (upper panel) or adults (lower panel). Scale bars: 100 μm.

FGF21 labels in the figure refer to moFGF21 in accordance with this Figure legend. Data information: All values are expressed as mean±SEM. In (FIGS. 22A-22C) n=7-10 animals/group. In (FIG. 22D) n=4-5 animals/group. * P<0.05, ** P<0.01 and *** P<0.001 versus the chow-fed Null-injected group. #P<0.05, ##P<0.01 and ###P<0.001 versus the HFD-fed Null-injected group. HFD, High-fat diet.

FIGS. 23A-23B. Treatment with AAV8-hAAT-moFGF21 improves glucose tolerance.

FIG. 23A Glucose tolerance was studied in the group of mice that initiated the HFD feeding and received FGF21 vectors as young adults after an intraperitoneal injection of glucose (2 g/kg body weight).

FIG. 23B Serum insulin levels during the glucose tolerance test shown in (FIG. 23A).

Data information: All data represent the mean mean±SEM. In (FIGS. 23A-23B) n=7-10 animals/group. * P<0.05, ** P<0.01 and *** P<0.001 versus the chow-fed Null-injected group. #P<0.05, ##P<0.01 and ###P<0.001 versus the HFD-fed Null-injected group. HFD, High-fat diet.

FIGS. 24A-24H. Reversal of WAT hypertrophy and inflammation by AAV8-hAAT-moFGF21 treatment.

FIG. 24A Representative images of the hematoxylin-eosin staining of the eWAT from animals fed a chow or a HFD and administered with either AAV8-hAAT-null or 5×10¹⁰ vg/mouse AAV8-hAAT-moFGF21 vectors as young adults (left panels) or adults (right panels). While HFD-fed, null-injected mice had larger adipocytes, HFD-fed, FGF21-treated animals had adipocytes of reduced size. Scale bars: 100 μm.

FIG. 24B Morphometric analysis of the area of WAT adipocytes in animals treated as young adults or as adults.

FIGS. 24C, 24D Circulating levels of adiponectin (FIG. 24C) and leptin (FIG. 24D).

FIG. 24E Immunohistochemistry for the macrophage-specific marker Mac2 in eWAT sections from animals that received 5×10¹⁰ vg/mouse AAV8-hAAT-moFGF21 as adults. The micrographs illustrate the presence of crown-like structures (arrows and inset) in the eWAT of HFD-fed, null-injected animals but no in the eWAT of HFD-fed, FGF21-treated mice. Scale bars: 200 μm and 50 μm (inset).

FIGS. 24F-24H Quantification by qRT-PCR of the expression of the markers of inflammation F4/80 (FIG. 24F), IL1-β (FIG. 24G) and TNF-α (FIG. 24H) in the group of animals that initiated the HFD feeding and received FGF21 vectors as adults.

FGF21 labels in the figure refer to moFGF21 in accordance with this Figure legend. Data information: All values are expressed as mean±SEM. In (FIG. 24B) n=4 animals/group. In (FIGS. 24F-24H) n=7-10 animals/group. * P<0.05, ** P<0.01 and *** P<0.001 versus the chow-fed Null-injected group. #P<0.05, ##P<0.01 and ###P<0.001 versus the HFD-fed Null-injected group. HFD, High-fat diet.

FIGS. 25A-25E. Adipocyte size and inflammation in AAV8-hAAT-moFGF21-treated animals.

FIG. 25A Frequency distribution of adipocyte area in the groups of animals that initiated the chow or HFD feeding and received either AAV8-hAAT-null or 5×10¹⁰ vg/mouse AAV8-hAAT-moFGF21 vectors as young adults (top graph) or adults (bottom graph).

FIG. 25B Mac2 immunohistochemistry in eWAT of animals in which the study was initiated as young adults. The crown-like structures formed by infiltrating macrophages in the eWAT of HFD-fed, null-injected mice are indicated by arrows. Scale bars: 200 μm and 50 μm (inset).

FIGS. 25C-25E Relative expression by qRT-PCR of the markers of inflammation F4/80, CD68 and TNF-α in the same cohort of animals as in (FIG. 25B).

FGF21 labels in the figure refer to moFGF21 in accordance with this Figure legend. Data information: All values are expressed as mean±SEM. In (FIG. 25A) n=4 animals/group. In (FIGS. 25C-25E) n=7-10 animals/group. *** P<0.001 versus the chow-fed Null-injected group. ###P<0.001 versus the HFD-fed Null-injected group. HFD, High-fat diet.

FIGS. 26A-26D. Treatment with FGF21-encoding vectors reverses hepatic steatosis and inflammation.

FIG. 26A Representative images of the hematoxylin-eosin staining of liver sections obtained from animals fed a chow or a HFD and administered with either AAV8-hAAT-null or 5×10¹⁰ vg/mouse AAV8-hAAT-moFGF21 vectors. HFD clearly induced the deposition of lipid droplets in the liver, and this was reverted by AAV8-hAAT-moFGF21 treatment both in young adults and in adults. Scale bars: 100 μm.

FIGS. 26B, 26C Fed hepatic triglyceride and cholesterol content in the same cohorts of animals.

FIG. 26D Immunostaining for the macrophage-specific marker Mac-2 of liver sections from animals fed a HFD that received either AAV8-hAAT-null or 5×10¹⁰ vg/mouse AAV8-hAAT-moFGF21 vectors. Arrows indicate the presence of crown-like structures. Scale bars: 200 μm and 50 μm (inset).

FGF21 labels in the figure refer to moFGF21 in accordance with this Figure legend.

Data information: All values are expressed as mean±SEM. In (FIGS. 26B-26C) n=7-10 animals/group. ** P<0.01 and *** P<0.001 versus the chow-fed Null-injected group. ##P<0.01 versus the HFD-fed Null-injected group. HFD, High-fat diet.

FIG. 27. AAV8-hAAT-moFGF21-mediated amelioration of liver fibrosis.

Analysis of hepatic fibrosis through Masson's trichrome staining in animals fed a HFD that received 5×10¹⁰ vg/mouse of either AAV8-hAAT-null or AAV8-hAAT-moFGF21 vectors. AAV8-hAAT-moFGF21 treatment (right panels) markedly decreased the detection of collagen fibers that were readily detectable (in blue) in animals treated with the null vector (left panels). Scale bars: 50 μm. FGF21 labels in the figure refer to moFGF21 in accordance with this Figure legend.

FIGS. 28A-28C. AAV8-hAAT-moFGF21 treatment improves liver fibrosis.

FIG. 28A Analysis of hepatic fibrosis through PicroSirius staining in animals fed a HFD that received 5×10¹⁰ vg/mouse of either AAV8-hAAT-null or AAV8-hAAT-moFGF21 vectors. AAV8-hAAT-moFGF21 treatment (right panels) markedly decreased the detection of collagen fibers that were readily detectable (in black) in animals treated with the null vector (left panels). Scale bars: 50 μm.

FIGS. 28B, 28C Quantification by qRT-PCR of the expression of collagen 1 in the liver in the group of animals that initiated the HFD feeding and received FGF21 vectors as young adults (FIG. 28B) or adults (FIG. 28C).

FGF21 labels in the figure refer to moFGF21 in accordance with this Figure legend. Data information: All values are expressed as mean±SEM. In (FIGS. 28B-28C) n=7-10 animals/group. * P<0.05, ** P<0.01 and *** P<0.001 versus the chow-fed Null-injected group. “P<0.05 and ###P<0.001 versus the HFD-fed Null-injected group. HFD, High-fat diet.

FIGS. 29A-29Q. No bone abnormalities were observed in AAV8-hAAT-moFGF21-treated animals. The long-term effects of FGF21 gene transfer on bones were studied by comparison of HFD-fed mice treated with the highest dose (5×10¹⁰ vg/mouse) of AAV8-hAAT-moFGF21 vectors as young adults or adults with null-injected, chow or HFD-fed animals.

FIG. 29A Total naso-anal length.

FIG. 29B Tibial length.

FIGS. 29C-29O Micro-computed tomography (μCT) analysis of the epiphysis (FIGS. 29C-29J) and the diaphysis (FIGS. 29K-290) of tibiae obtained at the time of sacrifice, i.e. when animals were 18 months of age, from HFD-fed mice administered with either null or FGF21-encoding AAV vectors.

FIGS. 29P, 29Q Circulating IGFBP1 (FIG. 29P) and IGF1 (FIG. 29Q) levels measured by ELISA.

FGF21 labels in the figure refer to moFGF21 in accordance with this Figure legend. Data information: All data represent the mean±SEM. In (FIGS. 29A, 29P-29Q) n=7-10 animals/group. In (FIGS. 29B-290) n=4 animals/group. ** P<0.01 and *** P<0.001 versus the chow-fed Null-injected group. HFD, High-fat diet; BMD, bone mineral density; BMC, bone mineral content; BV, bone volume; BV/TV, bone volume/tissue volume ratio; BS/BV, bone surface/bone volume ratio; Tb.N, trabecular number; Tb.Th, trabecular thickness; Tb.Sp, trabecular separation.

FIG. 30. Analysis of glycaemic profiles in C57B16 mice treated with AAV8-hAAT-moFGF21 vectors. Blood glucose levels were evaluated under fed conditions. AAV, IV administration of 5×10¹⁰ vg or 2×10¹¹ vg of AAV8-hAAT-moFGF21 (n=13 and 15, respectively) or 2×10¹¹ vg of AAV8-null vectors (n=15). STZ, treatment with streptozotocin (5×50 mg/kg). Results shown are means+SEM. * p<0.05; *** p<0.001 vs AAV8-hAAT-Null. FGF21 labels in the figure refer to moFGF21 in accordance with this Figure legend.

FIGS. 31A-31H. Gene transfer of FGF21 to the skeletal muscle of healthy animals.

FIG. 31A Circulating levels of FGF21 measured 40 weeks after injection of 3×10¹¹ vg/mouse of either AAV1-CMV-Null or AAV1-CMV-moFGF21 vectors to the skeletal muscle of healthy animals fed a chow diet.

FIG. 31B AAV-derived FGF21 expression in the muscles and liver of healthy animals injected intramuscularly with AAV1-CMV-Null or AAV1-CMV-moFGF21 vectors.

FIG. 31C Evolution of the body weight in the 40-week follow-up period.

FIG. 31D Wet tissue weight of different muscles, adipose pads and liver.

FIGS. 31E, 31F Hepatic triglyceride and cholesterol content in the fed state.

FIG. 31G Fed serum insulin levels.

FIG. 31H Insulin sensitivity assessed through intraperitoneal injection of insulin (0.75 units/kg body weight) and represented as percentage of initial blood glucose.

FGF21 labels in the figure refer to moFGF21 in accordance with this Figure legend. Data information: All values are expressed as mean±SEM. In (FIGS. 31A-31H) n=5-7 animals/group. * P<0.05, ** P<0.01 and *** P<0.001 versus the Null-injected group.

FIGS. 32A-32F. AAV1-mediated skeletal muscle gene transfer of FGF21 counteracts HFD-induced obesity and insulin resistance.

FIGS. 32A, 32B Evolution of body weight (FIG. 32A) and body weight gain (FIG. 32B) in animals treated with AAV1-CMV-moFGF21. C57B16 mice were fed a HFD for ˜12 weeks and then administered with 3×10¹¹ vg/mouse of AAV1-CMV-moFGF21 vectors. Control obese mice and control chow-fed mice received 3×10¹¹ vg of AAV1-CMV-null.

FIG. 32C Circulating levels of FGF21 at different time-points after vector administration.

FIGS. 32D, 32E Fasted blood glucose (FIG. 32D) and fed serum insulin (FIG. 32E) levels in the same groups of animals as in (FIGS. 32A, 32B).

FIG. 32F Insulin sensitivity was determined in all experimental groups after an intraperitoneal injection of insulin (0.75 units/kg body weight). Results were calculated as the percentage of initial blood glucose levels.

FGF21 labels in the figure refer to moFGF21 in accordance with this Figure legend. Data information: All values are expressed as mean±SEM. In (FIGS. 32A-32F) HFD-fed mice n=10 animals/group; chow-fed mice n=5 animals/group. *** P<0.001 versus the HFD-fed null-injected group.

FIG. 33. In vivo increased FGF21 circulating levels by codon-optimization of nucleotide sequences encoding human FGF21. Circulating levels of hFGF21 in C57B16 mice administered hydrodynamically with plasmids encoding wild-type hFGF21 or three different variants of codon-optimized human FGF21 sequences. Results are expressed as the mean±SEM. n=9-10 mice/group. ND, non detected. Negative control, untreated mice. * p<0.05 vs Non-treated mice.

FIGS. 34A-34E. In vitro increased FGF21 expression levels by hAAT-moFGF21, CAG-moFGF21-doublemiRT and CMV-moFGF21 expression cassettes. (FIG. 34A) Expression levels of FGF21 in HEK293 cells transfected with plasmids encoding the WT murine FGF21 coding sequence under the control of the EF1a promoter (EF1a-mFGF21) or a codon-optimized murine FGF21 coding sequence under the control of the CMV promoter (CMV-moFGF21) or of the CAG promoter in conjunction with four tandem repeats of the miRT122a sequence and four tandems repeats of the miRT1 sequence (CAG-moFGF21-doublemiRT). (FIGS. 34B and 34C) Intracellular FGF21 protein content (FIG. 34B) and FGF21 protein levels in the culture medium (FIG. 34C) in the same cells as in (FIG. 34A). (FIG. 34D) Expression levels of FGF21 in C2C12 cells transfected with plasmids encoding the WT murine FGF21 coding sequence under the control of the EF1a promoter (EF1a-mFGF21) or a codon-optimized murine FGF21 coding sequence under the control of the CMV promoter (CMV-moFGF21). (FIG. 34E) Expression levels of FGF21 in HepG2 cells transfected with plasmids encoding the WT murine FGF21 coding sequence under the control of the EF1a promoter (EF1a-mFGF21) or a codon-optimized murine FGF21 coding sequence under the control of the hAAT promoter (hAAT-moFGF21). The qPCR was performed with primers that detected both the wt and the codon-optimized FGF21 coding sequences. Results are expressed as the mean±SEM. n=3 wells/group. ND, non detected. * p<0.05 vs control. ###p<0.001 vs EF1a-mFGF21.

FIGS. 35A-35B. In vivo increased hepatic FGF21 expression and FGF21 circulating levels by hAAT-moFGF21 and CMV-moFGF21 expression cassettes. (FIG. 35A) Expression levels of FGF21 in the liver of C57B16 mice hydrodynamically administered with plasmids encoding the WT murine FGF21 coding sequence under the control of the elongation factor 1a (EF1a) promoter (EF1a-mFGF21) or a codon-optimized murine FGF21 coding sequence under the control of the CMV promoter (CMV-moFGF21) or the hAAT promoter (hAAT-moFGF21). The qPCR was performed with primers that detected both the wt and the codon-optimized FGF21 coding sequences. (FIG. 35B) FGF21 circulating levels in the same cohorts as in (FIG. 35A). Results are expressed as the mean±SEM. n=5 mice/group. ** p<0.01 vs. EF1a-mFGF21

FIGS. 36A-36B. In vivo increased hepatic FGF21 expression and FGF21 circulating levels by AAV8-hAAT-moFGF21. (FIG. 36A) Expression levels of FGF21 in the liver of C57B16 mice intravenously administered with 1×10¹⁰ vg, 2×10¹⁰ vg or 5×10¹⁰ vg of AAV8 vectors encoding the WT murine FGF21 coding sequence under the control of the elongation factor 1a (EF1a) promoter (AAV8-EF1a-mFGF21) or a codon-optimized murine FGF21 coding sequence under the control of the hAAT promoter (AAV8-hAAT-moFGF21). The qPCR was performed with primers that detected both the wt and the codon-optimized FGF21 coding sequences. (FIG. 36B) FGF21 circulating levels in the same cohorts as in (FIG. 36A). Analyses were performed two weeks post-AAV. Results are expressed as the mean±SEM. n=4-5 mice/group. Control, untreated mice. ** p<0.01 and *** p<0.001 vs. control. ##p<0.01 and ###p<0.001 vs. AAV8-EF1a-mFGF21

FIGS. 37A-37B. In vivo increased adipose FGF21 expression by AAV8-CAG-moFGF21-dmiRT. (FIGS. 37A-37B) Expression levels of FGF21 in the eWAT (FIG. 37A) or the liver (FIG. 37B) of C57B16 mice administered intra-eWAT with 2×10¹⁰ vg, 5×10¹⁰ vg or 1×10¹¹ vg of either AAV8 vectors encoding the WT murine FGF21 coding sequence under the control of the elongation factor 1a (EF1a) promoter (AAV8-EF1a-mFGF21) or AAV8 vectors encoding a codon-optimized murine FGF21 coding sequence under the control of the CAG promoter in conjunction with four tandem repeats of the miRT122a sequence and four tandems repeats of the miRT1 sequence (AAV8-CAG-moFGF21-doublemiRT). The qPCR was performed with primers that detected both the wt and the codon-optimized FGF21 coding sequences. Analyses were performed two weeks post-AAV. Results are expressed as the mean±SEM. n=4-5 mice/group. Control, untreated mice. eWAT, epidydimal white adipose tissue. * p<0.05, ** p<0.01 and

FIGS. 38A-38B. In vivo increased FGF21 expression in the skeletal muscle by AAV1-CMV-moFGF21. (FIGS. 38A-38B) Expression levels of FGF21 in the quadriceps (FIG. 38A) or the liver (FIG. 38B) of C57B16 mice administered intramuscularly with 5×10¹⁰ vg, 1×10¹¹ vg or 3×10¹¹ vg of either AAV8 vectors encoding the WT murine FGF21 coding sequence under the control of the elongation factor 1a (EF1a) promoter (AAV8-EF1a-mFGF21) or AAV1 vectors encoding a codon-optimized murine FGF21 coding sequence under the control of the CMV promoter (AAV1-CMV-FGF21). The qPCR was performed with primers that detected both the wt and the codon-optimized FGF21 coding sequences. Analyses were performed two weeks post-AAV. Results are expressed as the mean±SEM. n=4-5 mice/group. Control, untreated mice. * p<0.05, ** p<0.01 and *** p<0.001 vs. control. #p<0.05, ##p<0.01 and ###p<0.001 vs. AAV8-EF1a-mFGF21.

### EXAMPLES

### General Procedures to the Examples

### Subject Characteristics

Male C57Bl/6J mice and B6.V-Lep^{ob}/OlaHsd (ob/ob) mice were used. Mice were fed ad libitum with a standard diet (2018S Teklad Global Diets®, Harlan Labs., Inc., Madison, WI, US) or a high fat diet (TD.88137 Harlan Teklad Madison, WI, US) and kept under a light-dark cycle of 12 h (lights on at 8:00 a.m.) and stable temperature (22° C.±2). For tissue sampling, mice were anesthetized by means of inhalational anesthetic isoflurane (IsoFlo®, Abbott Laboratories, Abbott Park, IL, US) and decapitated. Tissues of interest were excised and kept at −80° C. or with formalin until analysis. All experimental procedures were approved by the Ethics Committee for Animal and Human Experimentation of the Universitat Autónoma de Barcelona.

### Recombinant AAV Vectors

Single-stranded AAV vectors of serotype 1, 8 or 9 were produced by triple transfection of HEK293 cells according to standard methods (Ayuso, E. et al, 2010. Curr Gene Ther. 10 (6): 423-36). Cells were cultured in 10 roller bottles (850 cm², flat; Corning™, Sigma-Aldrich Co., Saint Louis, MO, US) in DMEM 10% FBS to 80% confluence and co-transfected by calcium phosphate method with a plasmid carrying the expression cassette flanked by the AAV2 ITRs, a helper plasmid carrying the AAV2 rep gene and the AAV of serotypes 1, 8 or 9 cap gene, and a plasmid carrying the adenovirus helper functions. Transgenes used were: murine, canine or human codon-optimized or wt FGF21 coding-sequence driven by 1) the cytomegalovirus (CMV) early enhancer/chicken beta actin (CAG) promoter with the addition of four tandem repeats of the miRT122a sequence (5′CAAACACCATTGTCACACTCCA3′) (SEQ ID NO:12) and four tandems repeats of the miRT1 sequence (5′TTACATACTTCTTTACATTCCA3′) (SEQ ID NO:13) cloned in the 3′ untranslated region of the expression cassette; 2) the CMV promoter; or 3) the human al-antitrypsin promoter (hAAT). Noncoding plasmids carrying the CAG, hAAT or CMV promoters were used to produce null vectors. AAV were purified with an optimized method based on a polyethylene glycol precipitation step and two consecutive cesium chloride (CsCl) gradients. This second-generation CsCl-based protocol reduced empty AAV capsids and DNA and protein impurities dramatically (Ayuso, E. et al., 2010. Curr Gene Ther. 10(6):423-36). Purified AAV vectors were dialyzed against PBS, filtered and stored at −80° C. Titers of viral genomes were determined by quantitative PCR following the protocol described for the AAV2 reference standard material using linearized plasmid DNA as standard curve (Lock M, et al., Hum. Gene Ther. 2010; 21:1273-1285). The vectors were constructed according to molecular biology techniques well known in the art.

### In Vivo Intra-eWAT Administration of AAV Vectors

Mice were anesthetized with an intraperitoneal injection of ketamine (100 mg/kg) and xylazine (10 mg/kg). A laparotomy was performed in order to expose the epididymal white adipose tissue. AAV vectors were resuspended in PBS with 0.001% Pluronic® F68 (Gibco) and injected directly into the epididymal fat pad. Each epididymal fat pad was injected twice with 50 μL of the AAV solution (one injection close to the testicle and the other one in the middle of the fat pad). The abdomen was rinsed with sterile saline solution and closed with a two-layer approach.

Systemic administration of AAV vectors

The appropriate amount of the AAV solution was diluted in 200 μL of PBS with 0.001% Pluronic® and was manually injected into the lateral tail vein without exerting pressure at the moment of delivery. Before the injection, the animals were put under a 250 W infrared heat lamp (Philips NV, Amsterdam, NL) for a few minutes to dilate the blood vessels and facilitate viewing and easier access to the tail vein. A plastic restrainer (Harvard Apparatus, Holliston, MA, US) was used to secure the animal for injection. No anesthesia was used since an appropriate restraining device was employed. A 30-gauge needle was utilized to inject the animals.

### Intramuscular Administration of AAV Vectors

Mice were anesthetized with an intraperitoneal injection of ketamine (100 mg/kg) and xylazine (10 mg/kg). Hind limbs were shaved and vectors were administered by intramuscular injection in a total volume of 180 μl divided into six injection sites distributed in the quadriceps, gastrocnemius, and tibialis cranealis of each hind limb.

### Immunohistochemical and Morphometric Analysis

Tissues were fixed for 24 h in formalin (Panreac Química), embedded in paraffin, and sectioned. Tissue samples were stained with hematoxylin-eosin. Adipocyte area was determined in 12 hematoxylin/eosin WAT images per animal taken at 10× with the Nikon Eclipse E800 microscope (Nikon, Tokyo, Japan) connected to a videocamera with a monitor with an image analysis software (analySIS 3.0; Soft Imaging System, Center Valley, PA, EEUU) and each adipocyte area was quantified in μm². Mean adipocyte area was calculated for each experimental group and distribution of adipocytes according to size categories was represented in a histogram. Four animals per group were used and at least 250 adipocytes per animal were analyzed.

### Immunohistochemistry

Tissues were fixed for 12-24 h in 10% formalin, embedded in paraffin and sectioned. Sections were incubated overnight at 4° C. with rat anti-Mac2 (1:50; CL8942AP; Cedarlane), guinea pig anti-insulin (1:100; I-8510; Sigma-Aldrich) or rabbit anti-glucagon (1:100; 219-01; Signet Labs). Biotinylated rabbit anti-rat (1:300; E0467; Dako), goat anti-rabbit IgG (Alexa Fluor 568-conjugated) (1:200; A11011; ThermoFisher), goat anti-guinea pig IgG (Alexa Fluor 488-conjugated) (1:300; A11073; ThermoFisher) or rabbit anti-guinea pig coupled to peroxidase (1:300; P0141; Dako) were used as secondary antibodies. The ABC peroxidase kit (Pierce) was used for immunodetection, and sections were counterstained in Mayer's hematoxylin. Hoechst (B2261; Sigma-Aldrich) was used for nuclear counterstaining of fluorescent specimens. PicroSirius Red staining and Masson's trichrome staining were used to evaluate fibrosis. The percentage of β-cell area in the pancreas was analyzed in two insulin-stained sections 200 μm apart, by dividing the area of all insulin+ cells in one section by the total pancreas area of that section. β-cell mass was calculated by multiplying pancreas weight by percentage of β-cell area, as previously described (Jimenez et al, 2011).

### RNA Analysis

Total RNA was obtained from adipose depots or liver by using QIAzol Lysis Reagent (Qiagen NV, Venlo, NL) or Tripure isolation reagent (Roche Diagnostics Corp., Indianapolis, IN, US), respectively, and RNeasy Lipid Tissue Minikit (Qiagen NV, Venlo, NL). In order to eliminate the residual viral genomes, total RNA was treated with DNAsel (Qiagen NV, Venlo, NL). For RT-PCR, 1 μg of RNA samples was reverse-transcribed using Transcriptor First Strand cDNA Synthesis Kit (04379012001, Roche, California, USA). Real-time quantitative PCR was performed in a SmartCyclerII® (Cepheid, Sunnyvale, USA) using EXPRESS SYBRGreen qPCR supermix (Invitrogen™, Life Technologies Corp., Carslbad, CA, US). Data was normalized with Rplp0 values and analyzed as previously described (Pfaffl, M., Nucleic Acids Res. 2001; 29 (9): e45).

### Hormone and Metabolite Assays

Blood glucose levels were measured with a Glucometer Elite™ analyzer (Bayer, Leverkusen, Germany). Circulating levels of FGF21 were determined by quantitative sandwich enzyme immunoassay Mouse/Rat FGF-21 ELISA kit (MF2100, R&Dsystems, Abingdon, UK). Serum insulin concentrations were determined by Rat Insulin ELISA sandwich assay (90010, Crystal Chem INC. Downers Grove, IL 60515, USA). To extract lipids from tissue, frozen samples of approximately 100 mg were weighted and homogenized in 15 ml chloroform:methanol (2:1). Lipid and aqeuous phases were then separated by adding 3 ml of H₂SO₄ 0.05% and keeping them overnight at 4° C. Once the phases were separated, the aqueous superior phase was eliminated using a Pasteur pipet and 1 ml of the inferior lipid phase was recuperated in a glass tube. 1 ml of a chloroform and Triton X-100 at 1% solution was added to the glass tube and it was incubated at 90° C. in a bath, to evaporate the chloroform. By the use of the chloroform and Triton X-100 mix, any remaining aqeuous particle was eliminated from the lipid phase. After the evaporation, chloroform was rinsed to the walls of the tube to concentrate the sample and, it was warmed again at 90° C. to evaporate the chloroform. Once the sediment was completely dry and concentrated, it was resuspended by the addition of 500 μl of H20 miliQ at 37° C. The amount of triglycerides was finally determined using the commercial product GPO-PAP (Roche Diagnostics, Basel, Switzerland). Serum triglycerides and cholesterol were quantified spectrophotometrically using an enzymatic assay kit (Horiba-ABX, Montpellier, France). All biochemical parameters were determined using Pentra 400 Analyzer (Horiba-ABX).

Glycemia was determined using a Glucometer Elite™ (Bayer). Glucagon levels were measured using a glucagon Radioimmunoassay (#GL-32K, EMD Millipore). Adiponectin, leptin, IGFBP1 and IGF1 were determined using the Mouse Adiponectin ELISA kit (80569, Crystal Chem), the Mouse Leptin ELISA kit (90030, Crystal Chem), the IGFBP1 (Mouse) ELISA kit (KA3054, Abnova) and the m/r IGF-I-ELISA kit (E25, Mediagnost), respectively.

### Insulin Tolerance Tests

For insulin tolerance tests, insulin (0.75 IU/kg body wt; Humulin Regular; Eli Lilly, Indianapolis, IN) was injected intraperitoneally into awake fed mice. Glucose concentration was determined in blood samples obtained from the tail vein at the indicated time points after the insulin injection.

### Glucose Tolerance Test

Awake mice were fasted overnight (16 h) and administered with an intraperitoneal injection of glucose (2 g/kg body weight). Glycemia was measured in tail vein blood samples at the indicated time points. Venous blood was collected from tail vein in tubes (Microvette®) CB 300, SARSTEDT) at the same time points and immediately centrifuged to separate serum, which was used to measure insulin levels.

### Oximetry

An indirect open circuit calorimeter (Oxylet, Panlab, Cornellà, Spain) was used to monitor oxygen consumption, carbon dioxide production in eight metabolic chambers simultaneously. Mice were individualized and acclimated to the metabolic chambers for 24 h, and data were collected every 15 min for 3 min in each cage for other 24 h. Data were taken from the light and dark cycle and adjusted for body weight. To calculate energy expenditure the Metabolism software provided by the manufacturer was used.

### Transfection of HEK293, C2C12 and HepG2 Cells

Cells were cultured in a 24-well plate and transfected with 0.8 ug of DNA per well using Lipofectamine 2000 following the manufacturer's instructions (Thermo Fisher Scientific).

### Bone Analysis

Bone volume and architecture were evaluated by μCT. Mouse tibiae were fixed in neutral buffered formalin (10%) and scanned using the explore Locus CT scanner (General Electric) at 27-micron resolution. Trabeculae were analyzed in 1 mm3 of proximal tibial epiphysis and 1.8 mm3 of cortical tibial diaphysis in 4 mice/group. Bone parameters were calculated with the Micro View 3D Image Viewer & Analysis Tool. The length of the tibia was measured from the intercondilar eminence to the medial malleolus.

### Western Blot Analysis

iWAT and iBAT were homogenized in QIAzol Lysis Reagent (Qiagen) and the protein fraction was isolated from the organic phase following the manufacturer's instructions. Proteins were separated by 12% SDS-PAGE, and analyzed by immunoblotting with rabbit polyclonal anti-UCP1 (ab10983; Abcam) and rabbit polyclonal anti-a-tubulin (ab4074; Abcam) antibodies. Detection was performed using ECL Plus detection reagent (Amersham Biosciences).

### Open Field Test

The open field test was performed between 9:00 am and 1:00 pm as previously reported (Haurigot et al, 2013). Briefly, animals were placed in the center of a brightly lit chamber (41×41×30 cm) crossed by 2 bundles of photobeams (LE 8811; Panlab) that detect horizontal and vertical movements. Motor and exploratory activities were evaluated during the first 6 minutes. The total distance covered was evaluated using a video tracking system (SMART Junior; Panlab).

### Statistical Analysis

All values are expressed as mean±SEM. Differences between groups were compared by Student's t-test. Differences were considered significant at p<0.05.

### Examples

### Example 1. Prevention of Obesity and Diabetes by Intra-eWAT Administration of AAV-CAG-moFGF21-dmiRT Vectors in C57B16 Mice

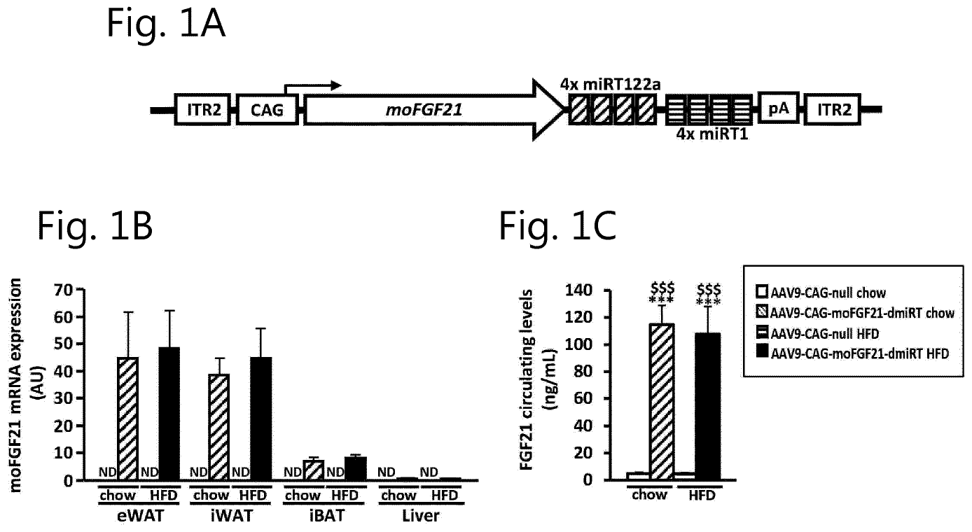

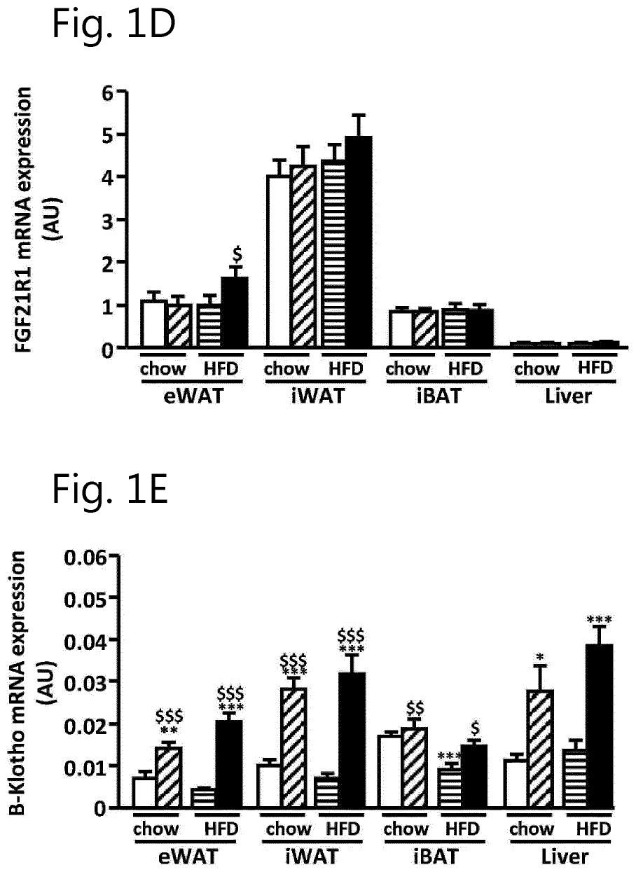

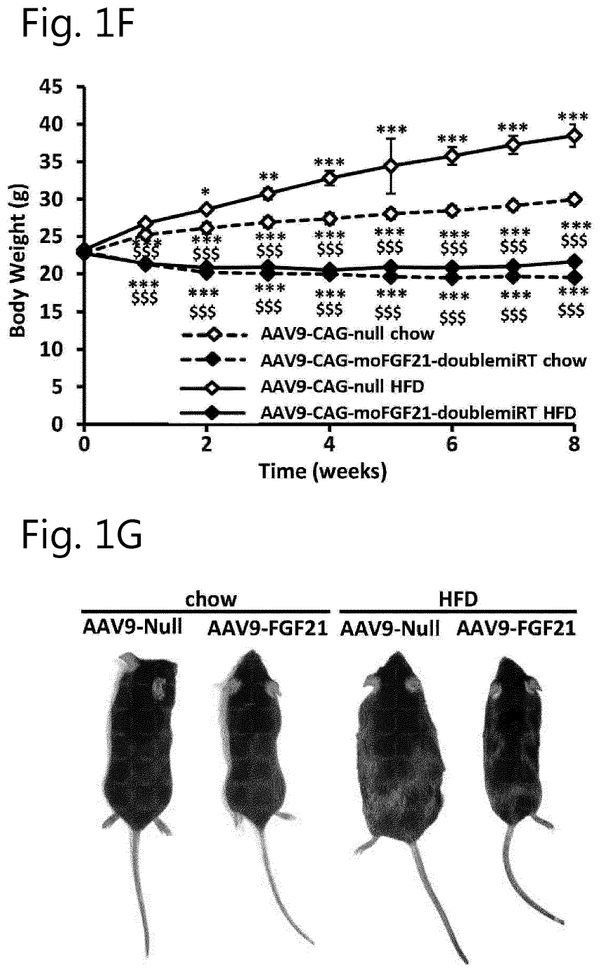

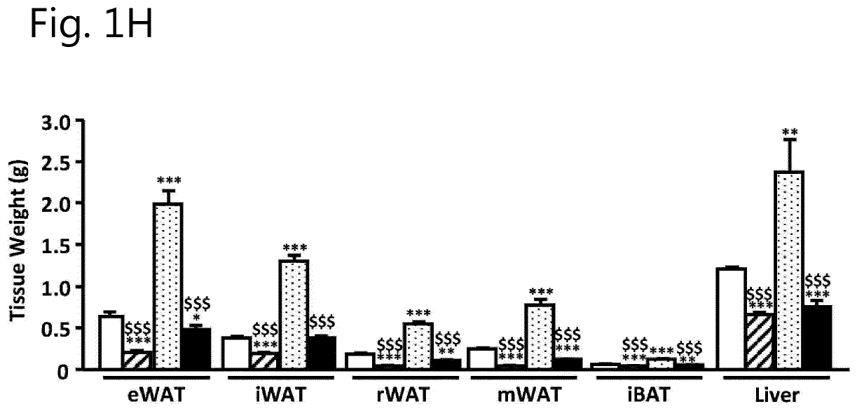

We evaluated the therapeutic potential of the AAV-mediated genetic engineering of adipose tissue with FGF21 to prevent obesity and diabetes in 8-week-old male C57B16. Intra-eWAT (eWAT: epididymal white adipose tissue) administration of 10¹² viral genomes (vg) of AAV9 vectors encoding a murine codon-optimized FGF21 coding sequence under the control of the CAG ubiquitous promoter which included target sites of miR122 and miR1 (AAV9-CAG-moFGF21-doublemiRT) (FIG. 1A) mediated adipose-specific overexpression of FGF21 (FIG. 1B) as well as high secretion of the protein into the bloodstream (FIG. 1C). AAV9-CAG-moFGF21-doublemiRT-treated mice also showed overexpression of the FGF21 receptor1 (FGF21R1) in eWAT (FIG. 1D) and β-Klotho (a FGF21 co-receptor) in adipose tissue and liver (FIG. 1E) in comparison with AAV9-CAG-null vectors (vectors that retain equal infectivity but do not encode any transgene). The CAG-moFGF21-doublemiRT construct is comprised in SEQ ID NO: 32 and the CAG-null construct is comprised in SEQ ID NO:31.

Following AAV-mediated gene transfer of FGF21 to eWAT, mice fed a chow diet showed loss of body weight (FIGS. 1F and 1G). When challenged with a high fat diet (HFD), animals overexpressing FGF21 in adipose tissue remained lean for the duration of the experiment whereas AAV9-CAG-null treated mice became progressively obese (FIGS. 1F and 1G). According to their lower body weight, both chow- and HFD-fed AAV9-CAG-moFGF21-doublemiRT-treated mice showed decreased weight of adipose depots and liver (FIG. 1H).

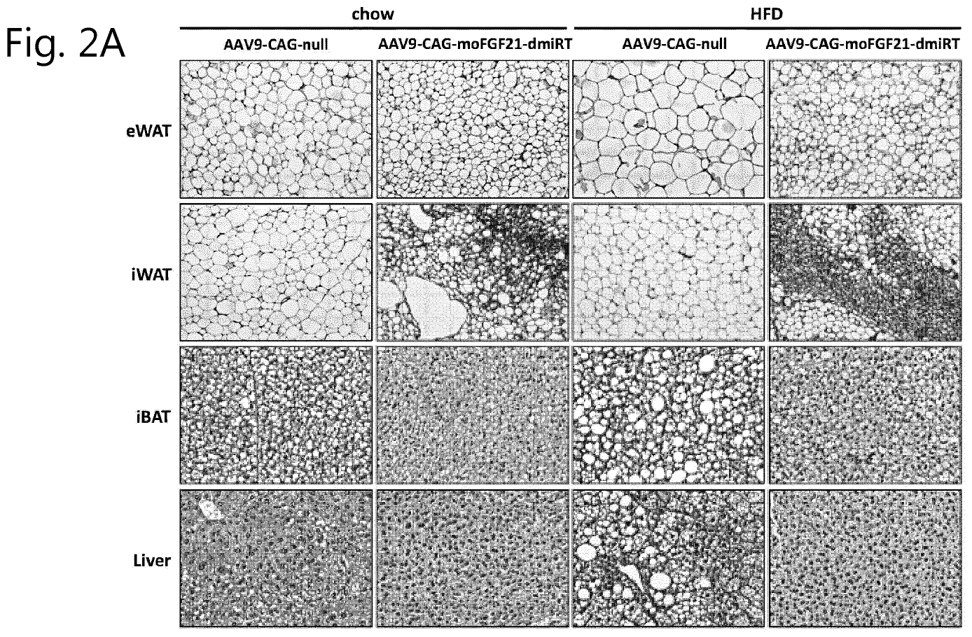

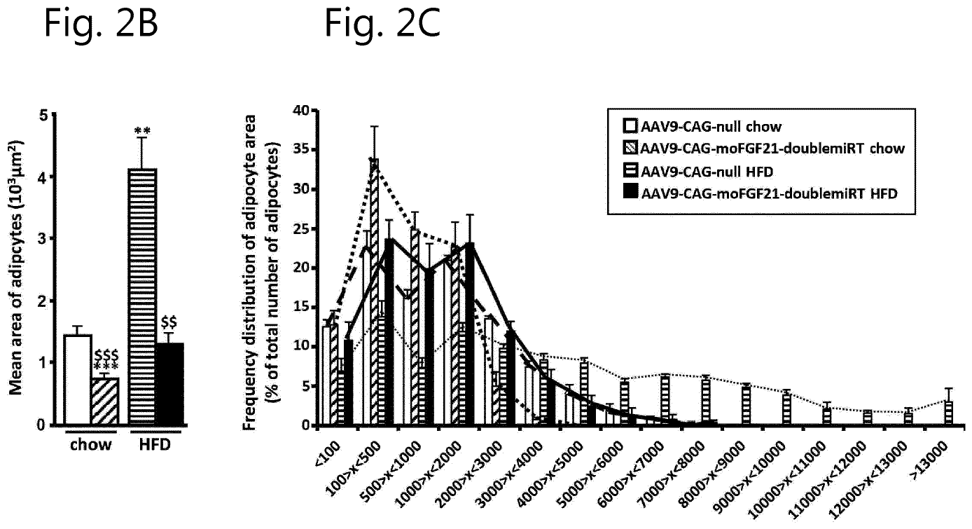

Histological analysis of white adipose tissue by hematoxylin-eosin staining revealed decreased white adipocyte size in eWAT and iWAT (iWAT: inguinal white adipose tissue) and multiple multilocular adipocytes in iWAT, suggesting that browning of this depot had occurred (FIG. 2A). Morphometric analysis further confirmed decreased mean area of white adipocytes in AAV9-CAG-moFGF21-doublemiRT-treated mice (FIG. 2B).

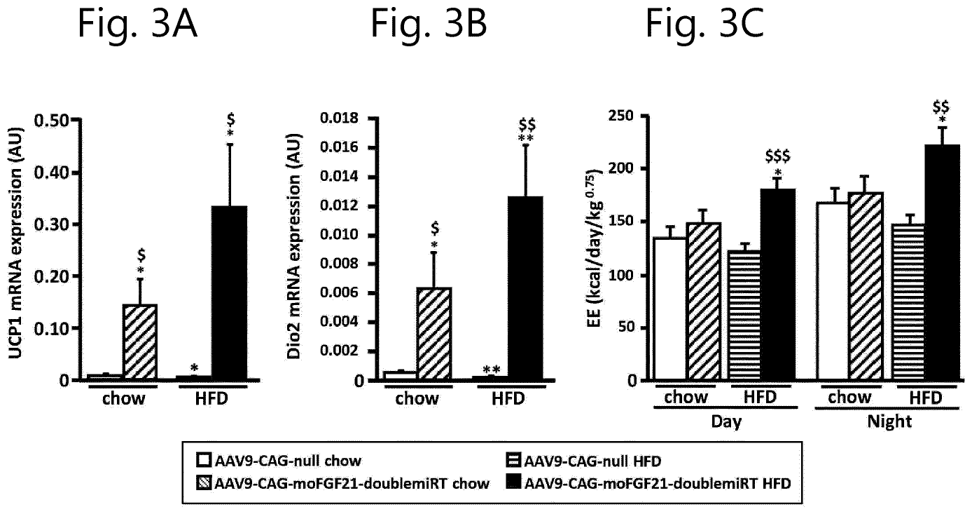

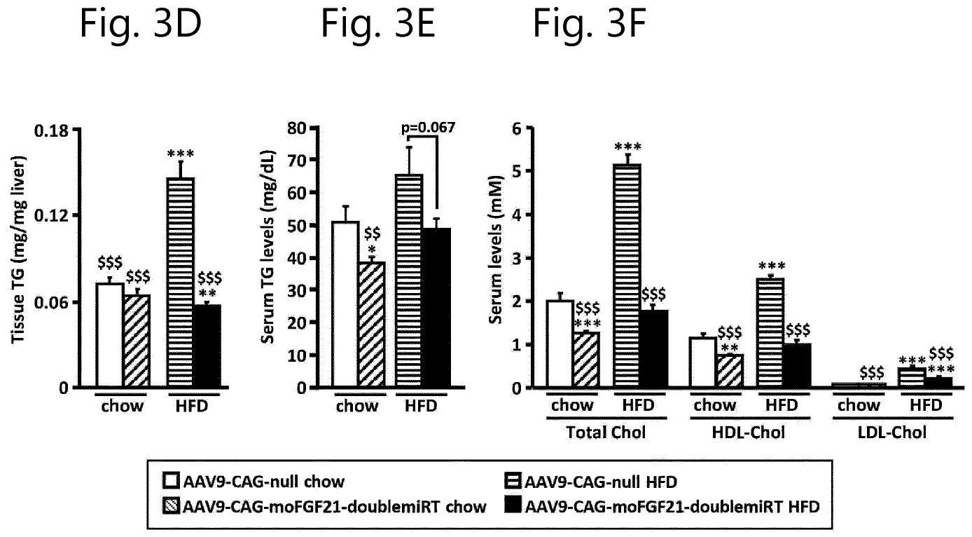

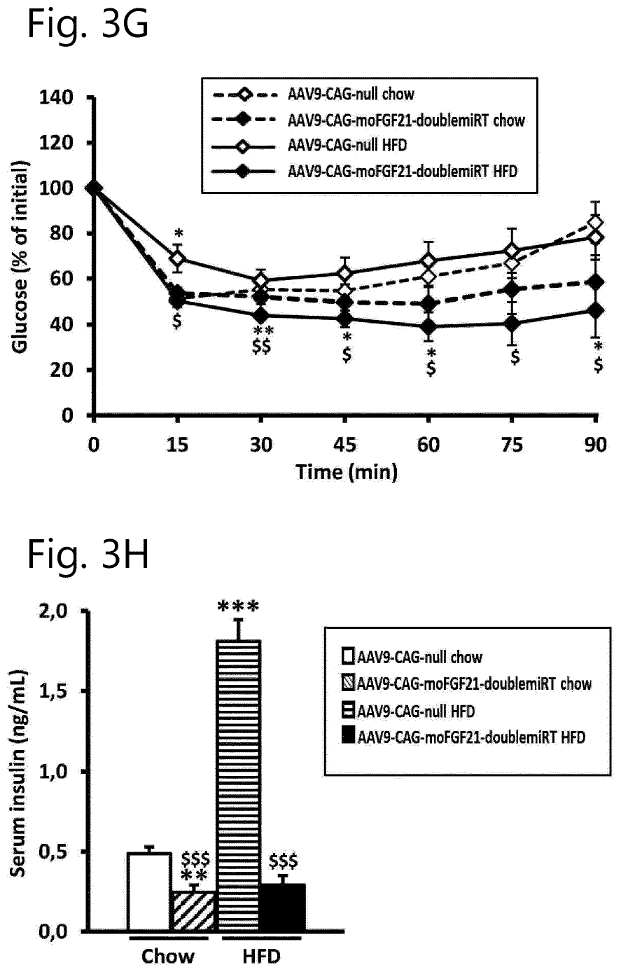

The frequency distribution of the area of white adipocytes was also different between groups. Both chow- and HFD-fed AAV9-CAG-moFGF21-doublemiRT-treated mice presented increased number of small adipocytes and fewer big adipocytes (FIG. 2C). Noticeably, the frequency distribution of the area of white adipocytes in HFD-fed AAV9-CAG-moFGF21-doublemiRT-treated mice was almost identical to that of chow-fed AAV9-CAG-null-treated animals (FIG. 2C). Therefore, the HFD-induced hypertrophy of adipocytes observed in AAV9-CAG-null-treated mice was blocked in mice overexpressing FGF21. Overexpression of UCP1 and Dio2 in iWAT (FIGS. 3A and 3B) further confirmed browning of iWAT in chow- and HFD-fed AAV9-CAG-moFGF21-doublemiRT-treated mice.

Histologic analysis of iBAT (iBAT: interscapular brown adipose tissue) showed lower lipid accumulation in this depot in chow- and HFD-fed AAV9-CAG-moFGF21-doublemiRT-treated mice in comparison with AAV9-CAG-null mice (FIG. 2A). According to this result and to browning of iWAT, energy expenditure (FIG. 3C) of HFD-fed AAV9-CAG-moFGF21-doublemiRT-treated mice during the light and dark cycles was higher than that of HFD-fed AAV9-CAG-null mice. Altogether, these data suggest that AAV9-CAG-moFGF21-doublemiRT-treated mice have increased thermogenic activity.

Liver histologic sections showed decreased lipid accumulation in hepatocytes of mice overexpressing FGF21 compared with AAV9-CAG-null-treated mice both under chow or HFD (FIG. 2A). Accordingly, HFD-fed AAV9-CAG-moFGF21-doublemiRT-treated mice normalized their hepatic content of triglycerides (TG) (FIG. 3D). In parallel, circulating levels of TG, total cholesterol, HDL-cholesterol and LDL-cholesterol were normalized in HFD-fed mice overexpressing FGF21 (FIGS. 3E and 3F).

HFD-fed mice overexpressing FGF21 were more insulin sensitive than HFD-fed AAV9-null treated mice (FIG. 3G) and both chow- and HFD-fed AAV9-CAG-moFGF21-doublemiRT-treated mice showed decreased insulin circulating levels in comparison with their AAV9-CAG-null treated counterparts (FIG. 3H).

### Example 2. Reversion of Obesity and Improvement of Glucose Metabolism by Intra-eWAT Administration of AAV-CAG-moFGF21-dmiRT Vectors in Ob/Ob Mice

We evaluated the anti-diabetic and anti-obesogenic therapeutic potential of the AAV-mediated genetic engineering of adipose tissue with FGF21 in 11-week-old male ob/ob mice, which have defective leptin signalling and are a widely used genetic model of obesity and diabetes. To this end, a dose-response study was performed. Ob/ob mice were administered locally into the eWAT with four different doses (10¹⁰ vg, 5×10¹⁰ vg, 2×10¹¹ vg or 10¹² vg) of AAV8-CAG-moFGF21-doublemiRT vectors (FIG. 1A). As control, ob/ob animals were administered intra-eWAT with 10¹² vg of AAV8-CAG-null vectors.

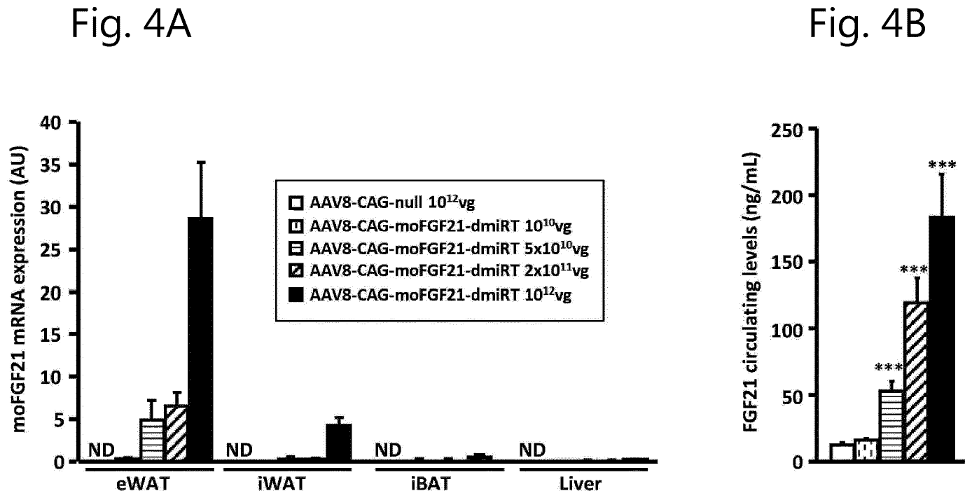

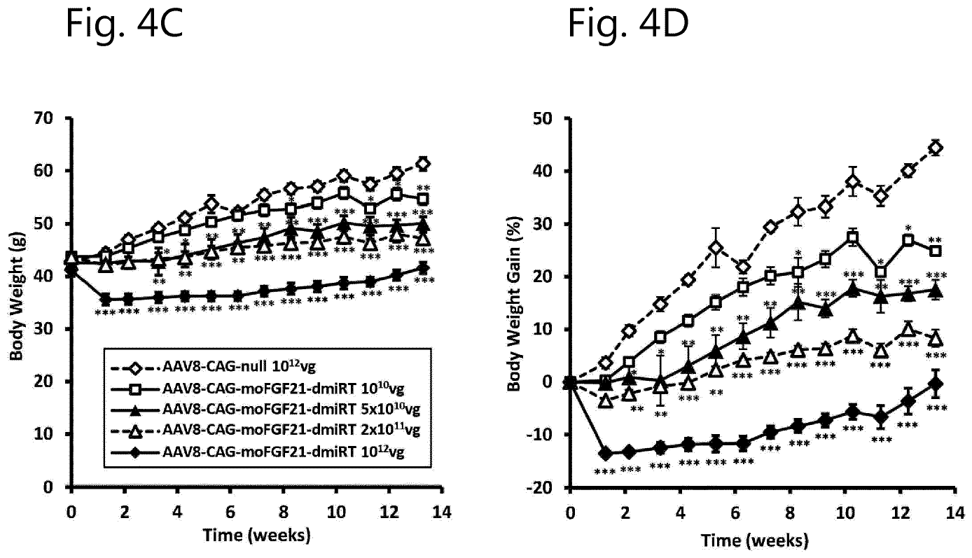

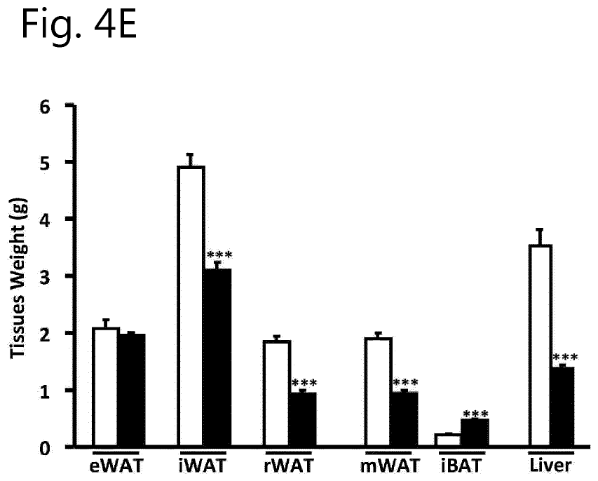

Intra-eWAT administration of AAV8-CAG-moFGF21-doublemiRT vectors mediated specific overexpression of FGF21 in white adipose tissue as well as high secretion of the protein into the bloodstream in a dose-dependent manner (FIGS. 4A and 4B). Specifically, the dose of 10¹² vg of AAV8-CAG-moFGF21-doublemiRT vectors mediated a very robust overexpression of FGF21 in eWAT and iWAT (FIG. 4A) and achieved the highest circulating FGF21 levels (FIG. 4B). In contrast, the lowest dose administered, 10¹⁰ vg of AAV8-CAG-moFGF21-doublemiRT vectors, only produced a very modest overexpression of FGF21 in eWAT (FIG. 4A) and animals treated with this dose showed no differences in the serum FGF21 levels in comparison with AAV8-CAG-null treated animals (FIG. 4B), probably because FGF21 acted in a paracrine-autocrine manner. Accordingly, whereas AAV8-CAG-null-treated animals progressively increased their body weight, animals treated with AAV8-CAG-moFGF21-doublemiRT vectors showed decreased body weight gain proportional to the dose of vectors administered (FIGS. 4C and 4D). Noticeably, animals treated with 10¹² vg of AAV8-CAG-moFGF21-doublemiRT vectors lost approximately 15% of weight during the first two weeks after AAV administration and afterwards increased their body weight until they reached the initial body weight (FIGS. 4C and 4D). Thus, animals administered intra-eWAT with 10¹² vg of AAV8-CAG-moFGF21-doublemiRT vectors showed a 40% difference in total body weight in comparison with AAV8-CAG-null-treated animals at the end of the experiment (FIG. 4D). In agreement, animals treated with 10¹² vg of AAV8-CAG-moFGF21-doublemiRT vectors showed marked decreased adiposity and a 60% reduction of the weight of the liver (FIG. 4E). iBAT weight was increased in this cohort of mice (FIG. 4E), probably due to increased thermogenic activity.

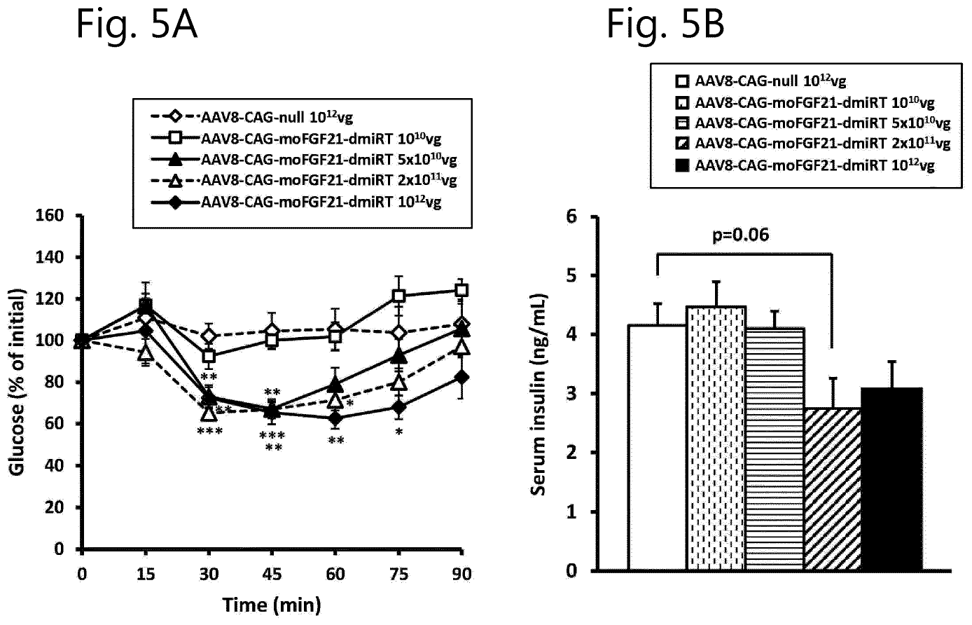

Animals treated with 5×10¹⁰ vg, 2×10¹¹ vg or 10¹² vg of AAV8-CAG-moFGF21-doublemiRT vectors presented improved insulin sensitivity in comparison with AAV8-CAG-null-treated mice (FIG. 5A). Animals treated with 2×10¹¹ vg or 10¹² vg of AAV8-CAG-moFGF21-doublemiRT vectors also showed lower insulin circulating levels than ob/ob mice treated with AAV8-CAG-null vectors (FIG. 5B).

### Example 3. Reversion of Obesity and Improvement of Glucose Metabolism by Intravenous Administration of AAV-hAAT-moFGF21 Vectors in Ob/Ob Mice

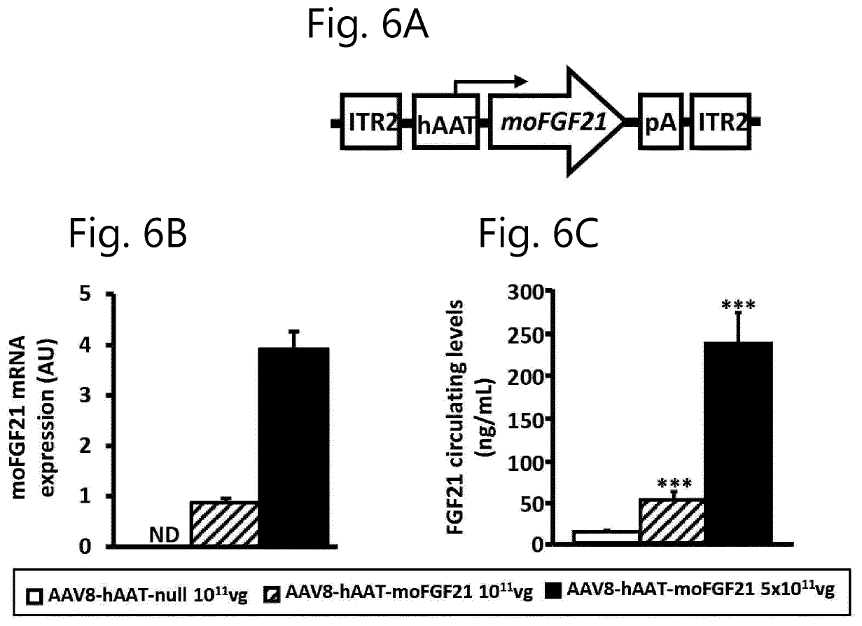

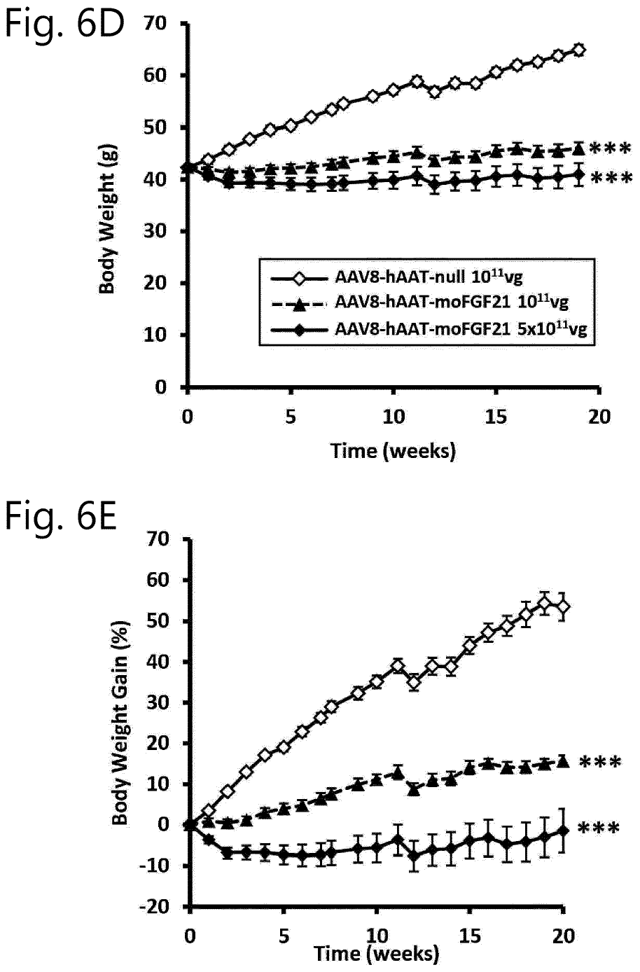

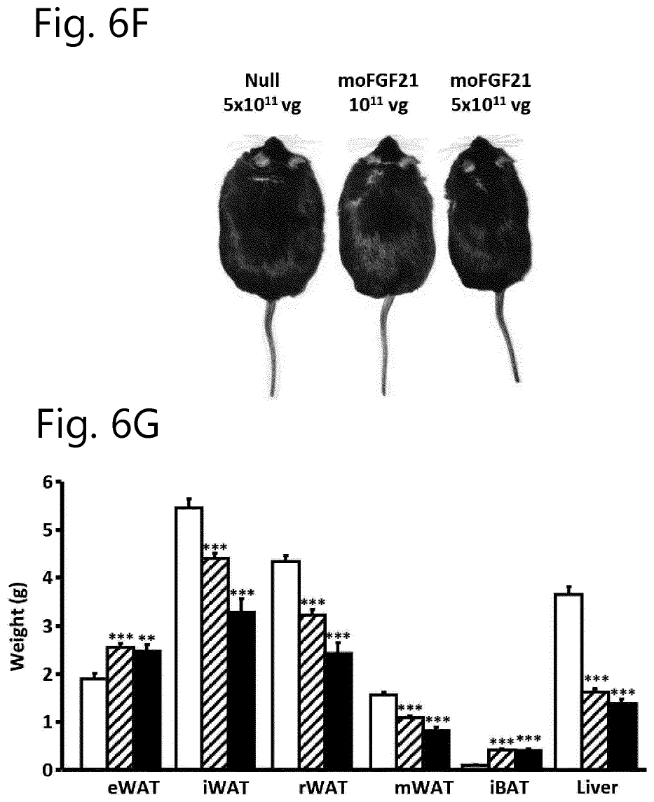

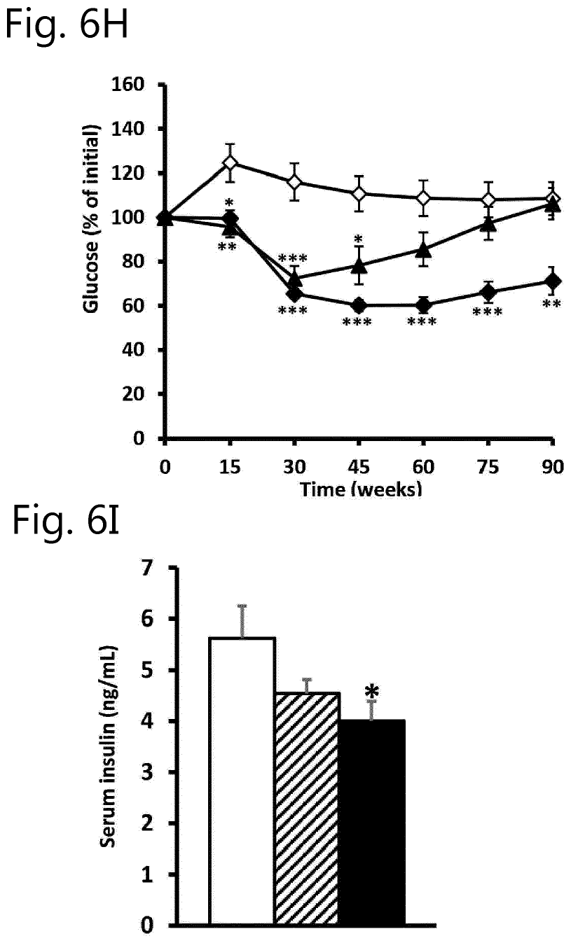

We also evaluated the anti-diabetic and anti-obesogenic effects mediated by the increased circulating levels of FGF21 by means of AAV-mediated genetic engineering of the liver in 8-week-old male ob/ob mice. Ob/ob mice were administered intravenously (IV) with 10¹¹ vg or 5×10¹¹ vg of AAV8 vectors encoding a murine codon-optimized FGF21 coding sequence under the control of the liver-specific human al-antitrypsin (hAAT) promoter (AAV8-hAAT-moFGF21) (FIG. 6A). As control, ob/ob animals were administered IV with 5×10¹¹ vg of AAV8-hAAT-null vectors. The hAAT-moFGF21 construct is comprised in SEQ ID NO: 34 and the hAAT-null construct is comprised in SEQ ID NO:33.

Intravenous administration of AAV8-hAAT-moFGF21 vectors mediated specific overexpression of FGF21 in the liver as well as high secretion of the protein into the bloodstream in a dose-dependent manner (FIGS. 6B and 6C). Specifically, the dose of 5x 10¹¹ vg of AAV8-hAAT-moFGF21 vectors mediated a very robust overexpression of FGF21 in the liver (FIG. 6B) and achieved the highest circulating FGF21 levels (FIG. 6C). The body weight of animals treated with this dose of AAV8-hAAT-moFGF21 vectors decreased approximately 7% during the two first weeks after AAV administration and afterwards slightly increased whereas AAV8-hAAT-null-treated mice progressively put on weight (FIGS. 6D, 6E and 6F). Mice administered with 10¹¹ vg of AAV8-hAAT-moFGF21 vectors gained markedly much less weight than AAV8-hAAT-null-treated animals (FIGS. 6D, 6E and 6F). Specifically, AAV8-hAAT-null animals showed a 50% increase in their body weight at the end of the experiment in comparison with the 10% weight gain of animals treated with 10¹¹ vg of AAV8-hAAT-moFGF21 vectors (FIG. 6E). According to their lower body weight, animals overexpressing FGF21 in the liver showed significant decreased adiposity, particularly in those animals treated with the highest dose of vectors, and approximately a 60% reduction of the liver weight (FIG. 6G). iBAT weight was similarly increased in both groups of AAV8-hAAT-moFGF21-treated mice (FIG. 6G), probably due to higher thermogenic activity in these animals in comparison with mice administered with AAV8-hAAT-null vectors.

Animals treated with AAV8-hAAT-moFGF21 vectors showed improved insulin sensitivity and decreased insulin circulating levels in comparison with AAV8-hAAT-null-treated mice (FIGS. 6H and 61)

### Example 4. Long-Term Reversion of Obesity and Diabetes by Intravenous Administration of AAV-hAAT-moFGF21 Vectors in HFD-Fed Mice

We also evaluated the anti-diabetic and anti-obesogenic effects mediated by the increased circulating levels of FGF21 by means of AAV-mediated genetic engineering of the liver in obese C57B16 mice. Nine-week-old male C57B16 mice (young adults) were fed a HFD for 9 weeks and then administered IV with 10¹⁰ vg or 5×10¹⁰ vg of AAV8-hAAT-moFGF21 vectors (FIG. 6A). After AAV administration, AAV8-hAAT-moFGF21-treated mice were maintained on HFD for 52 weeks. As controls, 5×10¹⁰ vg of AAV8-hAAT-null were administered IV to chow- and HFD-fed C57B16 mice. These two latter cohorts of mice were maintained either on chow diet or HFD thereafter.

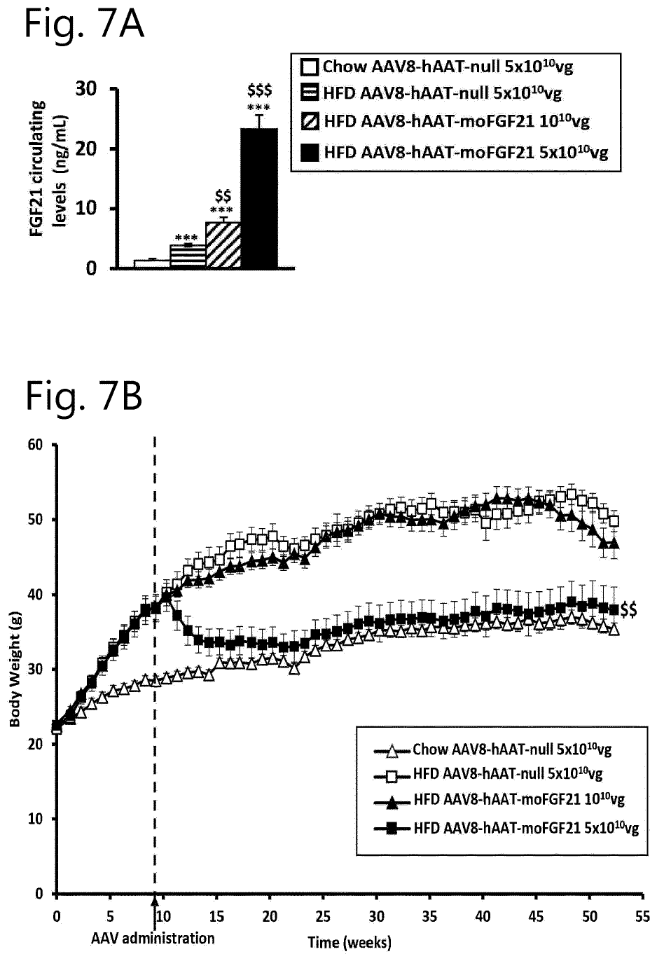

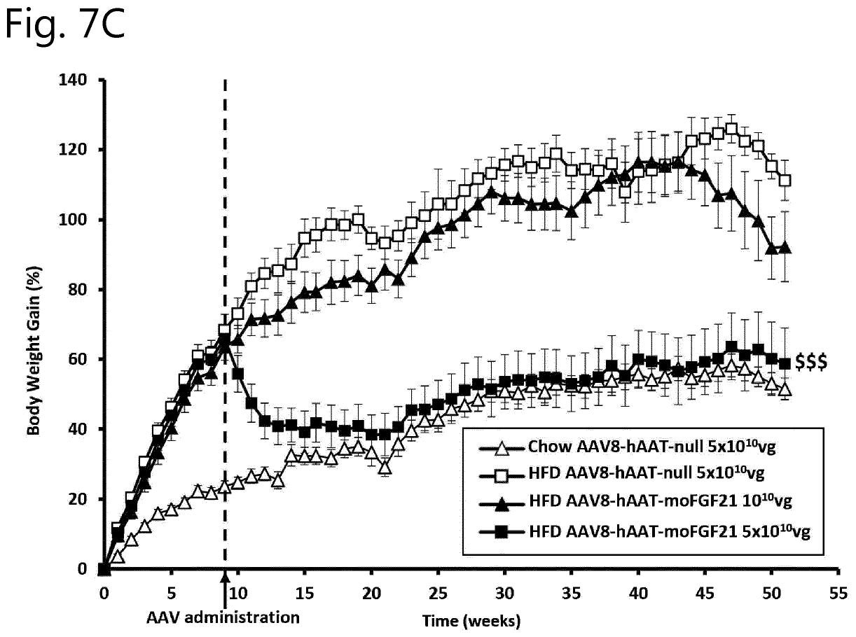

Intravenous administration of AAV8-hAAT-moFGF21 vectors in HFD-fed mice mediated high secretion of FGF21 into the bloodstream in a dose-dependent manner (FIG. 7A).

No differences in body weight were observed between HFD-fed AAV8-null-treated mice and HFD-fed animals administered with 10¹⁰ vg of AAV8-hAAT-moFGF21 vectors (FIGS. 7B and 7C). However, HFD-fed animals treated with 5×10¹⁰ vg of AAV8-hAAT-moFGF21 vectors initially lost 20% of body weight after AAV administration and then progressively gained weight similarly to chow-fed AAV8-hAAT-null-treated mice (FIGS. 7B and 7C). Noticeably, from week 9 after AAV administration onwards no statistical significant differences were observed in the total body weight and body weight gain between HFD-fed animals administered with 5×10¹⁰ vg of AAV8-hAAT-moFGF21 vectors and chow-fed AAV8-hAAT-null-treated mice (FIGS. 7B and 7C).

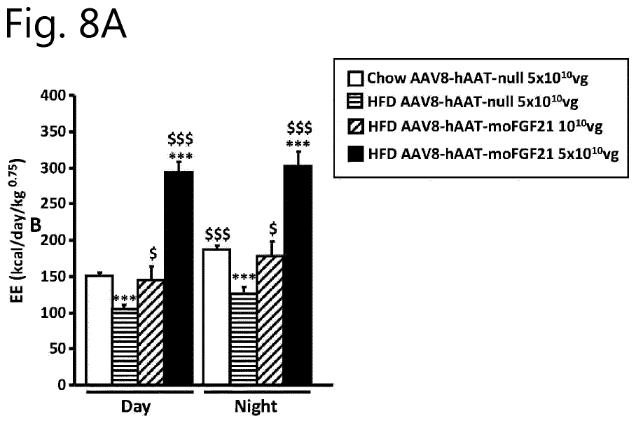

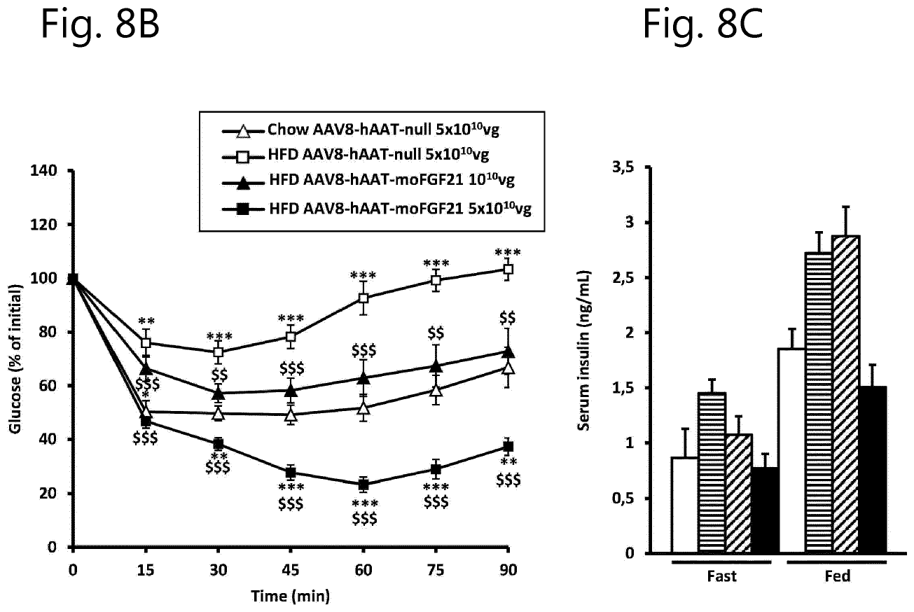

The energy expenditure of HFD-fed mice treated with 5×10¹⁰ vg of AAV8-hAAT-moFGF21 vectors during the light and dark cycles was higher than that of chow- and HFD-fed AAV8-hAAT-null mice (FIG. 8A). No differences in energy expenditure were observed among chow- and HFD-fed AAV8-hAAT-null-treated animals and mice administered with 10¹⁰ vg of AAV8-hAAT-moFGF21 vectors (FIG. 8A). Altogether, these data suggest that mice treated with 5×10¹⁰ vg of AAV8-hAAT-moFGF21 have increased thermogenic activity.

Animals treated with 10¹⁰ vg of AAV8-hAAT-moFGF21 vectors presented improved insulin sensitivity in comparison with HFD-fed mice administered with AAV8-hAAT-null vectors and their insulin sensitivity was similar to that of chow-fed mice treated with AAV8-hAAT-null vectors (FIG. 8B). Noticeably, animals administered with 5×10¹⁰ vg of AAV8-hAAT-moFGF21 vectors presented improved insulin sensitivity in comparison with chow-fed mice administered with AAV8-hAAT-null vectors and showed normalized insulin circulating levels (FIGS. 8B and 8C).

### Example 5. Reversion of Obesity and Diabetes by Intravenous Administration of AAV-hAAT-moFGF21 Vectors in Old HFD-Fed Mice

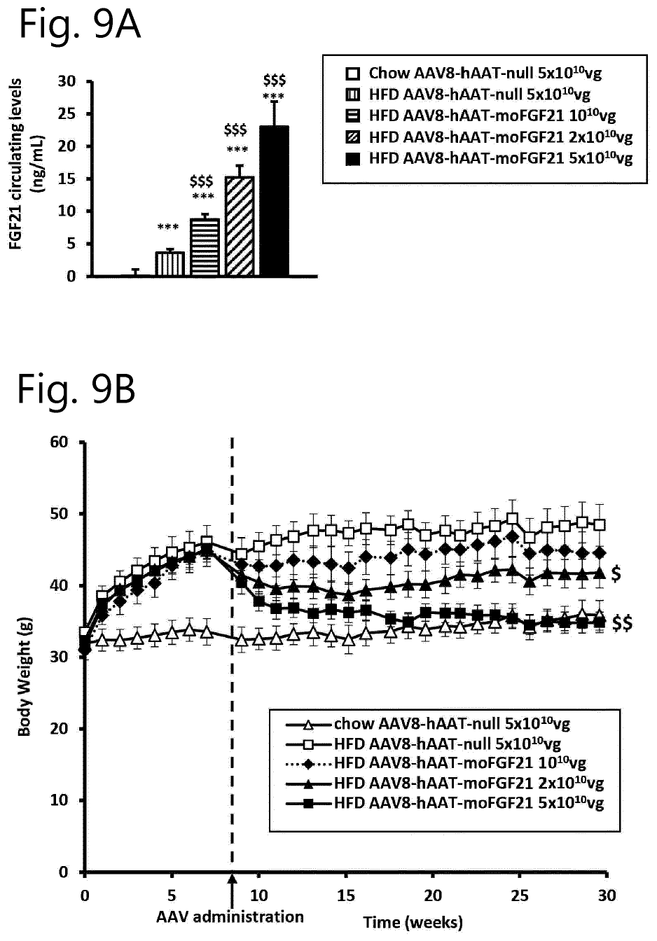

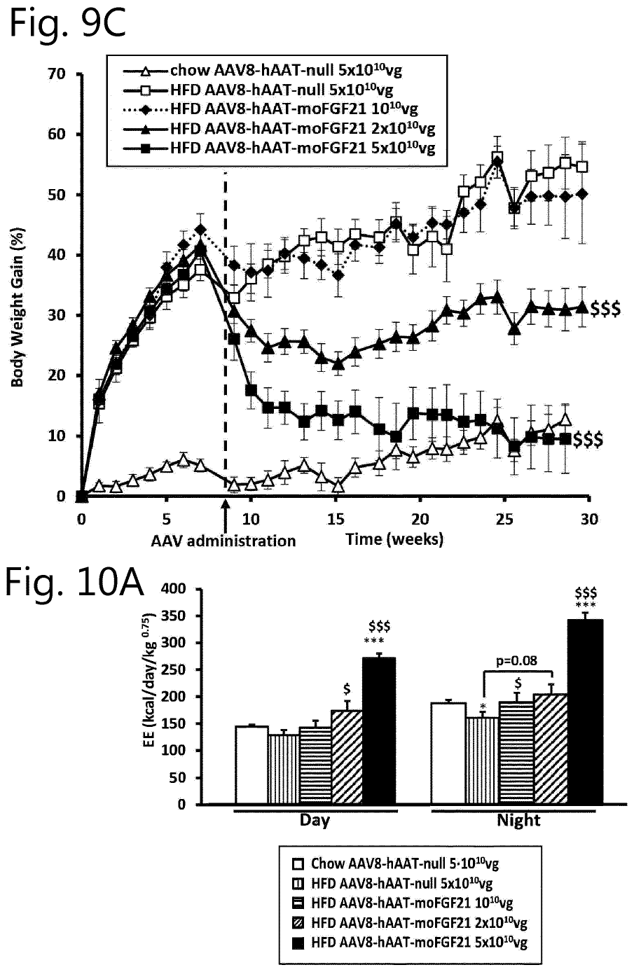

We also evaluated the anti-diabetic and anti-obesogenic effects of FGF21 in obese old (adults) C57B16 mice. Seven and a half-month-old male C57B16 mice were fed a HFD for 8 weeks and then administered IV with 10¹⁰ vg, 2×10¹⁰ vg or 5×10¹⁰ vg of AAV8-hAAT-moFGF21 vectors (FIG. 6A). After AAV administration, AAV8-hAAT-moFGF21-treated mice were maintained on HFD for 22 weeks. As controls, 5×10¹⁰ vg of AAV8-hAAT-null were administered IV to chow- and HFD-fed old C57B16 mice. These two latter cohorts of mice were maintained either on chow diet or HFD thereafter. Intravenous administration of AAV8-hAAT-moFGF21 vectors in old HFD-fed mice mediated high secretion of FGF21 into the bloodstream in a dose-dependent manner (FIG. 9A).

No differences in body weight were observed between HFD-fed AAV8-null-treated mice and HFD-fed animals administered with 10¹⁰ vg of AAV8-hAAT-moFGF21 vectors (FIGS. 9B and 9C). However, HFD-fed animals treated with either 2×10¹⁰ vg or 5×10¹⁰ vg of AAV8-hAAT-moFGF21 vectors initially lost 15 and 20%, respectively, of body weight after AAV administration (FIGS. 9B and 9C). Thereafter, animals treated with 2×10¹⁰ vg of AAV8-hAAT-moFGF21 vectors progressively gained weight similarly to chow-fed AAV8-hAAT-null-treated mice whereas no significant changes in body weight of animals treated with 5×10¹⁰ vg of AAV8-hAAT-moFGF21 vectors were observed (FIGS. 9B and 9C). Noticeably from week 3 after AAV administration onwards no statistical significant differences were observed in the total body weight and body weight gain between HFD-fed animals administered with 5×10¹⁰ vg of AAV8-hAAT-moFGF21 vectors and chow-fed AAV8-hAAT-null-treated mice (FIGS. 9B and 9C).

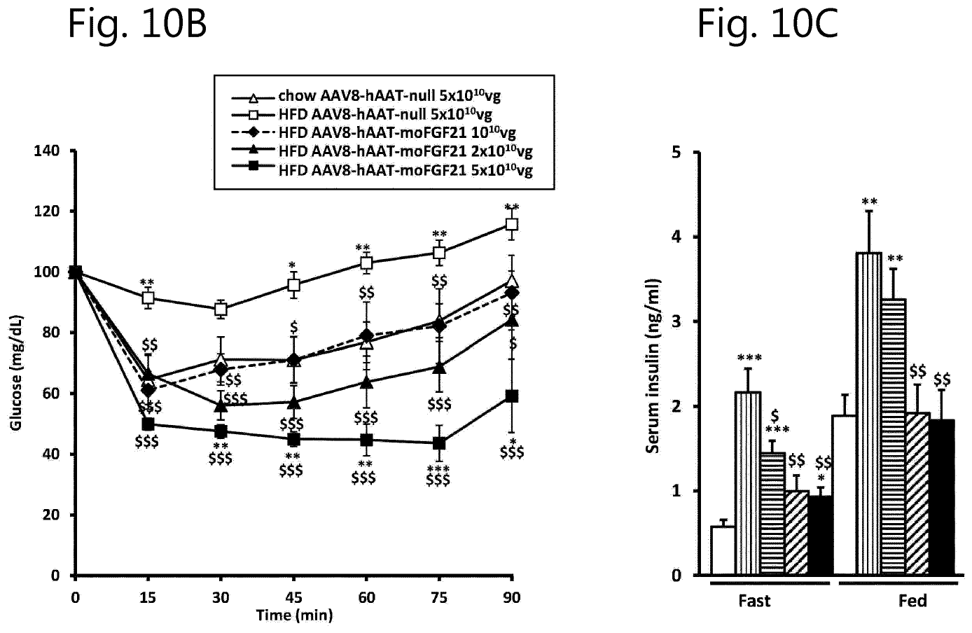

The energy expenditure of HFD-fed mice treated with 5×10¹⁰ vg of AAV8-hAAT-moFGF21 vectors during the light and dark cycles was higher than that of chow- and HFD-fed AAV8-hAAT-null mice (FIG. 10A). Animals treated with 2×10¹⁰ vg of AAV8-hAAT-moFGF21 vectors showed increased energy expenditure during the light cycle and a tendency to increase energy expenditure during the dark cycle (FIG. 10A). Animals treated with 10¹⁰ vg of AAV8-hAAT-moFGF21 vectors showed increased energy expenditure during the dark cycle (FIG. 10A). No differences were observed among chow- and HFD-fed AAV8-hAAT-null-treated animals and mice administered with 10¹⁰ vg of AAV8-hAAT-moFGF21 vectors (FIG. 10A). Altogether, these data suggest that old mice treated with AAV8-hAAT-moFGF21 have increased thermogenic activity.

Animals treated with 10¹⁰ vg or 2×10¹⁰ vg of AAV8-hAAT-moFGF21 vectors presented improved insulin sensitivity in comparison with HFD-fed mice administered with AAV8-hAAT-null vectors and their insulin sensitivity was similar to that of chow-fed mice treated with AAV8-hAAT-null vectors (FIG. 10B). Noticeably, animals administered with 5×10¹⁰ vg of AAV8-hAAT-moFGF21 vectors presented improved insulin sensitivity in comparison with chow-fed mice administered with AAV8-hAAT-null vectors (FIG. 10A). Animals treated with 10¹⁰ vg, 2×10¹⁰ vg or 5×10¹⁰ vg of AAV8-hAAT-moFGF21 vectors showed lower fasted and fed insulin circulating levels than HFD-fed AAV8-hAAT-null-treated mice (FIG. 10C). Noticeably, no differences in fed insulin circulating levels were observed between old animals administered IV with 2×10¹⁰ vg or 5×10¹⁰ vg of AAV8-hAAT-moFGF21 vectors and chow-fed AAV8-hAAT-null-treated mice (FIG. 10C).

### Example 6. Evaluation of Weight Loss by Intramuscular Administration of AAV-CMV-moFGF21 Vectors in C57B16 Mice

We also evaluated the therapeutic potential of increasing FGF21 circulating levels by the AAV-mediated genetic engineering of skeletal muscle in C57B16 mice. In order to target the skeletal muscle, the CMV promoter and the AAV1 serotype were selected. Although the CMV promoter is an ubiquitous promoter, its concomitant use together with the AAV1 capsids enables to very efficiently target the skeletal muscle without transducing the liver, as previously published (Mas et al., Diabetes 2006; Callejas et al., Diabetes 2013).

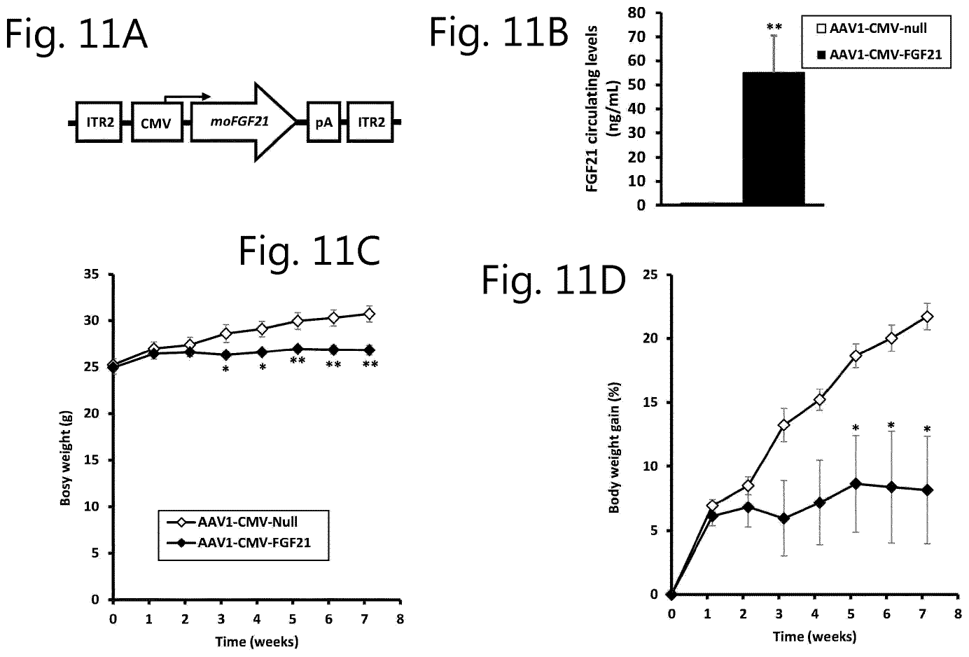

A dose of 3×10¹¹ vg of AAV1 vectors encoding a murine codon-optimized FGF21 coding sequence under the control of the ubiquitous CMV promoter (AAV1-CMV-moFGF21) (FIG. 11A) were administered by intramuscular injection in the quadriceps, gastrocnemius, and tibialis cranealis of each hind limb (5×10¹⁰ vg/muscle) of 6 to 12-week-old male C57BL6 mice. As control, age-matched C57B16 animals were administered intramuscularly in the same muscles with 3×10¹¹ vg of AAV1-CMV-null vectors (5×10¹⁰ vg/muscle). The CMV-moFGF21 construct is comprised in SEQ ID NO: 36 and the CMV-null construct is comprised in SEQ ID NO:35.

Intramuscular administration of AAV1-CMV-moFGF21 vectors mediated high secretion of FGF21 into the bloodstream (FIG. 11B). Animals treated with AAV1-CMV-moFGF21 vectors showed decreased body weight and body weight gain in comparison with AAV1-CMV-null-treated mice (FIGS. 11C and 11D).

### Example 7. Increased Protein Production by Codon-Optimized Human FGF21 Nucleotide Sequences

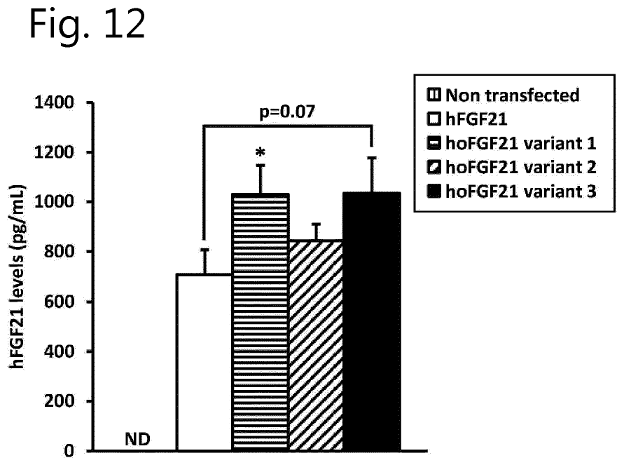

To evaluate if codon-optimization was able to mediate increased FGF21 protein production, HEK293 cells were transfected with plasmids encoding three different codon-optimized human FGF21 nucleotide sequences (SEQ ID NO's: 40-42). As control, non-transfected cells and cells transduced with wild-type hFGF21 coding sequence were used. Expression of the three codon-optimized human FGF21 sequences and the WT human FGF21 sequence was under the control of the hAAT promoter (SEQ ID NO: 47). Cells transduced with either codon-optimized human FGF21 version 1 or 3 were able to secrete higher human FGF21 levels into the culture media in comparison with wild-type or codon-optimized FGF21 variant 2 (FIG. 12), thus demonstrating increased FGF21 protein production by codon-optimization of variants 1 and 3.

### Example 8. Reversion of Obesity and Diabetes in Mice by Administration of AAV Vectors Encoding Human FGF21 (In Vivo Experiment Proving the Activity of FGF21)

HFD-fed mice are treated with AAV vectors encoding human FGF21. As controls, the same dose of AAV-null vectors is administered to chow- and HFD-fed mice.

To evaluate the capacity of human FGF21 to induce browning of WAT and thermogenic activity of BAT, to increase energy expenditure and to improve glucose and energy metabolism, the following tests are performed:
measurement of body weight and food and liquid intake weekly
measurement of body temperature
measurement of energy expenditure and respiratory quotient by indirect calorimetry
measurement of glycemia
evaluation of whole-body glucose disposal by intraperitoneal glucose tolerance test
evaluation of insulin sensitivity by intraperitoneal insulin tolerance test
analyses in tissue and serum samples, including
   examination of the level of overexpression of human FGF21 in the targeted tissue and into the bloodstream
   morphological and histological analysis.
   determination of circulating levels of hormones and cytokines
   determination of serum metabolic parameters, such as free fatty acids, glycerol, triglycerides, cholesterol and ketone bodies
   evaluation of browning capacity by examination of the presence of beige adipocytes in the inguinal fat pad by immunohistochemistry, and gene expression of classic white, brown and beige adipocyte markers

### Example 9: In Vitro Assay for Assessing FGF21 Activity

FGF21 is expected to increase glucose uptake and GLUT1 expression in 3T3-LI cells (Kharitonenkov, A. et al., 2005. J. Clin. Invest 115:1627-1635).

### Example 10. Reversion of Obesity and Improvement of Glucose Metabolism by Intra-eWAT Administration of AAV8-CAG-moFGF21-dmiRT Vectors in Ob/Ob Mice: Further Observations

We further evaluated the anti-diabetic and anti-obesogenic therapeutic potential of the AAV-mediated genetic engineering of adipose tissue with FGF21 in ob/ob mice (see Example 2).

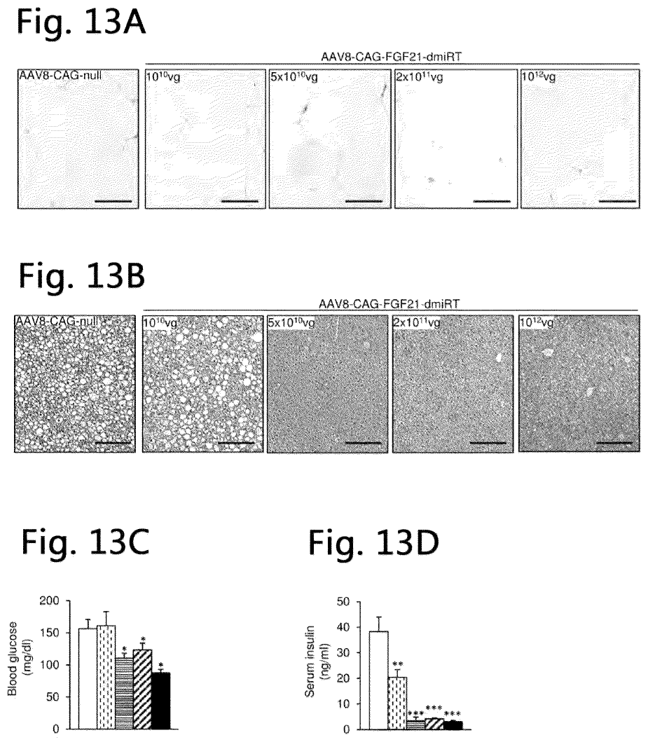

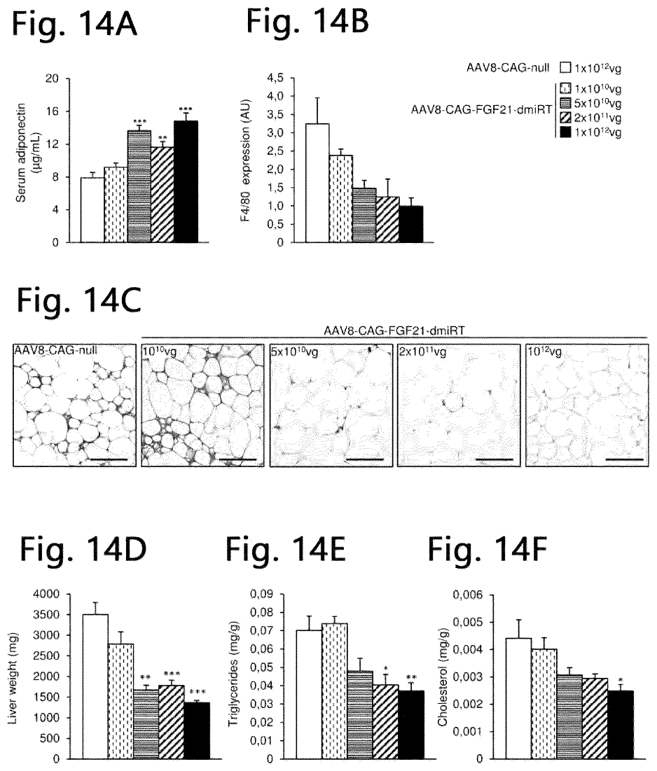

Ob/ob mice that received intra-eWAT injections of AAV8-CAG-moFGF21-dmirT vectors showed a reduction in the size of white adipocytes of the epididymal pad (FIG. 13A). Circulating adiponectin levels also increased with dose (FIG. 14A). eWAT inflammation, evaluated through Mac2 staining, was also reduced as a function of the dose of vector, as did the expression of the macrophage marker F4/80 (FIGS. 14B and C). The liver of ob/ob mice injected with null vectors or the lowest dose of AAV8-CAG-moFGF21-dmirT showed accumulation of lipid droplets in hepatocytes (FIG. 13B). The administration of doses of 5×10¹⁰ vg/mouse or higher of FGF21-encoding vectors completely prevented the development of hepatic steatosis (FIG. 13B), which correlated with the weight of the organ (FIG. 14D) and its total triglyceride and cholesterol content (FIGS. 14E and F). Further evidence that the dose of 5×10¹⁰ vg/mouse represented a threshold for therapeutic efficacy came from the analysis of glycemia and insulinemia. While the dose of 1×10¹⁰ vg/mouse did not modify the levels of blood glucose in the fed state and only partially reduced insulin levels, doses of 5×10¹⁰ vg/mouse and higher completely normalized glycemia and insulinemia (FIGS. 13C and D). Altogether, this example confirms the therapeutic potential of overexpressing FGF21 in adipose tissue.

### Example 11. Reversion of Obesity and Improvement of Glucose Metabolism by Intravenous Administration of AAV8-hAAT-moFGF21 Vectors in Ob/Ob Mice: Further Observations

We further evaluated the anti-diabetic and anti-obesogenic therapeutic potential of intravenous administration of AAV8-hAAT-moFGF21 vectors in ob/ob mice (see Example 3).

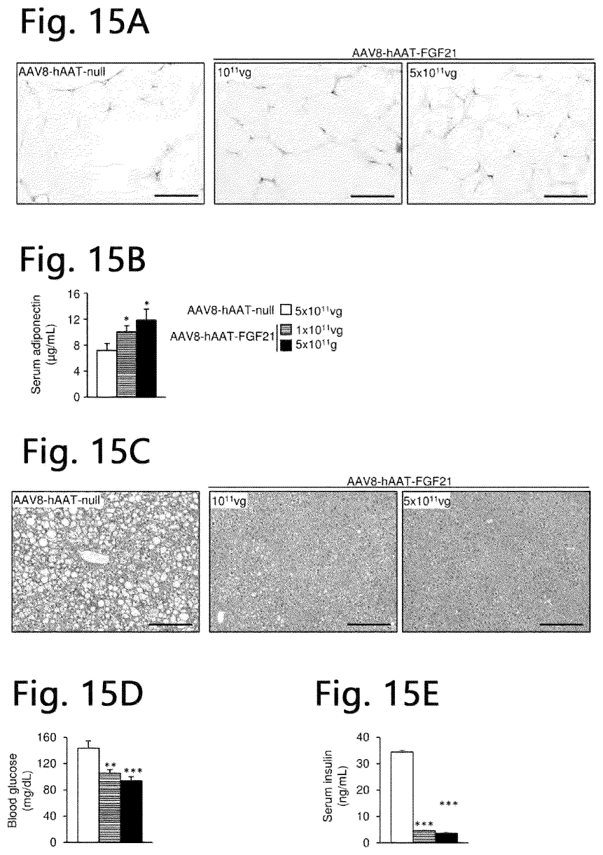

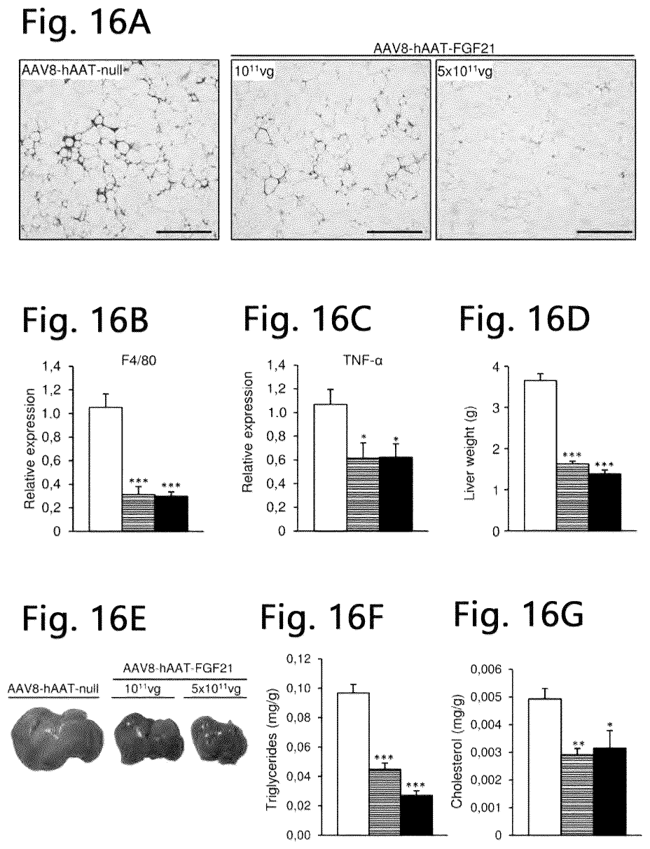

In agreement with their lower body weight, ob/ob animals overexpressing FGF21 in the liver showed significantly decreased size of white adipocytes, particularly those animals treated with 5×10¹¹ vg (FIG. 15A). This was parallel with a dose-dependent increase in circulating adiponectin levels (FIG. 15B) and decreased WAT inflammation, as evidenced by decreased staining for Mac2 and expression of F4/80 and TNF-α in eWAT (FIG. 16A-C). Noticeably, ob/ob mice treated with 5×10¹¹ vg showed a remarkable reduction in “crown-like” structures in eWAT (FIG. 16A).

While 7-month-old ob/ob mice showed marked hepatic steatosis, the liver of FGF21-treated ob/ob mice did not show accumulation of lipids in hepatocytes (FIG. 15C). This agreed with a 60% reduction in the weight of this organ (FIGS. 16D and E) as well as with a marked reduction in the total liver triglyceride and cholesterol content (FIGS. 16F and G) in ob/ob mice receiving therapeutic vectors. Ob/ob animals treated with both doses of AAV8-hAAT-moFGF21 also showed decreased fed glycemia, and their insulinemia in the fed state was reduced by ˜70% (FIGS. 15D and E).

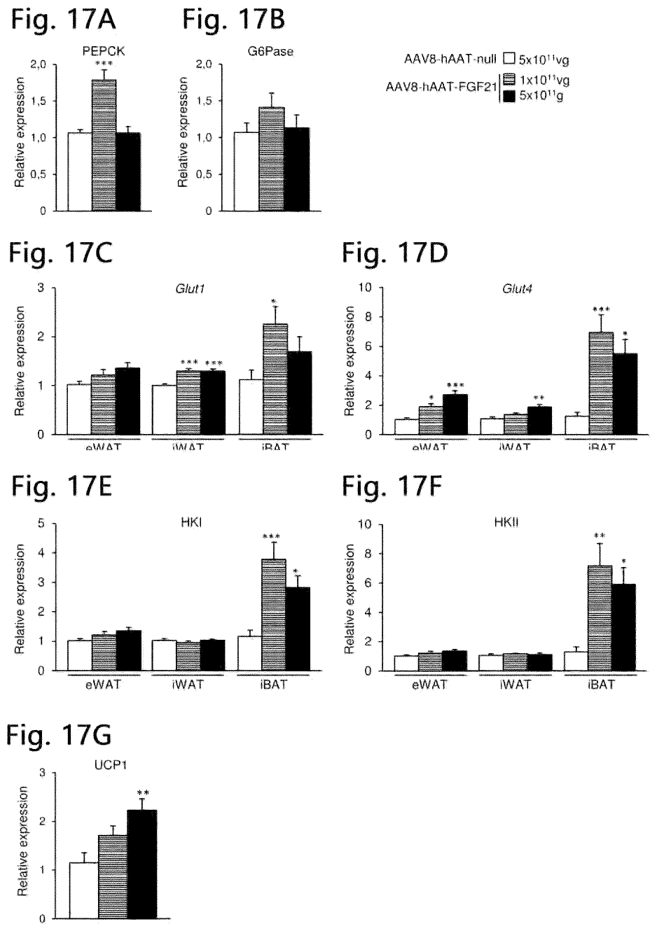

We evaluated whether the decrease in circulating glucose levels observed in ob/ob mice after AAV8-hAAT-moFGF21 treatment resulted from suppression of hepatic gluconeogenesis by measuring the expression by qPCR of phosphoenolpyruvate carboxykinase (PEPCK) and glucose-6-phosphatase (G6Pase). No changes in the expression of these enzymes were observed in the liver of AAV8-hAAT-moFGF21-treated ob/ob mice, except for the animals treated with 1×10¹¹ vg of AAV8-hAAT-moFGF21 that showed increased PEPCK expression (FIGS. 17A and B). These results suggested that AAV-mediated long-term expression of FGF21 in the liver, and the subsequent increase of circulating FGF21, did not lower glucose by inhibiting hepatic glucose production.

The glucose-lowering effects of FGF21 have also been attributed to increased glucose uptake by adipocytes and enhanced energy expenditure (Xu J. et al., 2009. AJP Endocrinol. Metab. 297: E1105-E1114; Ding X. et al., 2012. Cell Metab. 16:387-393; Camacho R. C. et al., 2013. Eur. J. Pharmacol. 715:41-45; Emanuelli B. et al., 2014. Clin. Invest. 124:515-527; Kharitonenkov A. et al., 2005. Endocrinology 148:774-781; Hondares E. et al., 2010. Cell Metab. 11:206-212; Samms R. J. et al., 2015. Cell Rep. 11:991-999). Thus, we assessed in different pads of adipose tissue (iWAT, eWAT and iBAT) the expression of key components of the glucose uptake machinery by qPCR, such as the glucose transporters Glut1 and Glut4, the glucose phosphorylating enzymes hexokinase I and II (HKI and HKI), and UCP1 in the case of iBAT. In AAV8-FGF21 treated ob/ob mice, the expression of Glut1 was increased in iWAT and iBAT (FIG. 17C), and that of Glut4 was increased in eWAT, iWAT and iBAT (FIG. 17D). HKI and HKII were upregulated only in iBAT (FIGS. 17E and F). Moreover, UCP1 expression was increased in the iBAT of ob/ob mice treated with the high dose of AAV8-hAAT-moFGF21 vectors (FIG. 17G). Altogether, these results suggest that the long-term amelioration of glycemia observed in ob/ob mice following treatment with AAV8-hAAT-moFGF21 vectors probably results from increased glucose uptake by white and brown adipocytes and enhanced thermogenesis in iBAT.

### Example 12. Long-Term Reversion of Obesity and Diabetes by Intravenous Administration of AAV8-hAAT-moFGF21 Vectors in HFD-Fed Mice and HFD-Fed Old Mice: Decreased Tissue Weight and Stable Expression Up to 1 Year

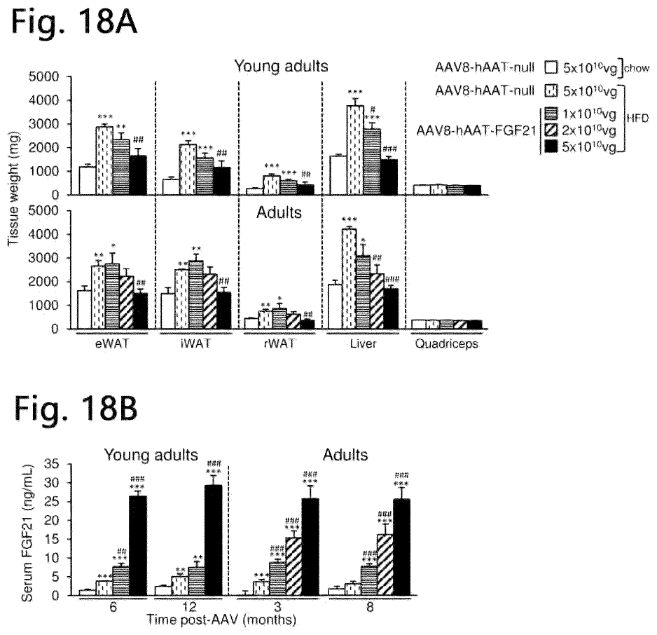

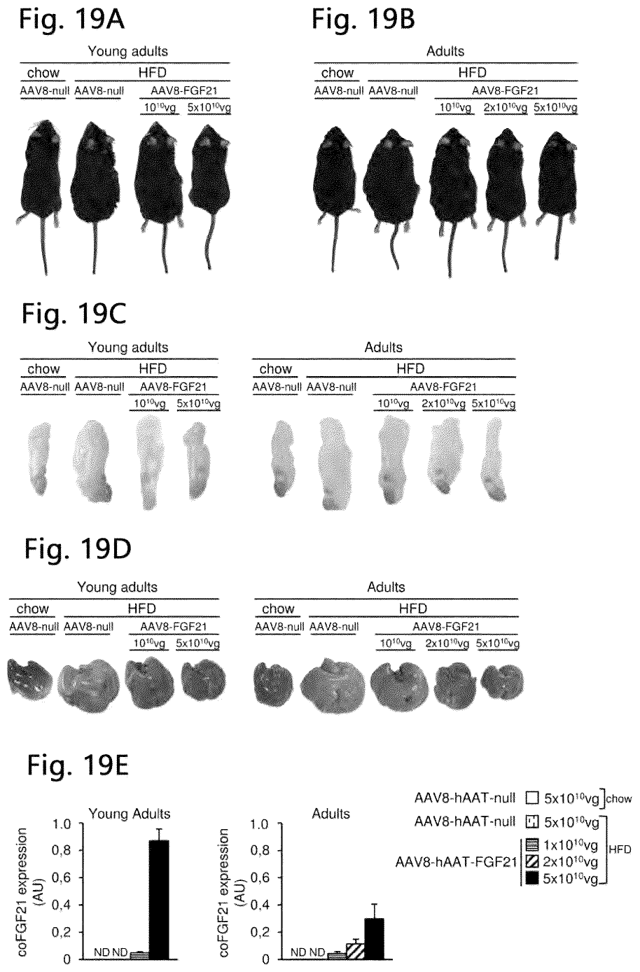

Representative images of animals belonging to all experimental groups of the studies performed in young adults or in adults (see Examples 4 and 5) are shown in FIG. 19A-B. The reversion of obesity by AAV8-hAAT-moFGF21 treatment was parallel to a dose-dependent decrease in the weight of the main white adipose tissue (WAT) depots, such as the epididymal (eWAT), inguinal (iWAT) and retroperitoneal (rWAT) fat pads, both in animals treated as young adults or as adults (FIG. 18A and FIG. 19C). The HFD-induced increase in the weight of the liver was completely normalized by FGF21 gene transfer at the highest doses of vector used, whereas the weight of the quadriceps was unchanged by the diet or AAV delivery (FIG. 18A and FIG. 19D). AAV8-hAAT-moFGF21-treated mice of both ages showed specific overexpression of codon-optimized FGF21 in the liver (FIG. 19E), which resulted in secretion of FGF21 into the bloodstream in a dose-dependent manner in both groups of mice, with levels remaining stable for up to 1 year after a single administration of the vector (FIG. 18B).

### Example 13. Long-Term Reversion of Obesity and Diabetes by Intravenous Administration of AAV8-hAAT-moFGF21 Vectors in HFD-Fed Mice and HFD-Fed Old Mice: Increased Locomotor Activity and Investigating the Thermogenic Mechanism

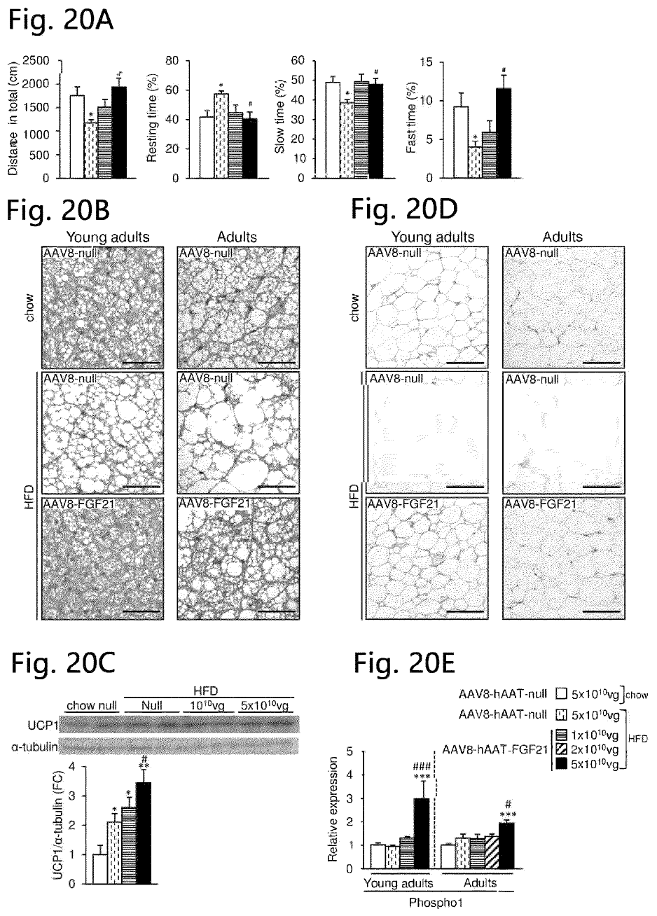

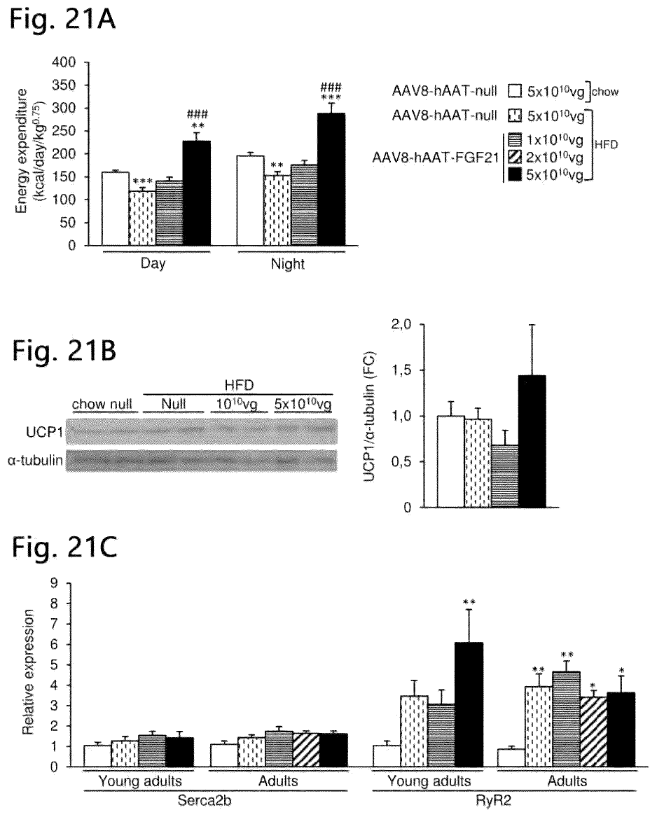

Increased energy expenditure (see Examples 4 and 5) was also seen in animals treated as young adults 10 months after AAV8-hAAT-moFGF21 delivery (FIG. 21A). This observation was in agreement with AAV8-hAAT-moFGF21-mediated effects on locomotor activity. In contrast to the hypoactivity observed in the open field test in the animals fed a HFD that received AAV8-null vectors, mice treated with 5×10¹⁰ vg AAV8-hAAT-moFGF21 as young adults showed the same degree of spontaneous locomotor activity than chow-fed, null-injected animals. As shown in FIG. 20A, 1 year after AAV8-hAAT-moFGF21 delivery, treated animals travelled more distance, rested less time, and spent more time doing slow and fast movements than untreated HFD-fed controls. Given that changes in energy expenditure may reflect changes in thermogenesis, we evaluated the degree of activation of the brown adipose tissue (BAT). Both mice treated as young adults or adults with 5×10¹⁰ vg AAV8-hAAT-moFGF21 showed decreased lipid deposition in iBAT (FIG. 20B). The content of UCP1 protein in BAT was increased in a dose-dependent manner in mice treated with AAV8-hAAT-moFGF21 vectors as young adults (FIG. 20C), consistent with an increase in non-shivering thermogenesis induced by FGF21 gene transfer to the liver.

The browning of the subcutaneous WAT, characterized by the appearance of beige adipocytes, is also associated with increases in energy expenditure (Harms & Scale, 2013). To evaluate if browning was accountable for the enhancement of energy expenditure observed following AAV8-hAAT-moFGF21 treatment, histological evaluation of iWAT was performed. In agreement with the decreased weight of this pad (FIG. 18A), the adipocytes of HFD-fed AAV8-hAAT-moFGF21-treated animals were smaller than those of HFD-fed null-injected animals (FIG. 20D). Treatment with AAV8-hAAT-moFGF21 vectors, nevertheless, did not result in increased detection of multilocular beige adipocytes in iWAT at any of the doses tested, either in animals treated as young adults or adults (FIG. 20D). Accordingly, there were no statistically significant differences in the levels of UCP1 protein in iWAT between the HFD-fed groups (FIG. 21B).

The creatine-driven substrate cycle and sarco/endoplasmic reticulum Ca2+-ATPase 2b (Serca2b)-mediated calcium cycling can increase thermogenesis in iWAT independently of UCP1 (Kazak L. et al., 2015. Cell 163:643-655; Ikeda K. et al., 2017. Nat. Med. 23:1454-1465). Higher levels of expression of phosphatase orphan 1 (Phospho1), an enzyme involved in the creatine-driven substrate cycle, were observed in iWAT of HFD-fed mice treated with 5×10¹⁰ vg of AAV8-hAAT-moFGF21 when compared with age-matched, chow- and HFD-fed control groups (FIG. 20E), suggesting that the activity of the creatine-driven cycle was probably increased as a result of FGF21 gene transfer. Regarding the calcium cycling-dependent thermogenic mechanism, no differences in the expression levels of Serca2b were detected in the iWAT of animals treated with AAV8-hAAT-moFGF21 vectors when compared with chow- or HFD-fed null-treated animals (FIG. 21C). On the other hand, the iWAT expression of ryanodine receptor 2 (RyR2), another enzyme involved in the same cycle, was increased by HFD-feeding in both null- and AAV8-hAAT-moFGF21-treated mice (FIG. 21C). Altogether, these results suggest that the calcium cycling-dependent thermogenic mechanism is not involved in the improvement of whole-body energy homeostasis observed after AAV-FGF21 treatment.

### Example 14. Long-Term Reversion of Obesity and Diabetes by Intravenous Administration of AAV8-hAAT-moFGF21 Vectors in HFD-Fed Mice and HFD-Fed Old Mice: Glucagon Levels, Islet Hyperplasia and Glucose Tolerance

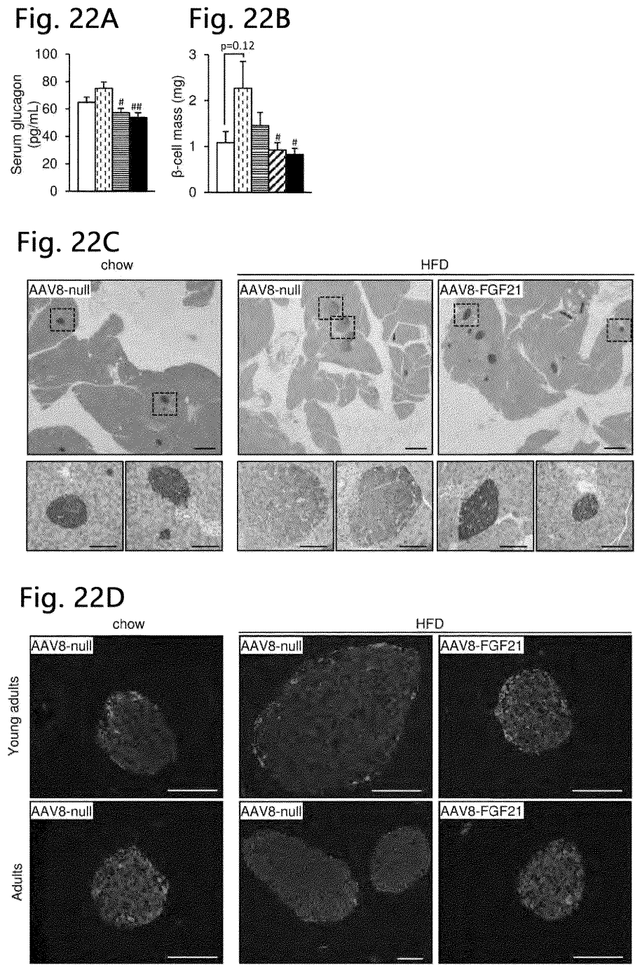

Moreover, HFD-fed animals treated as young adults with AAV8-hAAT-moFGF21 vectors showed decreased circulating levels of glucagon compared with HFD-fed null-treated mice (FIG. 22A).

While AAV8-null-treated mice developed islet hyperplasia as a consequence of HFD feeding, the β-cell mass of animals treated with AAV8-hAAT-FGF21 vectors (at the doses of 2×10¹⁰ or 5×10¹⁰ vg/mouse) was similar to that of control mice fed a chow diet (FIGS. 22B and C). Double immunostaining for insulin and glucagon of pancreatic sections from HFD-fed AAV8-hAAT-moFGF21-treated mice showed normal distribution of a and β cells in the islets of these animals, with localization of glucagon-expressing cells in the periphery of the islet and of insulin-expressing cells in the core (FIG. 22D).

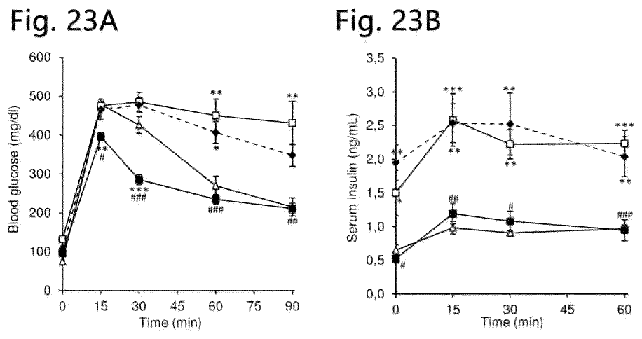

To evaluate glucose tolerance in FGF21-treated mice, an intraperitoneal glucose tolerance test (GTT) (2 g glucose/kg bw) was performed 10 weeks after AAV administration. HFD-fed animals injected with either null or FGF21-encoding vectors at a dose of 1×10¹⁰ vg/mouse were glucose intolerant and showed markedly increased circulating levels of insulin during the GTT (FIGS. 23A and B). In contrast, animals treated with 5×10¹⁰ vg/mouse of AAV8-hAAT-moFGF21 showed improved glucose clearance when compared to chow-fed control mice (FIG. 23A). Insulin levels were indistinguishable between these two experimental groups (FIG. 23B). These results further confirmed improved insulin sensitivity in HFD-fed mice treated with 5×10¹⁰ vg/mouse of AAV8-hAAT-moFGF21.

### Example 15. Reversion of HFD-Associated WAT Hypertrophy and Inflammation by Intravenous Administration of AAV8-hAAT-moFGF21 Vectors

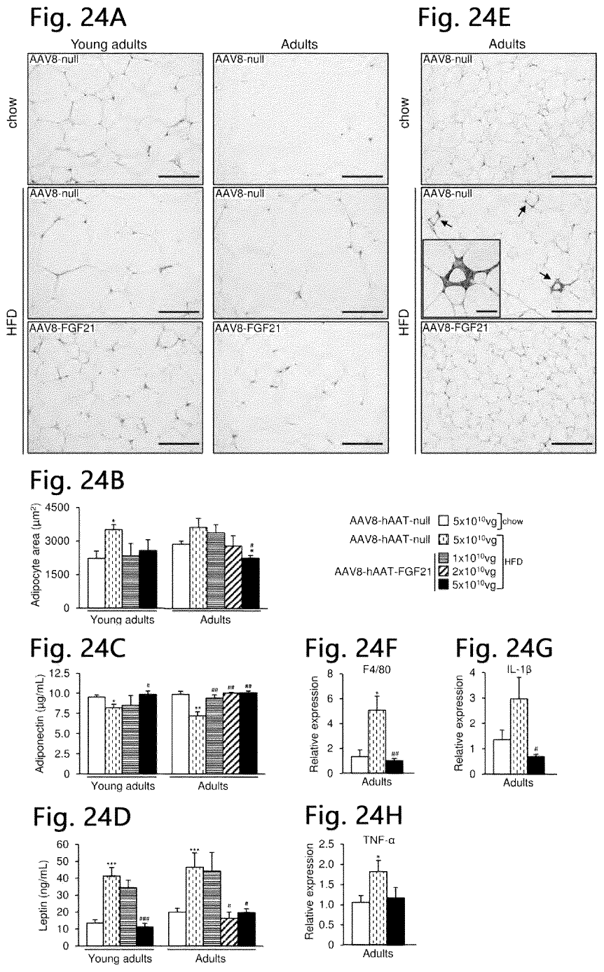

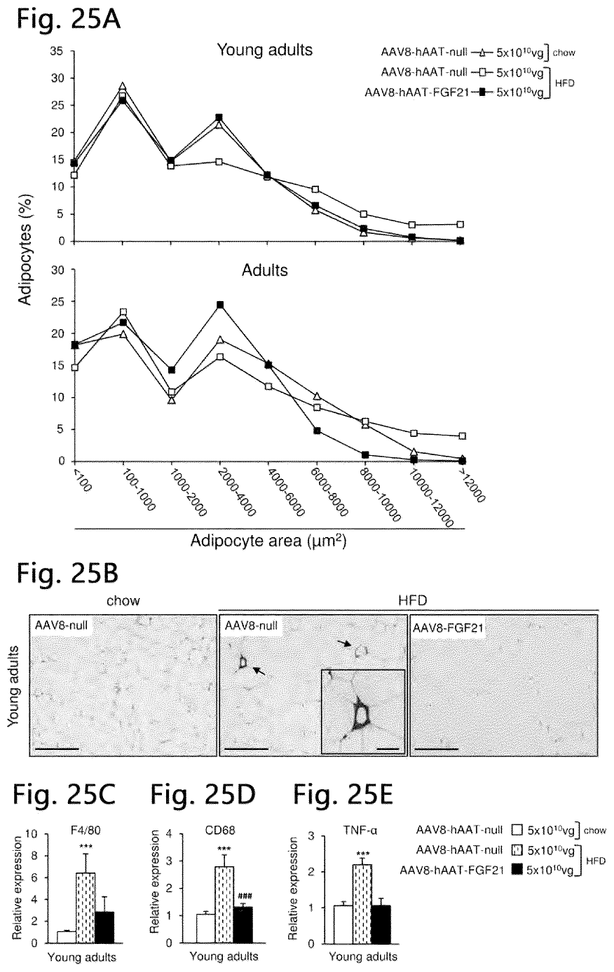

HFD-feeding induces an increase in the size of WAT adipocytes (Sattar N. & Gill J. M. R., 2014. BMC Med. 12:123). Administration of FGF21-encoding vectors counteracted this increase (FIG. 24A). Morphometric analysis of WAT revealed that the area of white adipocytes of animals treated as young adults with 1×10¹⁰ or 5×10¹⁰ vg of vector, and of mice treated as adults with 2×10¹⁰ or 5×10¹⁰ vg of vector was similar to that of animals fed a chow diet (FIG. 24B). In both groups of FGF21-treated animals, there was a redistribution of the size of adipocytes, with a greater proportion of smaller adipocytes (FIG. 25A). In agreement with the decrease in adiposity and reversal of WAT hypertrophy, adiponectin and leptin levels were also normalized in animals treated with highest doses of AAV8-hAAT-moFGF21 vectors, irrespective of the age of initiation of the treatment (FIGS. 24C and D).

Obesity also causes the inflammation of WAT (Hajer G. R. et al., 2008. Eur. Heart J. 29:2959-2571). Thus, we analyzed inflammation in this tissue through immunostaining for the macrophage-specific marker Mac2 and the expression of pro-inflammatory molecules. While HFD-fed mice showed increased presence of macrophages, revealed as “crown-like” structures, in the eWAT, animals treated as young adults or adults with 5×10¹⁰ vg AAV8-hAAT-moFGF21 had no sign of macrophage infiltration (FIG. 24E and FIG. 25B). This was parallel to the normalization in the expression of the macrophage markers F480 and CD68 and of the pro-inflammatory cytokines TNFα and IL-1β (FIG. 24F-H and FIG. 25C-E), indicating that FGF21 expression counteracted the inflammation of WAT associated to obesity.

### Example 16. Reversal of Hepatic Steatosis, Inflammation and Fibrosis by Intravenous Administration of AAV8-hAAT-moFGF21 Vectors

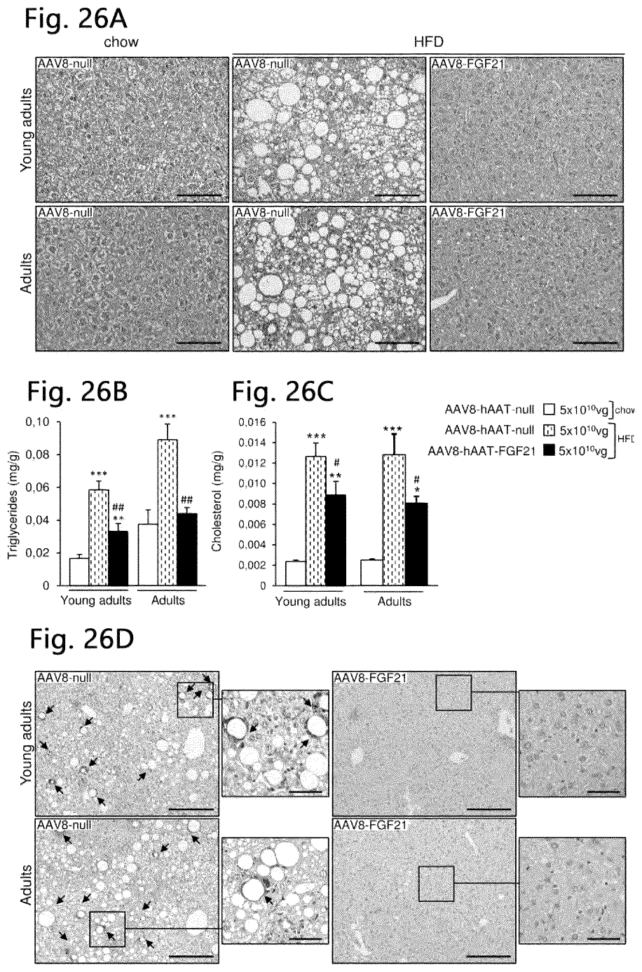

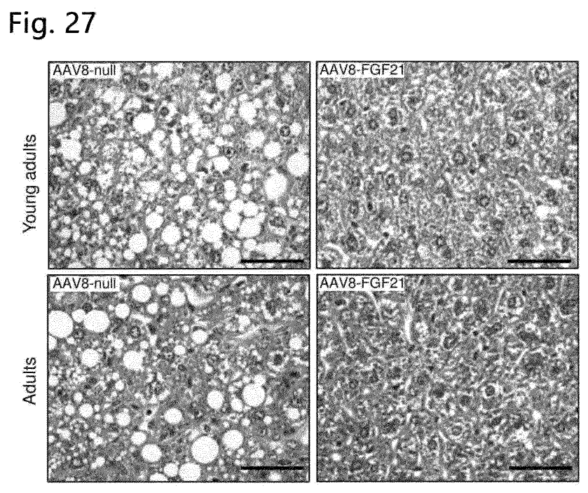

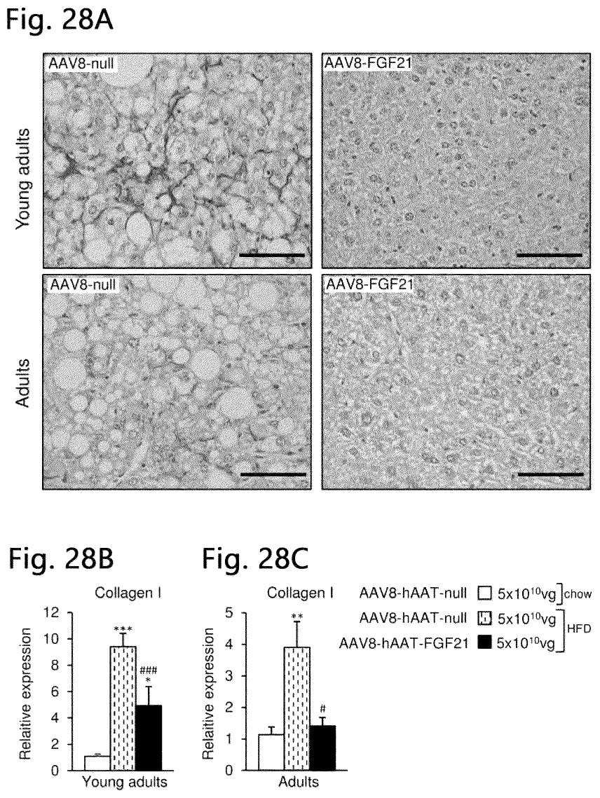

Histological analysis of the liver showed that all null-treated animals fed a HFD had marked hepatic steatosis at the time of sacrifice (FIG. 26A-D). In contrast, HFD-fed mice receiving 5×10¹⁰ vg AAV8-hAAT-moFGF21 as young adults or as adults evidenced reversal of this pathological deposition of lipids (FIG. 26A). These histological findings were parallel to a marked reduction in the total liver triglyceride and cholesterol content of 5×10¹⁰ vg AAV8-hAAT-moFGF21-treated animals (FIGS. 26B and C). In addition, animals fed a HFD and treated with 5×10¹⁰ vg AAV8-hAAT-moFGF21 vectors when young adults or adults showed no sign of hepatic inflammation, as evidenced by the lack of staining for Mac2, which revealed increased presence of macrophages in the livers of null-treated HFD-fed mice (FIG. 26D). Finally, FGF21 gene transfer to the liver reversed hepatic fibrosis. While collagen fibers were readily detectable following PicroSirius Red staining or Masson's trichrome staining of liver sections from animals fed a HFD and injected with control null vectors, they were undetectable in the livers of AAV8-hAAT-moFGF21 treated mice (FIG. 28A and FIG. 27). These mice also showed markedly reduced hepatic expression of collagen 1 (FIGS. 28B and C). Altogether these findings indicated that AAV8-hAAT-moFGF21 treatment protected from the development of HFD-induced non-alcoholic steatohepatitis (NASH).

### Example 17. Long-Term Safety of Liver-Directed AAV-FGF21 Treatment

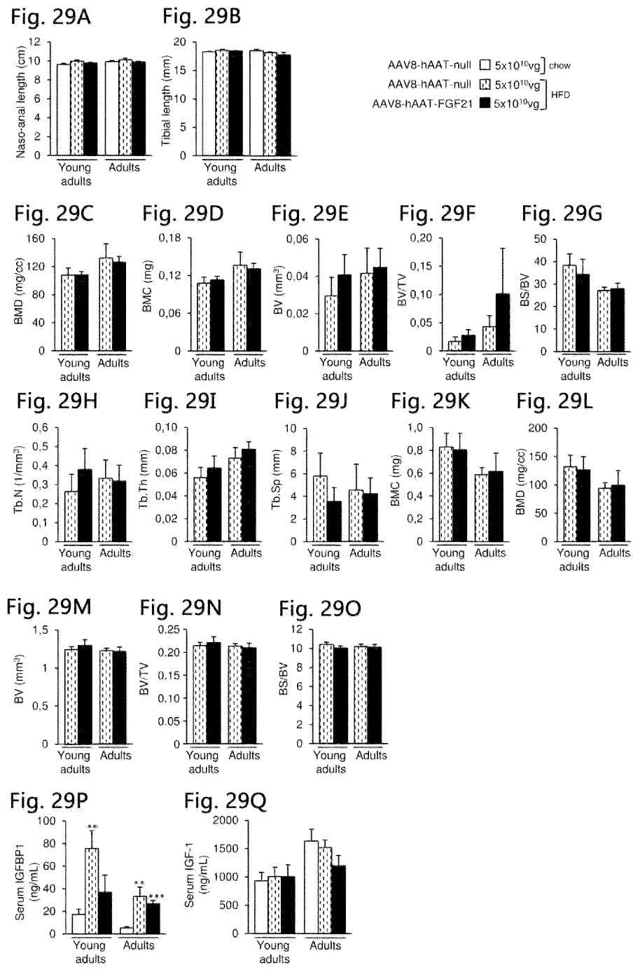

Pharmacological treatment with FGF21 or transgenic overexpression have been associated with perturbation of bone homeostasis through increased bone resorption, which could cause bone loss (Wei W. et al., 2012. Proc. Natl. Acad. Sci. 109:3143-3148; Wang X. et al., 2015. Cell Metab. 22:811-824; Charoenphandhu N. et al., 2017. J. Bone Miner. Metab. 35:142-149; Talukdar S. et al., 2016. Cell Metab. 23:427-440; Kim A. M. et al., 2017. Diabetes, Obes. Metab). Given the therapeutic potential of AAV8-hAAT-moFGF21 for the treatment of obesity and diabetes, we evaluated the long-term effects of gene transfer on the bones of the animals treated with the highest dose of vector. At the time of sacrifice (˜16.5 months of age), the naso-anal length and the tibial length were normal in the animals that were administered with AAV8-hAAT-moFGF21 vectors at 9 or 29 weeks of age (FIGS. 29A and B). We then examined bone structure by micro-computed tomography (μCT). Analysis of the proximal epiphysis of the tibia revealed no significant differences in the trabecular and cortical bone of mice fed a HFD and administered with 5×10¹⁰ vg AAV8-hAAT-moFGF21 in comparison with age-matched mice treated with null vectors. Specifically, no differences were documented in the bone mineral density (BMD) (FIG. 29C), bone mineral content (BMC) (FIG. 29D), bone volume (BV) (FIG. 29E), bone volume/tissue volume ratio (BV/TV) (FIG. 29F), bone surface/bone volume ratio (BS/BV) (FIG. 29G), trabecular number (Tb.N) (FIG. 29H), trabecular thickness (Tb.Th) (FIG. 291) or trabecular separation (Tb.Sp) (FIG. 29J). Similarly, the analysis of the compact bone at the tibial diaphysis showed no differences in the BMC, BMD, BV, BV/TV or BS/BV between the HFD-fed null-injected or FGF21-treated groups (FIG. 29K-O).

The pathological effects of FGF21 have been reported to be mediated, at least in part, by increased production of Insulin-like Growth Factor Binding Protein 1 (IGFPB1) by the liver (Wang X. et al., 2015. Cell Metab. 22:811-824). In agreement with the lack of bone alterations, high-dose AAV8-hAAT-moFGF21 treatment did not lead to an increase in the levels of circulating IGFBP1 protein in animals treated 12 (young adults) or 6 (adults) months earlier when compared to null-injected HFD-fed mice (FIG. 29P). Circulating IGF1 levels were also normal in all experimental groups (FIG. 29Q). Altogether these results support the safety for bone tissue of AAV-mediated FGF21 gene transfer to the liver.

### Example 18. Prevention of HFD-Induced Liver Tumours by Intravenous Administration of AAV8-hAAT-moFGF21 Vectors

Long-term feeding (>60 weeks) with a HFD has been associated with increased incidence of liver neoplasms in C57BL/6J mice (Hill-Baskin A.E. et al., 2009. Hum. Mol. Genet. 18:2975-2988; Nakagawa H., 2015. World J. Hepatol. 7:2110). In our study in animals that initiated the HFD as young adults and maintained it for 60 weeks we found liver tumours in 66.7% (6/9) of animals injected with null-vectors. Animals treated with AAV8-hAAT-moFGF21 vectors were protected from HFD-induced development of liver neoplasms: 0% (0/8) of animals treated with the 5×10¹⁰ vg of FGF21-encoding vectors showed tumours, and the incidence was 40% (4/10) in the cohort treated with the lowest dose (1×10¹⁰ vg). None (0/11) of the chow-fed mice developed tumours in the same period of time (Table 1).

TABLE 1
Liver tumour incidence in young adults.

| Group | Hepatocarcinoma | Hepatocarcinoma (%) |
|---|---|---|
| Chow AAV8-null | 0/11 | 0% |
| HFD AAV8-null | 6/9 | 66.7% |
| HFD AAV8-FGF21 (1 × | 4/10 | 40% |
| 10¹⁰ vg/mouse) | | |
| HFD AAV8-FGF21 (5 × | 0/8 | 0% |
| 10¹⁰ vg/mouse) | | |

### Example 19. Amelioration of STZ-Induced Hyperglycemia by Liver-Specific AAV8-Mediated FGF21 Overexpression

### Material and Methods

### Animals

We used 9-week-old male C57b16 mice. Mice had free access to a standard diet and were kept under a 12 h light-dark cycle (lights on at 08:00 hours). For diabetes induction, mice received five intraperitoneal injections, on consecutive days, of streptozotocin (50 mg/kg) dissolved in 0.1 mol/l citrate buffer (pH 4.5). Blood glucose levels were assessed using an analyser (Glucometer Elite; Bayer, Leverkusen, Germany). Animal care and experimental procedures were approved by the Ethics Committee in Animal and Human Experimentation of the Universitat Autónoma de Barcelona.

### In Vivo Administration of AAV Vectors

For systemic administration, AAV vectors were diluted in 200 μl of 0.001% F68 Pluronic® (Gibco) in PBS and injected via the tail vein.

### Results

In order to test the protective potential against type 1 diabetes of AAV-derived FGF21, 5×10¹⁰ vg or 2×10¹¹ vg of AAV8 vectors encoding a codon-optimized murine FGF21 coding sequence under the control of the hAAT promoter (AAV8-hAAT-moFGF21) were administered IV to male 9-week-old C57B16 mice. Control mice received 2×10¹¹ vg of AAV8-hAAT-Null vectors. Two weeks post-AAV administration, all animals were treated with streptozotocin (STZ) (5 doses of 50 mg/kg; 1 dose per day) to trigger the diabetic process.

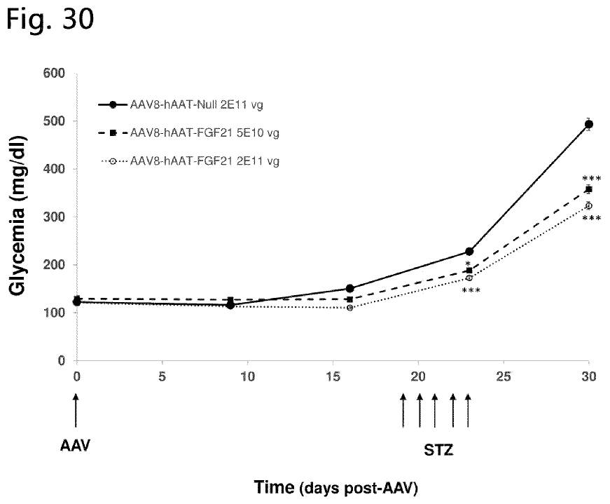

Analysis of the blood glucose levels revealed that animals treated with AAV8 vectors encoding moFGF21 displayed lower circulating glucose levels than C57B16 mice treated with AAV8-hAAT-Null vectors (FIG. 30).

### Example 20. Extension of Healthy Lifespan by Intramuscular Administration of AAV-CMV-moFGF21 Vectors in C57B16 Mice Due to the Prevention of Weight Gain and Insulin Resistance Associated with Aging

Skeletal muscle (Skm) is a readily accessible tissue and has been used to produce secretable therapeutic proteins (Haurigot V. et al., 2010. J. Clin. Invest. 123:3254-3271; Callejas D. et al., 2013. Diabetes 62:1718-1729; Jaén M. L. et al., 2017. Mol. Ther. Methods Clin. Dev. 6:1-7). To explore if the Skm could represent a viable source of circulating FGF21, AAV vectors of serotype 1, which show a high tropism for Skm (Chao L. et al., 2000. J. Clin. Invest. 106:1221-1228; Wu Z. et al., 2006. J. Virol. 80:9093-9103; Lisowski L. et al., 2015. Curr. Opin. Pharmacol. 24:59-67), carrying murine optimized FGF21 under the control of the CMV promoter were used (AAV1-CMV-moFGF21). Vectors were injected at a dose of 5×10¹⁰ vg/muscle to the quadriceps, gastrocnemius and tibialis cranialis of both legs (total dose, 3×10¹¹ vg/mouse) of 8-week-old C57B16 mice. Control animals were injected with AAV1-CMV-Null vectors at the same dose. The use of healthy mice fed a standard diet further allowed us to evaluate the long-term safety of FGF21 gene therapy.

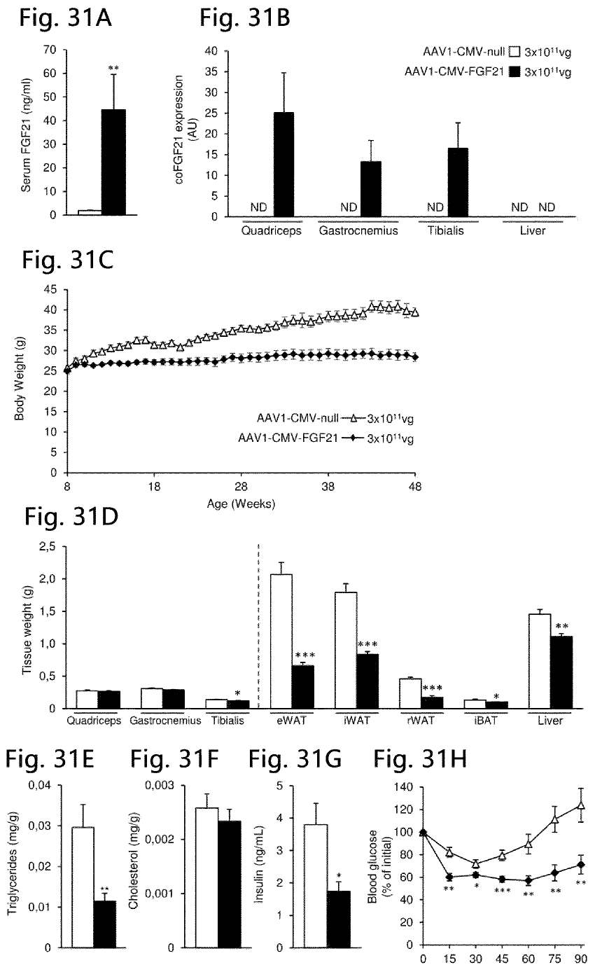

Eleven-month-old animals injected with FGF21-encoding vectors at 8 weeks of age showed a marked increase in circulating FGF21 (FIG. 31A), which was parallel to high levels of expression of vector-derived FGF21 in the 3 injected muscles (FIG. 31B). In agreement with previous reports, this combination of vector serotype, promoter and route of administration did not lead to expression of the transgene in the liver (FIG. 31B). At the end of the ˜10-month follow-up period, mice injected intramuscularly with AAV1-CMV-moFGF21 maintained the body weight they had at the initiation of the study and were ˜38% slimmer than controls, which steady increased their weight as animals aged (FIG. 31C). While the weight of the muscles was barely affected by FGF21 gene transfer, the weight of the white and brown depots as well as the liver were considerably reduced (FIG. 31D). Indeed, the weight of the WAT pads analysed was reduced by >50% (FIG. 31D). Moreover, mice treated with AAV1-CMV-moFGF21 showed a marked reduction in the hepatic total triglyceride content (FIG. 31E). No changes in hepatic cholesterol levels were observed (FIG. 31F). As opposed to null-injected animals, animals treated with AAV1-CMV-moFGF21 showed normoglycemia (data not shown) and reduced insulinemia when they were approximately 1-year-old (FIG. 31G). Accordingly, FGF21-treated mice showed markedly improved insulin sensitivity at the end of the study (FIG. 31H). Altogether, this study demonstrates that administration of AAV vectors that leads to therapeutically-relevant levels of circulating FGF21 is safe in the long-term in healthy, and may be used to reverse the increase in body weight and insulin resistance associated to aging.

### Example 21. Reversion of Obesity and Diabetes by Intramuscular Administration of AAV1-CMV-moFGF21 Vectors in HFD-Fed C57B16 Mice

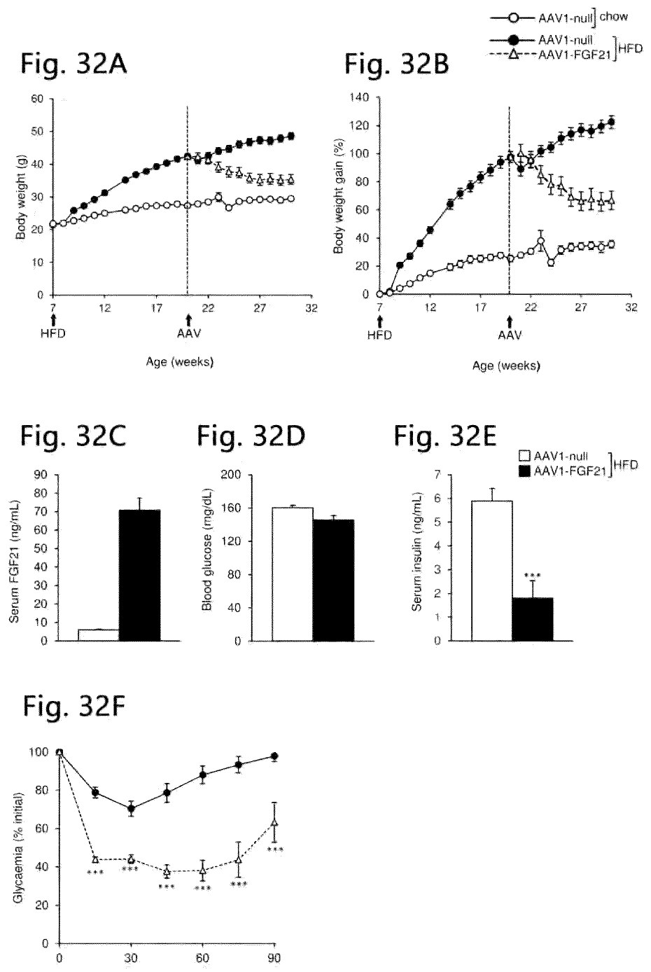

We next evaluated whether im administration of AAV1-CMV-moFGF21 vectors was also able to reverse obesity and insulin resistance. To this end, two-month-old C57B16 mice were fed either a chow or a HFD for 12 weeks. During these first 3 months of follow-up, while the weight of chow-fed animals increased by 20%, animals fed a HFD became obese (95% body weight gain) (FIGS. 32A and B). Vectors were then injected at a dose of 5×10¹⁰ vg/muscle to the quadriceps, gastrocnemius and tibialis cranialis of both legs (total dose, 3×10¹¹ vg/mouse) of obese C57B16 mice. As controls, another cohort of obese mice and the cohort of chow-fed mice received 3×10¹¹ vg of non-coding null vectors (AAV1-CMV-null). Following AAV delivery, mice were maintained on chow or HFD feeding. Animals treated with AAV1-CMV-moFGF21 experienced progressive loss of body weight (FIGS. 32A and B). The reversion of obesity by AAV1-CMV-FGF21 treatment was parallel to an increase in the circulating levels of FGF21 (FIG. 32C). Null-treated mice fed a HFD showed normal fed glycemia (FIG. 32D), but were hyperinsulinemic (FIG. 32E), suggesting that these mice had developed insulin resistance. In contrast, HFD-fed mice treated with AAV1-CMV-moFGF21 were, by the end of the study, normoglycemic and normoinsulinemic (FIGS. 32D and E). Moreover, animals administered with AAV1-CMV-moFGF21 showed greater insulin sensitivity than their HFD-fed controls (FIG. 32F).

### Example 22. Increased FGF21 Circulating Levels by Codon-Optimized Human FGF21 Nucleotide Sequences

To evaluate if codon-optimization was able to mediate increased FGF21 circulating levels, 8-week-old male C57B16 mice were hydrodynamically injected with plasmids encoding three different codon-optimized human FGF21 nucleotide sequences (SEQ ID NO's: 40-42) under the control of the hAAT promoter. As control, non-treated mice and mice hydrodynamically injected with a plasmid encoding wild-type hFGF21 coding sequence under the control of the hAAT promoter were used.

### Material and Methods

In vivo delivery of plasmids into mice by hydrodynamic tail vein injection Plasmid DNA was diluted in saline in a volume (ml) equal to ˜10% of the animals' average body weight (grams) and was manually injected into the lateral tail vein in less tan 5 seconds. Before the injection, the animals were put under a 250W infrared heat lamp (Philips) for a few minutes to dilate the blood vessels and facilitate viewing and easier access to the tail vein. A plastic restrainer (Harvard Apparatus) was used to secure the animal for injection. No anaesthesia was used as it is not necessary so long as an appropriate restraining device is employed. We used 26G 3/8 in. gauge hypodermic needles (BD), the largest feasible needle gauge that fit snugly into the access vein, to inject the animals.

### Results

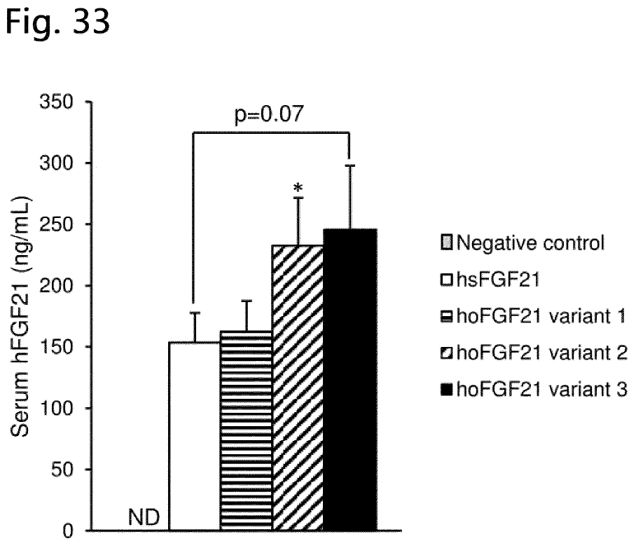

Mice treated with either codon-optimized human FGF21 version 2 or 3 were able to secrete higher human FGF21 levels into the circulation in comparison with wild-type or codon-optimized FGF21 variant 1 (FIG. 33, thus demonstrating increased FGF21 protein production by codon-optimization of variants 2 and 3.

### Example 23. In Vitro and In Vivo Increased FGF21 Expression and Protein Production Levels by hAAT-moFGF21, CAG-moFGF21-doublemiRT and CMV-moFGF21 Expression Cassettes

### Material and Methods

In vivo delivery of plasmids into mice by hydrodynamic tail vein injection Plasmid DNA was diluted in saline in a volume (ml) equal to ˜10% of the animals' average body weight (grams) and was manually injected into the lateral tail vein in less tan 5 seconds. Before the injection, the animals were put under a 250W infrared heat lamp (Philips) for a few minutes to dilate the blood vessels and facilitate viewing and easier access to the tail vein. A plastic restrainer (Harvard Apparatus) was used to secure the animal for injection. No anaesthesia was used as it is not necessary so long as an appropriate restraining device is employed. We used 26G 3/8 in. gauge hypodermic needles (BD), the largest feasible needle gauge that fit snugly into the access vein, to inject the animals.

### Results

In vitro

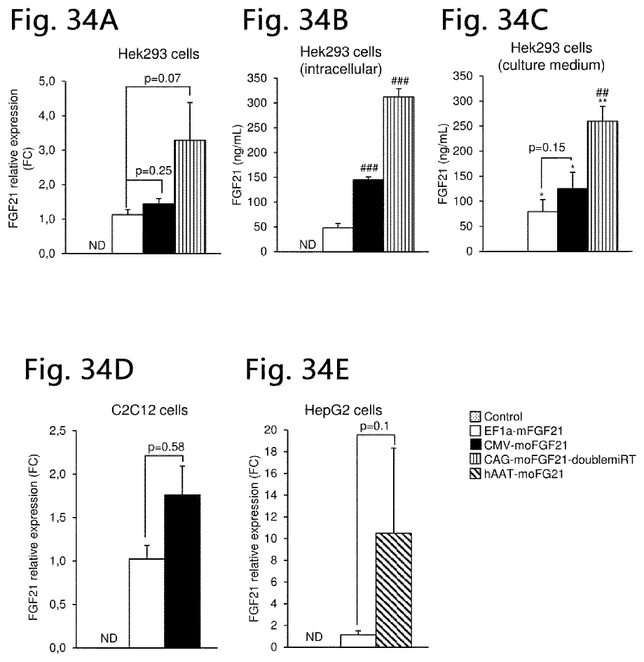

HEK293 cells were transfected with plasmids encoding the WT murine FGF21 coding sequence under the control of the elongation factor 1a (EF1a) promoter (EF1a-mFGF21) (Zhang et al., EBioMedicine 15 (2017) 173-183) (SEQ ID NO:57) or a codon-optimized murine FGF21 coding sequence under the control of the CMV promoter (CMV-moFGF21) or the CAG promoter in conjunction with four tandem repeats of the miRT122a sequence and four tandems repeats of the miRT1 sequence (CAG-moFGF21-doublemiRT). As control, non-transfected cells were used. HEK293 cells transduced with CAG-moFGF21-doublemiRT expressed higher levels of FGF21 in comparison with cells transduced with EF1a-mFGF21 or non-transduced cells (FIG. 34A). Moreover, HEK293 cells transduced with CAG-moFGF21-doublemiRT also showed higher intracellular FGF21 protein content and higher FGF21 protein levels in the culture medium (FIGS. 34B and C). Although HEK293 cells transduced with EF1a-mFGF21 or CMV-moFGF21 expressed similar levels of FGF21 (FIG. 34A), HEK293 cells transduced with CMV-moFGF21 showed higher intracellular FGF21 protein content and higher FGF21 protein levels in the culture medium (FIGS. 34B and C).

C2C12 cells were transfected with plasmids encoding the WT murine FGF21 coding sequence under the control of the EF1a promoter (EF1a-mFGF21) (Zhang et al., EBioMedicine 15 (2017) 173-183) or a codon-optimized murine FGF21 coding sequence under the control of the CMV promoter (CMV-moFGF21). As control, non-transfected cells were used. C2C12 cells transduced with CMV-moFGF21 expressed higher levels of FGF21 in comparison with cells transduced with EF1a-mFGF21 or non-transduced cells (FIG. 34D).

HepG2 cells were transfected with plasmids encoding the WT murine FGF21 coding sequence under the control of the EF1a promoter (EF1a-mFGF21) (Zhang et al., EBioMedicine 15 (2017) 173-183) or a codon-optimized murine FGF21 coding sequence under the control of the hAAT promoter (hAAT-moFGF21). As control, non-transfected cells were used. HepG2 cells transduced with hAAT-moFGF21 expressed higher levels of FGF21 in comparison with cells transduced with EF1a-mFGF21 or non-transduced cells (FIG. 34E).

### In Vivo

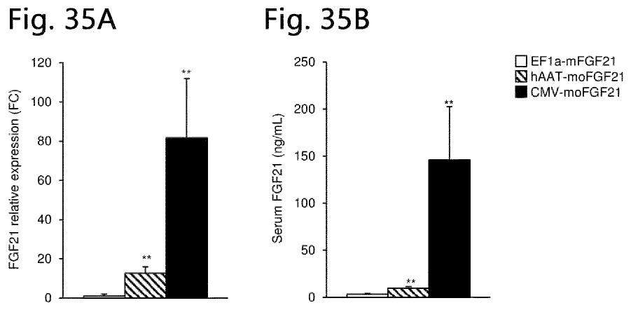

8-week-old male C57B16 mice were hydrodynamically administered with 5 μg of plasmids encoding the WT murine FGF21 coding sequence under the control of the elongation factor 1a (EF1a) promoter (EF1a-mFGF21) (Zhang et al., EBioMedicine 15 (2017) 173-183) or a codon-optimized murine FGF21 coding sequence under the control of the CMV promoter (CMV-moFGF21) or the hAAT promoter (hAAT-moFGF21). Analysis of FGF21 expression levels in the liver 24 h post-administration of plasmids revealed that animals treated with hAAT-moFGF21 or CMV-moFGF21 expressed much higher levels of FGF21 than animals receiving EF1a-mFGF21 (FIG. 35A). In addition, animals treated with hAAT-moFGF21 or CMV-moFGF21 showed higher FGF21 circulating levels than animals receiving EF1a-mFGF21 (FIG. 35B).

### Example 24. In Vivo Increased FGF21 Expression in Target Tissues and FGF21 Circulating Levels by AAV8-hAAT-moFGF21, AAV8-CAG-moFGF21-doublemiRT and AAV1-CMC-moFGF21 in Comparison with AAV8-Ef1a-mFGF21

### Hepatic Expression

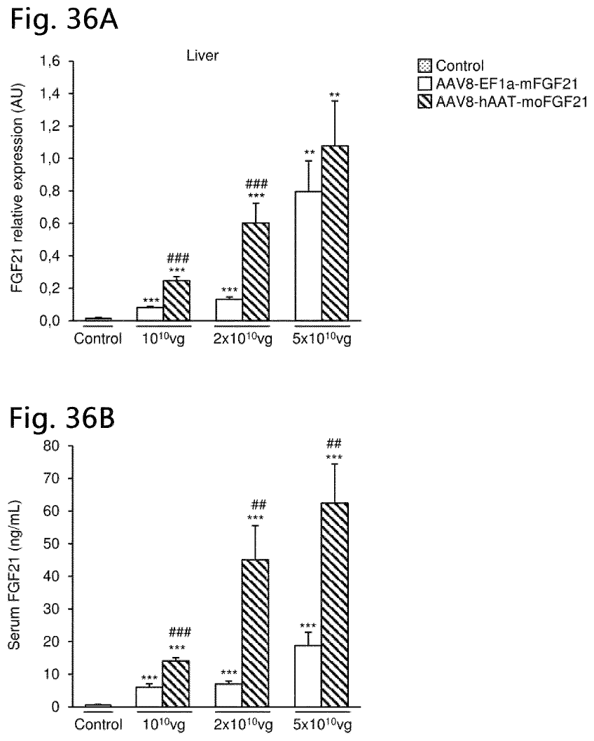

Male C57B16 mice were intravenously administered with 1×10¹⁰ vg, 2×10¹⁰ vg or 5×10¹⁰ vg of AAV8 vectors encoding the WT murine FGF21 coding sequence under the control of the elongation factor 1a (EF1a) promoter (AAV8-EF1a-mFGF21) or a codon-optimized murine FGF21 coding sequence under the control of the liver-specific hAAT promoter (AAV8-hAAT-moFGF21). Two weeks post-AAV administration, animals treated with AAV8-hAAT-moFGF21 showed both higher expression levels of FGF21 in the liver and higher FGF21 circulating levels than animals treated with AAV8-EF1a-mFGF21, irrespective of the dose of vector (FIGS. 36A and B).

### Adipose Expression

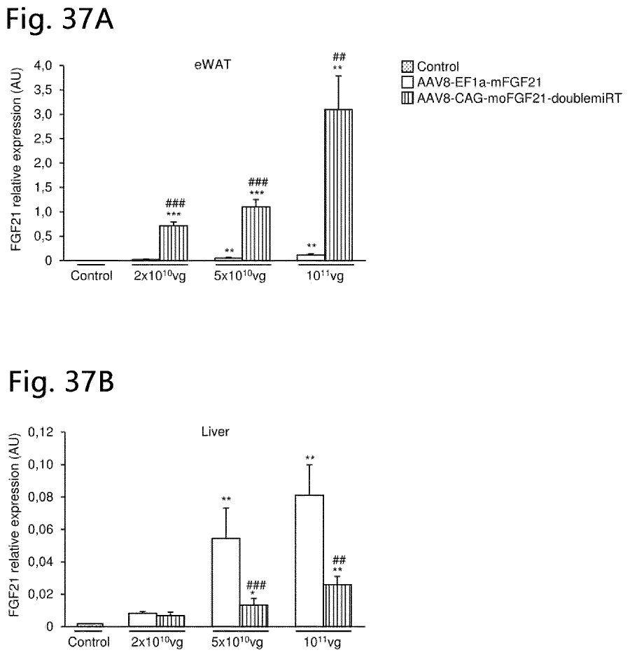

Male C57B16 mice were administered intra-eWAT with 2×10¹⁰ vg, 5×10¹⁰ vg or 1×10¹¹ vg of either AAV8 vectors encoding the WT murine FGF21 coding sequence under the control of the elongation factor 1a (EF1a) promoter (AAV8-EF1a-mFGF21) or AAV8 vectors encoding a codon-optimized murine FGF21 coding sequence under the control of the CAG promoter in conjunction with four tandem repeats of the miRT122a sequence and four tandems repeats of the miRT1 sequence (AAV8-CAG-moFGF21-doublemiRT). Two weeks post-AAV administration, animals treated with AAV8-CAG-moFGF21-doublemiRT showed higher expression levels of FGF21 in WAT than animals administered with AAV8-EF1a-mFGF21 (FIG. 37A). Moreover, animals treated with AAV8-CAG-moFGF21-doublemiRT showed much lower expression of FGF21 in the liver than animals administered with AAV8-EF1a-mFGF21 (FIG. 37B), demonstrating that intra-eWAT administration of AAV8-CAG-moFGF21-doublemiRT vectors efficiently precluded transgene expression in off-target tissues.

### Skeletal Muscle Expression

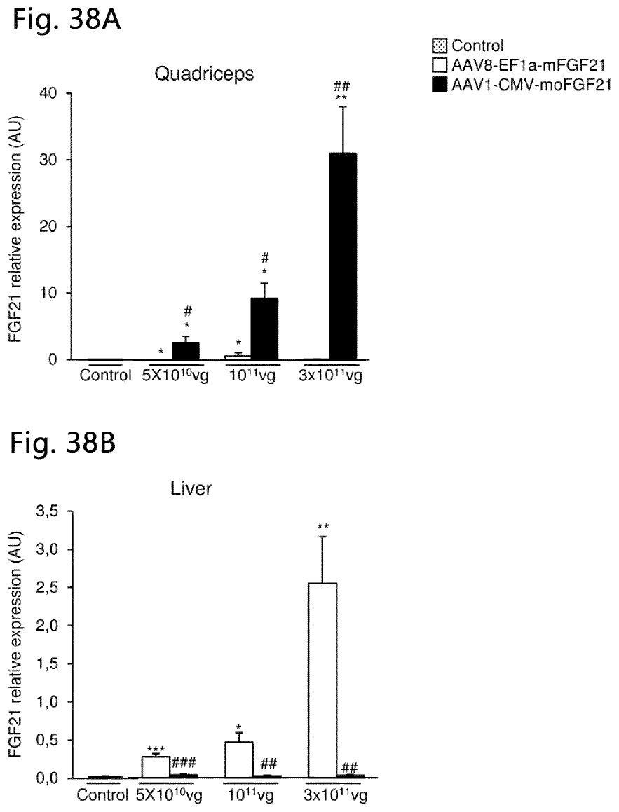

Male C57B16 mice were administered intramuscularly with 5×10¹⁰ vg, 1×10¹¹ vg or 3×10¹¹ vg of either AAV8 vectors encoding the WT murine FGF21 coding sequence under the control of the elongation factor 1a (EF1a) promoter (AAV8-EF1a-mFGF21) or AAV1 vectors encoding a codon-optimized murine FGF21 coding sequence under the control of the CMV promoter (AAV1-CMV-FGF21). Two weeks post-AAV administration, animals treated with AAV1-CMV-FGF21 showed much higher expression levels of FGF21 in skeletal muscle than animals administered with AAV8-EF1a-mFGF21 (FIG. 38A). Moreover, animals treated with AAV8-EF1a-mFGF21 showed high expression of FGF21 in the liver whereas intramuscular administration of AAV1-CMV-FGF21 vectors efficiently precluded hepatic transgene expression (FIG. 38B).

1-28. (canceled)

29. A viral expression construct comprising a nucleotide sequence encoding a Fibroblast growth factor 21 (FGF21) operably linked to at least one heterologous regulatory element selected from:
(a) a liver-specific promoter,
(b) an adipose tissue-specific promoter,
(c) a combination of a ubiquitous promoter and at least one nucleotide sequence encoding a target sequence of a microRNA expressed in the liver and at least one nucleotide sequence encoding a target sequence of a microRNA expressed in the heart,
(d) a skeletal muscle promoter, and
(e) a ubiquitous promoter.

30. The viral expression construct of claim 29, wherein the nucleotide sequence encoding the FGF21 encodes a signal sequence.

31. The viral expression construct of claim 30, wherein the nucleotide sequence encoding the FGF21 is selected from a sequence having at least 95% sequence identity with SEQ ID NO: 5, 6, or 7.

32. The viral expression construct of claim 31, wherein the nucleotide sequence encoding the FGF21 encodes SEQ ID NO: 1.

33. A recombinant adeno-associated virus (rAAV) vector comprising a vector genome and an AAV capsid, the vector genome comprising an inverted terminal repeat (ITR) and the viral expression construct of claim 29.

34. The rAAV vector of claim 33, wherein the AAV capsid has a serotype selected from AAV1, AAV2, AAV3, AAV4, AAV5, AAV8, or AAV9.

35. A nucleic acid molecule comprising a nucleotide sequence encoding a FGF21, wherein the nucleic acid molecule comprises a sequence having at least 95% sequence identity with SEQ ID NO: 5, 6, or 7.

36. The nucleic acid molecule of claim 35, comprising a sequence selected from SEQ ID NO: 5, 6, or 7.

37. The nucleic acid molecule of claim 36, further comprising one or more of a promoter, a polyadenylation signal, and/or an enhancer sequence.

38. An expression cassette comprising the nucleic acid molecule of claim 37, comprising the promoter operably linked to the nucleotide sequence encoding FGF21.

39. A recombinant adeno-associated virus (rAAV) vector comprising a vector genome encapsidated in an AAV capsid, wherein the vector genome comprises the expression cassette of claim 38 flanked by a 5′ ITR and a 3′ ITR.

40. A composition comprising the rAAV vector of claim 39 and a pharmaceutically acceptable excipient or vehicle.

41. A host cell comprising the nucleic acid molecule of claim 35.

42. A recombinant adeno-associated virus (rAAV) vector comprising a vector genome and an AAV1 serotype capsid, the vector genome comprising an inverted terminal repeat (ITR) and a viral expression construct comprising a nucleotide sequence operably linked to a ubiquitous promoter, wherein the nucleotide sequence encodes a polypeptide comprising a full-length Fibroblast growth factor 21 (FGF21) protein and a signal sequence.

43. The rAAV vector of claim 42, wherein the ubiquitous promoter comprises a cytomegalovirus (CMV) promoter or a CAG promoter.

44. The rAAV vector of claim 42, wherein the nucleotide sequence is selected from a sequence having at least 95% sequence identity with SEQ ID NO: 5, 6, or 7.

45. The rAAV vector of claim 42, wherein the nucleotide sequence is selected from SEQ ID NO: 5, 6, or 7.

46. The rAAV vector of claim 42, wherein the nucleotide sequence comprises SEQ ID NO: 5.

47. The rAAV vector of claim 42, wherein the nucleotide sequence comprises SEQ ID NO: 6.

48. The rAAV vector of claim 42, wherein the nucleotide sequence comprises SEQ ID NO: 7.

49. The rAAV vector of claim 42, wherein the nucleotide sequence encodes SEQ ID NO: 1.

50. The rAAV vector of claim 42, wherein the viral expression construct further comprises a polyadenylation signal and/or an enhancer sequence.

51. The rAAV vector of claim 42, wherein the viral expression construct is flanked by a 5′ ITR and a 3′ ITR.

52. A recombinant adeno-associated virus (rAAV) vector comprising a vector genome and an AAV1 serotype capsid, the vector genome comprising a viral expression construct flanked by a 5′ ITR and a 3′ ITR, wherein the viral expression construct comprises a nucleotide sequence operably linked to a cytomegalovirus (CMV) promoter or CAG promoter, wherein the nucleotide sequence comprises a sequence having at least 95% sequence identity with SEQ ID NO: 7.

53. The rAAV vector of claim 52, wherein the promoter is a CMV promoter.

54. The rAAV vector of claim 52, wherein the promoter is a CAG promoter.

55. The rAAV vector of claim 52, wherein the nucleotide sequence comprises SEQ ID NO: 7.

56. The rAAV vector of claim 52, wherein the nucleotide sequence encodes SEQ ID NO: 1.

57. The rAAV vector of claim 52, wherein the viral expression construct further comprises a polyadenylation signal.

58. The rAAV vector of claim 55, wherein the polyadenylation signal is a SV40 polyadenylation signal.
